(19) United States
(12) Patent Application Publication
(10) Pub. No.: US 2006/0263805 A1
(43) Pub. Date: Nov. 23, 2006

(54) Diagnosing and treating potassium channel defects

(76) Inventors: Andre Terzic, Rochester, MN (US); Timothy M. Olson, Rochester, MN (US); Garvan C. Kane, Rochester, MN (US)

Correspondence Address: FISH & RICHARDSON P.C., PO BOX 1022, MINNEAPOLIS, MN, 55440-1022, US

(21) Appl. No.: 11/377,889

(22) Filed: Mar. 16, 2006

Related U.S. Application Data
(60) Provisional application No. 60/662,279, filed on Mar. 16, 2005.

(51) Int. Cl. C12Q 1/68

(52) U.S. Cl. 435/6

(57) ABSTRACT

This documents relates to potassium channels, SUR2A polypeptides, mutant SUR2A polypeptides, and isolated nucleic acids encoding such polypeptides. In addition, this document provides methods for identifying an individual at risk for cardiac disease, methods for identifying an individual who will not respond to, or who is not responding to, conventional treatment of sepsis and/or hypertension, and methods for screening an individual that has had a poor outcome with catecholamine induction for a deficiency in cardiac K_{ATP} channeling. This document also provides methods for treating an individual for a deficiency in cardiac K_{ATP} channeling.

### CROSS REFERENCE TO RELATED APPLICATIONS

This application claims priority under 35 U.S.C. § 119(e) of U.S. provisional application Ser. No. 60/662,279, filed Mar. 16, 2005.

### TECHNICAL FIELD

This document relates to potassium channels, and more particularly to mutations that cause defects in potassium channels and methods of treating conditions related to such defects.

### BACKGROUND

Ion-channels are polypeptides embedded in cell membranes that act as extremely selective gateways, allowing specific ions to pass in and out of the cell in response to various signals. Most dramatically, they mediate the electrical impulses known as “action potentials” that are the basis of communication in the nervous system. ATP-sensitive potassium (K_{ATP}) channels have been found in a wide variety of tissues, including skeletal and smooth muscle cells, secretary cells such as insulin-secreting pancreatic β-cells, cardiac myocytes, and neurons.

Potassium currents in the brain responsible for perception and movement and potassium currents in the heart responsible for contraction rely upon the steady ebb-and-flow of potassium. Intracellular potassium ion concentration is higher (150 mM) than the extracellular potassium ion concentration (3-4 mM). Potassium channel opening results in the outflow of potassium ions and hyperpolarization, which leads to cellular responses like smooth muscle relaxation. Potassium ion channel performs the two main functions: gating-a process by which pores open and close; and permeation-selective passage of potassium ion through the pore.

Cardio-protective K_{ATP} channels in heart muscle are comprised of an SUR2A regulatory subunit encoded by ABCC9, and a pore-forming Kir6.2 subunit encoded by KCNJ11. Genetic disruption of the channel complex confers susceptibility to heart failure as demonstrated in individual patients with familial dilated cardiomyopathy and animal models of K_{ATP} channel deficiency.

### SUMMARY

This description relates to diagnosing and treating conditions related to potassium channels. For example, this description provides mutant SUR2A polypeptides as well as polymorphic Kir6.2 polypeptides. This description also provides isolated nucleic acids encoding mutant SUR2A polypeptides and polymorphic Kir6.2 polypeptides. In addition, this description provides methods for identifying an individual at risk for cardiac disease, methods of identifying an individual who will not respond to, or who is not responding to, conventional treatment of sepsis and/or hypertension, and methods of screening an individual who has had a poor outcome with catecholamine induction for a deficiency in cardiac potassium channeling. This description further provides methods for treating an individual having a deficiency in cardiac potassium channeling.

In one aspect, the invention provides methods of identifying an individual (e.g., a human) at risk for cardiac disease. Such a method typically includes determining whether or not the individual contains a mutation present in a nucleic acid encoding a SUR2A polypeptide or regulating the expression of the SUR2A polypeptide or a polymorphism in a nucleic acid encoding a Kir6.2 polypeptide. Generally, the presence of the mutation indicates that the individual is at risk for cardiac disease. Representative cardiac diseases include, without limitation, heart failure, ventricular arrhythmias, or atrial arrhythmias (e.g., atrial fibrillation).

In another aspect, the invention provides methods for identifying an individual who will not respond to, or who is not responding to conventional treatment of sepsis or hypertension. Such a method generally includes determining whether or not the individual contains a mutation present in a nucleic acid encoding a SUR2A polypeptide or regulating the expression of the SUR2A polypeptide or a polymorphism in a nucleic acid encoding a Kir6.2 polypeptide. Typically, the presence of the mutation indicates that the individual is identified as one who will not respond to or who is not responding to conventional treatment of sepsis or hypertension. Such a method can further include modifying the treatment of the individual for sepsis or hypertension based on the identification.

In another aspect, the invention provides for methods for screening an individual for a deficiency in cardiac K_{ATP} channeling, wherein the individual has experienced a poor outcome with catecholamine induction. Such a method typically includes determining whether or not the individual contains a mutation present in a nucleic acid encoding a SUR2A polypeptide or regulating the expression of a SUR2A polypeptide or a polymorphism in a nucleic acid encoding a Kir6.2 polypeptide. Generally, the presence of the mutation indicates that the individual is identified as having a deficiency in cardiac K_{ATP} channeling.

In yet another aspect, the invention provides for methods of treating an individual having a deficiency in cardiac K_{ATP} channeling. Such a method generally includes identifying an individual as having the deficiency, and administering an effective dose of a potassium channel opener (PCO) to the individual. Typically, the PCO improves cardiac K_{ATP} channeling, thereby treating the individual for the deficiency in cardiac K_{ATP} channeling. Such a method can further include monitoring the individual for cardiac K_{ATP} channeling; and administering a calcium channel blocker if the individual does not respond to the PCO.

Representative mutations present in the nucleic acid can encode a SUR2A polypeptide resulting in Fs1524, Ala1513Thr, or Thr1547Ile. Representative polymorphisms present in the nucleic acid can encode a Kir6.2 polypeptide resulting in E23K. Representative methods of detecting mutations include, without limitation, sequencing, electrophoretic mobility, nucleic acid hybridization, fluorescent in situ hybridization, polymerase chain reaction, reverse transcription-polymerase chain reaction, denaturing high-performance liquid chromatography, or a combination thereof.

In another aspect, the invention provides an isolated nucleic acid comprising a sequence encoding a mammalian SUR2A polypeptide, wherein the sequence comprises a mutation that disrupts the function of an assembled K_{ATP} channel. The invention also provides an islated nucleic acid comprising a sequence encoding a mammalian Kir6.2 polypeptide, wherein the sequence comprises a polymorphism that disrupts the function of an assembled K_{ATP} channel.

For example, a mutation in a nucleic acid of the invention can result in a frameshift at the codon encoding Leucine 1524 (Fs1524) in the reference sequence set forth in SEQ ID NO:11. Such a frameshift can be due to a 3 basepair deletion and a 4 basepair insertion at position 4570-4572 (4570-4572delTTAinsAAAT) in the reference sequence set forth in SEQ ID NO: 10. See, for example, the nucleotide sequence set forth in SEQ ID NO:12.

For example, a mutation in a nucleic acid of the invention can result in an amino acid substitution of Thr to Ala at position 1513 (Ala1513Thr) in the reference sequence set forth in SEQ ID NO:11. Such a substitution can be caused by a G to A missense mutation at position 4537 in the reference sequence set forth in SEQ ID NO:12. See, for example, the nucleotide sequence set forth in SEQ ID NO:14.

In yet another aspect, the invention provides for a substantially pure mammalian SUR2A polypeptide, wherein the polypeptide comprises a mutation that disrupts the function of an assembled K_{ATP} channel. For example, such a mutation can reflect a frameshift in the nucleic acid encoding the SUR2A polypeptide at the codon encoding Leu1524. See, for example, the amino acid sequence set forth in SEQ ID NO:13. For example, such a mutation can be an Ala1513Thr substitution. See, for example, the amino acid sequence set forth in SEQ ID NO:15. In another aspect, the invention provides for a substantially pure mammalian Kir6.2 polypeptide, wherein the polypeptide comprises a mutation that disrupts the function of an assembled K_{ATP} channel.

The invention also provides for a nucleic acid construct comprising a nucleic acid of the invention, and a host cell comprising such a nucleic acid construct. The invention further provides for an antibody that binds to a mutant SUR2A polypeptide but does not bind to a polypeptide having an aimino acid sequence as set forth in SEQ ID NO:11. The invention further provides for an antibody that binds to a polymorphic Kir6.2 polypeptide but does not bind to a wild type Kir6.2 polypeptide. Such an antibody can be, without limitation, a monoclonal antibody, a humanized antibody, a chimeric antibody, an antibody fragment, a single chain antibody, or a single domain antibody. Antibody fragments can include a Fab fragment, a (Fab′)₂ fragment, or a Fv fragment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 shows various features of the K_{ATP} channel mutations identified herein. (A) The regulatory SUR2A subunit (nucleotide-binding domains: NBD1/NBD2 with Walker A/B motifs and a linker L region) forms cardiac K_{ATP} channels by assembling with the pore-forming Kir6.2 subunit (transmembrane domains: M1/M2). Genomic DNA analysis of exon 38 in ABCC9, which encodes the carboxy-terminus of the SUR2A polypeptide, identified abnormal chromatograms indicative of mutations in patients with dilated cardiomyopathy (DCM). Sequencing demonstrated frameshift (Fs1524; Patient #1) and missense (A1513T; Patient #2) mutations. The family of Patient #1 was unavailable for segregation analysis. The mutation in Patient #2 was not present in the proband's mother suggesting inheritance from the affected father (DNA unavailable). (B) SUR2A residues encoded by exon 38 in human wild-type (SEQ ID NO:3) and mutants ABCC9 (SEQ ID NOs:4 and 5) versus other species (SEQ ID NOs:6-9).

FIG. 2 shows the biological effects of the SUR2A mutations. (A) Fs1524 and A1513T reduced by ˜70% and ˜30% K_{ATP} channel trafficking probed immunologically by SUR Xenopus oocyte surface expression. (B) Single channel conductance and inward-rectification (not shown) of wild-type and mutated channels, expressed in HEK293 cells, were identical indicating intact biophysical pore properties. (C-D) Atomic model of SUR2A NBD2. COOH: carboxy-terminus, red: α-helix, blue: β-strand, yellow W_{A}/W_{B}: Walker motifs. Missense A1513T (cyan) and frameshift L1524 (magenta) mutations frame the β-strand adjacent to Walker motifs that coordinate NBD2-mediated catalysis. Representative hydrogen bonds that stabilize W_{A} and the associated carboxy-terminus α-strand are indicated by dashed green lines. Oxygen atoms: red, nitrogen: blue and sulfur (cysteine 1345): mustard. (E) Abnormal ATP-induced K_{ATP} channel inhibition in mutants. Channel inhibition was expressed relative to activity in the absence of ATP, and measured at −60 mV in inside-out patches. Solid curves represent Hill equation fits of experimental data, with the ATP concentration required for half-inhibition (IC₅₀) indicated for wild-type and mutant channels.

FIG. 3 presents data demonstrating that SUR2A NBD2 mutants exhibit normal ATP-binding, but altered ATPase properties. (A) Affinity purified mutant or wild-type SUR2A NBD2, cloned in-frame with the maltose binding protein (MBP), on SDS gels. An antibody against the last 12 amino acids of SUR2A recognized the wild-type and A1513T mutant but not Fs1524, whereas an antibody raised against MBP reacted with all constructs. (B) The binding affinity of the fluorescent ATP analog, TNP-ATP, was similar for wild-type and mutated NBD2 in Scatchard analysis. Inset: Specific TNP-ATP binding-induced signal detected as difference between total and non-specific (NS) fluorescence of the ATP analog in the absence of NBD constructs. (C) A1513T and Fs1524 NBD2 mutations reduced ATPase activity measured from γ-³²P liberation following [γ-³²P]ATP hydrolysis. (D-E) ATP and ADP dependence of NBD2 ATPase activities measured by spectrophotometry revealed a Vₘₐₓ of 9.98±0.34 min⁻¹ in wild-type, 6.07±0.18 min⁻¹ in A1513T and 5.69±0.29 min⁻¹ in Fs1524 with Michaelis-Menten constants at 0.11±0.02, 0.094±0.013 and 0.084±0.010 mM, respectively (Equation 3 in Methods). The ATP-dependence of the NBD2 ATPase was determined at 0 ADP in the presence of creatine kinase (0.01 U/ml) and creatine phosphate (5 mM). ADP-dependent inhibition of the NBD2 ATPase (at 2 mM ATP) was characterized by an ADP-dissociation constant (K_{ADP}) of 8.6±0.3 μM in wild-type versus 24.9±5.6 and 11.0±1.5 in A1513T and Fs1524 mutants, respectively (Equation 5). (F) Both pre- and steady-state reaction rates were altered by A1513T and Fs1524 mutations in stopped-flow experiments. Solid lines represent the fit of experimental data with the system of differential equations (Equation 6) allowing evaluation of the rate kinetic constants of the NBD2 ATPase reaction.

FIG. 4 demonstrates that altered kinetics of the ATPase cycle lead to disrupted metabolic decoding by K_{ATP} channels. (A) Rate constants defining the SUR2A ATPase reaction were derived from pre-steady state kinetics of Pᵢ liberation in wild-type and mutated NBD2 constructs (n=4 each). (B) Lifetime distribution of individual conformations (Equation 10) in wild-type and mutated NBD2 ATPase cycle. (C) Rate limiting steps in the SUR2A ATPase cycle are: ADP dissociation (k₄) for wild-type, Pᵢ dissociation (k₃) for Fs1524, and abnormally increased k₄ for A1513T. In addition, Fs1524 displays the lowest k₀₄ rate constant defining ADP association. (D) In the absence of ADP, both NBD2 mutants exhibit lower probability to adopt ATP-(P_{E·ATP}) and higher probability to adopt ADP-bound (P_{ΣADP}) conformations in a wide range of ATP concentrations. P_{ΣADP} is the sum of E·ADP·Pᵢ (P_{E·ADP·Pi}) and E·ADP (P_{E·ADP}) probabilities (Equations 7-9). (E-F) ADP-induced modulation of probabilities in the NBD2 ATPase cycle intermediates determined as dP_{ΣADP}/d[ADP] and dP_{E·ATP}/d[ADP] derivatives at different ATP levels. In response to ADP, P_{ΣADP} increases whereas P_{E·ATP} decreases defining the sign of respective derivatives. Both Fs1524 and A1513T diminished the ADP-responsiveness of _{ΣADP} and P_{E·ATP}. (G) ADP-scavenging creatine kinase (0.01 U/ml, 5 mM creatine phosphate) accelerates the ATPase in wild-type, but not in Fs1524 and A1513T mutants. Rate of Pᵢ liberation was measured at 2 mM ATP using spectrophotometry. (H-I) Channel activity, at 0.3 mM ATP, in the presence and absence of 0.3 mM ADP measured in wild-type (n=5), Fs1524 (n=4) and A1513T (n=6) SUR2A mutants in inside-out patches. In addition to reduced ATP sensitivity, both mutants exhibited blunted ADP channel response shown relative to K_{ATP} channel activity at zero nucleotide levels.

FIG. 5 shows graphs that demonstrate the survival disadvantage with compromised cardiac activity in endotoxic Kir6.1-KO. The Escherichia coli LPS endotoxin produces premature and pronounced mortality in Kir6.1-KO compared to time-matched wild-type (WT) mice (A; P<0.0001). Compared to WT, the Kir6.1-KO displays a high susceptibility to a dose range of LPS (B; P<0.01) with a readily demonstrable early death (C; P<0.006). Preceding LPS induced death, cardiac activity progressively declines in the Kir6.1-KO, not observed in WT, as captured by telemetry for individual cases (D) or collectively in respective cohorts (E). WT manifests normal P, QRS and T wave patterns on continuous lead II and III electrocardiograms at baseline and following LPS (F upper row). Kir6.1-KO, which in the majority of time monitored show unremarkable electrocardiograms at baseline (characteristic but short-lived and episodic ST changes are not shown), demonstrate under LPS challenge marked and persistent ST segment elevation (F lower row), highlighted in tracings magnified in insets.

FIG. 6 demonstrates that ischemic cardiac dysfunction precedes LPS-induced death in Kir6.1-KO. Myocyte necrosis on light (20×; arrows in A) and electron (B) microscopy with mitochondrial swelling (B, inset) in Kir6.1-KO (KO), but not wild-type (WT) myocardial samples at 4-6 h following LPS. In B, inset bar corresponds to 1 μm. Unlike WT, endotoxic-KO mice fail to initially augment cardiac performance, and progress to severe depression of ventricular function measured by left ventricular fractional shortening (LVFS, P<0.05; C and D) and circumferential shortening velocity (Vcf, P<0.05; C and E). In C, full and open arrows indicate diastolic and systolic dimensions, respectively. In D, * and ** indicate significant difference from time 0 and corresponding WT, respectively. Grey shading depicts range of normal contractile function.

FIG. 7 shows graphs demonstrating the loss of cytokine-induced vasodilation in Kir6.1-KO. Comparable levels of the cytokine tumor necrosis factor alpha (TNFα) in sera of wild-type (WT) and Kir6.1-KO mice prior to and following induction of the endotoxic state (A). For each time point, 3-5 WT or KO mice are sampled. WT hearts respond to TNFα, with maximal coronary vasodilation (B and C). No increase in coronary flow under TNFα observed in Kir6.1-KO hearts or WT hearts pre-treated with the K_{ATP} channel inhibitor, glyburide (Gly; B and C). LPS-challenged Kir6.1-KO mice demonstrate no systemic vascular response measured by systolic blood pressure (D).

FIG. 8 shows graphs demonstrating that the Kir6.1-KO phenotype was rescued by restoration of the dilatory capacity of coronary vessels. The calcium channel antagonist, verapamil, restores coronary vasodilation in tumor necrosis factor alpha (TNFα)-challenged Kir6.1-KO (A) negating premature LPS-induced mortality (B). Rescue experiments are based on a total of 14 mice.

FIG. 9 shows the survival disadvantage of Kir6.2-KO mice following mineralocorticoid salt loading. Mineralocorticoid/salt (DOCA/salt) challenge induces significant but comparable systemic hypertension (A), fluid intake (B), renal hypertrophy (C) with kidney collagen deposition (D) in wild-type (WT) and K_{ATP} channel knockout (Kir6.2-KO) mice. In response to hypertension, Kir6.2-KO (KO) mice display a compromised exercise capacity on treadmill testing (E) and severe mortality (F; P<0.001) compared to WT. Star indicates significant difference compared to control (P<0.001). Treadmill exercise capacity expressed as change from mean of unchallenged WT or KO controls.

FIG. 10 shows that hypertension precipitates congestive heart failure in the absence of K_{ATP} channels. Preceding death, hypertensive K_{ATP} channel knockout (KO) mice demonstrate progressive bradycardia (A) with atrio-ventricular conduction delay (A inset), not observed in wild-type (WT). In hypertensive KO (KO HTN) but not WT (WT HTN), echocardiography-captured left ventricular (LV) dilatation (B; open arrows: LV chamber dimension in diastole and closed arrow: LV chamber dimension in systole), impaired trans-aortic valve velocity (C), and fractional shortening (D), with congested lungs (E). On cardiac catheterization, impaired inotropy (F, diminished LV developed LV_{dev} pressure), and lusitropy (G, prolonged cardiac relaxation) in hypertensive KO (H). With dobutamine challenge, KO rapidly decompensates into overt cardiac failure (I). Star indicates significant difference compared to control (P<0.01). Horizontal scale bars in A inset, B, C, H and I correspond to 200 ms. Vertical scale bars in B correspond to 1 mm and in C to 2 mm/s.

FIG. 11 shows the exaggerated maladaptive left ventricular remodeling in hypertensive K_{ATP} channel knockout. K_{ATP} channel knockout (KO) challenged with hypertension (HTN), develop aggravated cardiac (A), and left ventricular (LV; B) hypertrophy. Hypertension-stressed KO, immunostaining negative for Kir6.2, have larger cardiomyocyte surface area compared to Kir6.2-positive wild-type (WT; C). Scale bar in C corresponds to 20 μm. Masson's trichrome stained hypertensive KO hearts are significantly more fibrotic than WT (D and E). Star indicates significant difference compared to respective control or hypertensive WT (P<0.05).

FIG. 12 shows that the absence of metabolic distress-induced K_{ATP} channel-dependent repolarization precipitates excessive calcium loading and triggers activation of remodeling signals. Altered bioenergetics probed by the nucleotide diphosphate (DP) to triphosphate (TP) index in hypertensive wild-type (WT) and Kir6.2-knockout (KO) hearts versus respective controls (A; P<0.005). Prolongation of corrected QT interval (QTc) in hypertensive KO compared to WT (B; P=0.003) with calcium accumulation in hypertensive KO versus WT cardiomyocytes captured by the fluorescent probe Fluo-4 on laser confocal microscopy (C; P<0.001). Nuclear translocation of the calcium-dependent cardiac transcription factor (MEF2), highlighted in insets, in α-actinin stained left ventricular tissue sections (D) greater in hypertensive KO versus WT (P<0.001).

FIG. 13 shows that exaggerated activation of calcium-calcineurin dependent hypertrophic signaling in hypertensive hearts lacking K_{ATP} channels. Calcineurin phosphatase activity significantly increased in Kir6.2 knockout (KO) versus wild-type (WT) hearts following hypertension (A), with a tight correlation between calcineurin enzyme activity and resulting hypertrophy (B). Change in calcineurin activity expressed as % change from mean of unchallenged WT or KO control groups. Enhanced cytosol to nucleus shuttling of NF-AT in KO confirmed by co-localization with the nuclear marker DAPI, in α-actinin stained isolated cardiomyocytes (C), highlighted in insets. Scale bars in C indicate 20 μm. Immunoblot analysis of nuclear extracts confirms induction of nuclear localization of NFAT in hypertensive KO (C). WT treated with the same hypertrophic protocol show only modest nuclear accumulation of NFAT3 as compared to untreated, control mice (representative gel, D; quantitation, E). Protein molecular weight standard is indicated to the right in kD. Electrophoretic mobility shift assay in conjuction with immunodepletion (F) demonstrate NF-AT3 binding the NF-AT consensus sequence in promoter region of the b-type natriuretic protein gene (BNP). Specificity is determined by preincubation with a 50-fold molar excess of either the unlabeled wild-type sequence (W) or a mutated consensus NF-AT sequence (M). The dash (−) indicates that no competitor or antibody was added. Depletion (D) of the nuclear extract of NF-AT3 by immunoprecipitation prevents DNA (probe) binding. DNA binding is recovered (R) by adding the immunoprecipitate (ie., enriched in NF-AT3) to the probe. The DNA/protein complex (bracket) and unbound DNA probe (arrow) are indicated to the right.

FIG. 14 shows that calcineurin inhibition negates excessive remodeling in K_{ATP} channel knockout. In the hypertensive (HTN) K_{ATP} channel knockout (KO), NF-AT nuclear accumulation (boxed in A) prevented by calcineurin inhibition with daily cyclosporine A (CsA) therapy (B). In the absence of CsA treatment (HTN-CsA) KO demonstrate excessive cardiac (C) and left ventricular (LV, D) hypertrophy compared to wild-type (WT), with differences negated by daily CsA treatment (HTN+CsA). Change in cardiac or LV mass expressed as % change from mean of unchallenged WT or KO controls. Star indicates significant difference compared to respective hypertensive WT (P<0.005).

FIG. 15 shows that calcium channel antagonism rescues the hypertensive K_{ATP}channel-knockout phenotype. Scheme (A) depicts proposed role for K_{ATP} channel-dependent regulation of calcium homeostasis under metabolic stress. Sensing changes in the cellular bioenergetic state (e.g., through relative increases in nucleotide diphosphates, such as ADP, at the channel site) K_{ATP} channel activity promotes shortening of the cardiac action potential duration (APD) corresponding to shortening of the QT interval on electrocardiograms and a net decrease in calcium entry through voltage-dependent calcium channels. Treatment with the calcium channel antagonist verapamil (ver) prevents aggravated hypertrophy (B inset), pulmonary congestion (B inset) and death (B; P=0.03) in hypertensive Kir6.2 knockout (KO). Changes in left ventricular (LV) mass and lung weight (wgt) expressed as % change from mean of unchallenged KO controls. Star indicates significant difference compared to respective control or hypertensive WT (P<0.001).

FIG. 16 shows that the exercise capacity in aquatic endurance-trained Kir6.2-KO mice is impaired. In wild-type (WT) and Kir6.2-KO (KO) mice, a 28-day swimming protocol (A) produced enhanced skeletal muscle metabolic capacity as measured by succinate dehydrogenase (SDH) activity (B, *P<0.05), a reduction in basal heart rate (C, *P<0.01) and better performance on treadmill stress testing (D, P<0.05 from control; * P<0.05 versus WT swim). Yet, Kir6.2-KO improved significantly less than WT (D; P<0.05).

FIG. 17 shows that training-induced improvements in body weight and fat distribution were absent in Kir6.2-KO. In contrast to wild-type (WT), the Kir6.2-KO (KO) mice failed to lose body weight with swimming training (A, *P<0.001), and displayed less leptin/body weight correlation (B). Only WT demonstrated measurable reductions in epididymal white fat deposits (C) and a decline in white adipocyte size (C and D; P<0.01). SED, sedentary; SW, swimming.

FIG. 18 shows that Kir6.2-KO mice lack the metabolic benefits of physical activity and develop skeletal muscle damage. The swimming protocol elicits a significant reduction in fasting blood glucose in wild-type (WT; P<0.05), an effect not experienced in the Kir6.2-KO (KO; A). All WT have a normal response to a glucose tolerance test (GTT), while the glucose intolerance of Kir6.2-KO is not improved by exercise training (B). SED, sedentary; SW, swimming. Training exaggerated the abnormal drop in glucose following an insulin tolerance test (ITT) in Kir6.2-KO, without affecting WT (C; *P<0.05 from baseline, **P<0.05 between Kir6.2-KO groups). Participation in swimming resulted in myocellular damage to the Kir6.2-KO (KO SW), with focal areas of myocyte degeneration and vacuolar destruction not observed in the sedentary (KO SED) on histology (arrows in D, E and F). Kir6.2-KO had cells with basophilic cytoplasm, large nuclei and prominent nucleoli (D and E), findings typical of early regenerative changes confirmed by abnormal central internalized nuclei on electron microscopy (G lower) compared to the normal peripheral location in the KO SED (G upper). In G, arrows indicated nuclei.

FIG. 19 shows the cardiac dysfunction and survival disadvantage of exercised Kir6.2-KO mice. After 28 days of the swimming protocol, Kir6.2-KO had impairment in cardiac function with reduced left ventricular shortening fraction (A; P<0.02), and cardiac output (B; P<0.01) compared to wild-type or unstressed Kir6.2-KO. Indeed, swimming induced a significant survival disadvantage in Kir6.2-KO over sedentary controls (C; P<0.02).

FIG. 20 shows the structural cardiac deficits in Kir6.2-KO mice with hypertrophy and focal contraction band necrosis following swimming. Unlike wild-type (WT), Kir6.2-KO (KO) hearts displayed enlarged cardiac and left ventricular (LV) mass to swimming stress (A), and focal areas of contraction band necrosis seen on light (arrows in B left) and electron microscopy (box in B right). SED, sedentary; SW, swimming. There was an increased expression of the transcription regulator of pathologic cardiac hypertrophy, myocyte enhancing factor (MEF2C), in left ventricular tissue extracts (C), with evidence of nuclear localization of MEF2C on immunostaining confirmed by co-localization with the nuclear marker DAPI in α-actinin stained tissue sections (D).

FIG. 21 shows that the isoproterenol challenge induced action potential shortening (APD90) in wild-type (WT; A), but not KATP channel-knockout (Kir6.2-KO; B) hearts, which developed early afterdepolarizations (EAD; C). D, Incidence of EAD in the initial fifty action potentials following 5 min of isoproterenol infusion.

FIG. 22 shows similar coronary flow in the absence and presence of isoproterenol (1 mM) in WT and Kir6.2-KO hearts (A). Isoproterenol-induced abnormal repolarization with early afterdepolarization in an isolated and current-clamped Kir6.2-KO cardiomyocyte (B).

FIG. 23 shows that Kir6.2-KO hearts with prominent EAD demonstrated triggered activity on monophasic action potentials (MAP) and premature ventricular complexes (PVC) on electrograms (EG). For comparison, an EG is shown from WT hearts that are not prone to EAD/triggered activity/PVC (A). Incidence of triggered activity with accompanying PVC in the initial fifty action potentials following 5 min of isoproterenol infusion (B).

FIG. 24 shows atrial fibrillation originating from vein of Marshall in a patient harboring a mutation in ABBC9 encoding the K_{ATP} channel subunit SUR2A. Atrial fibrillation with variable ventricular rate response was provoked by infusion of isoproterenol (Panel A). Intracardiac mapping (Panel B) localized the site of earliest activation of premature beats, initiating atrial fibrillation, to the vein of Marshall (arrow, Panel C). In B, SVC and IVC denote superior and inferior vena cava, LA left atrium, CSO coronary sinus ostium, LV left ventricle, RVA right ventricular apex catheter, HRA high right atrial catheter, HIS His bundle recording catheter, Map mapping catheter, and CS coronary sinus catheter. In C, V1 surface electrocardiogram, RVA right ventricular apex catheter, HRA high right atrial catheter, HIS His bundle recording catheter, Map p and Map d proximal and distal mapping catheter, and CS coronary sinus catheter. Sequencing of patient's DNA identified a heterozygous missense mutation (4640C>T) causing substitution of the 1547 threonine residue with isoleucine (T1547I) in exon 38 of ABCC9, encoding the carboxy-terminus of the K_{ATP} channel subunit SUR2A (Panel D). Alignment of the human carboxy-terminal domain of the SUR2A protein with orthologs from other species demonstrates conservation of the substituted threonine 1547 residue and the surrounding region (Panel E). Atomic model of the SUR2A nucleotide-binding domain 2 (NBD2) with the carboxy-terminal tail (Panel F). Red denotes α-helix, blue-green β-strand, and yellow W_{A} or W_{B} Walker motifs (left, Panel F). The threonine T1547 residue, found substituted in the patient, maps to the tail region of the SUR2A molecule adjacent to Walker motifs. Representative hydrogen bonds that stabilize Walker A and the associated carboxy-terminus are indicated by dashed lines. Red denotes oxygen atoms, royal blue nitrogen, and mustard sulfur (right, Panel F). Alignment of human SUR isoforms indicates that the carboxy-terminal tail, including threonine T1547 (arrow), is unique to the cardiac SUR2A subunit (Panel G).

FIG. 25 shows that ABCC9 mutation disrupts K_{ATP} channel function with disease phenotype verified in adrenergically-stressed gene knockout model. Panel A: cardiac K_{ATP} channels are composed of the ABCC9-encoded SUR2A subunit (containing nucleotide-binding domains: NBD1/NBD2 with Walker A/B motifs and linker L region), and the KCNJ11-encoded pore-forming Kir6.2 subunit. Under normal conditions, intracellular ATP keeps K_{ATP} channels closed. Under metabolic stress, the interaction of accumulated MgADP at NBD2 of SUR2A antagonizes ATP-induced channel inhibition, promoting K⁺ efflux. Panel B: compared to wildtype (WT), reconstituted mutant SUR2A co-expressed with Kir6.2 (T1547I) demonstrate aberrant K_{ATP} channel function, characterized by defective responsiveness to ADP in patch-clamp recordings. Panel C: reduced potassium current response to escalating ADP concentrations in mutant (T15471) versus wildtype (WT) K_{ATP} channels. Panel D: ATP-induced channel inhibition was retained in mutant channels. Channel inhibition was expressed relative to activity recorded in the absence of ATP, at −60 mV in inside-out patches. Curves represent Hill equation fits of data. Panel E: the identified T1547I mutation, while not preventing ATP-induced K_{ATP} channel closure, compromises ADP-dependent channel opening. Panel F: on telemetric monitoring, wildtype (WT) mice maintained normal sinus rhythm following isoproterenol (10 mg/kg) injection whereas Kir6.2^{-/-} knockouts lacking functional K_{ATP} channels (KO) demonstrated with sympathomimetic challenge electrical vulnerability manifested by atrial fibrillation. Panel G: K_{ATP} channel deficit led to atrial fibrillation in 70% of the knockouts (KO) within 20 min of adrenergically-medicated stress. Sinus rhythm was maintained in all wildtype mice with intact K_{ATP} channels (WT).

FIG. 26 shows that ablation at the vein of Marshall eliminated the arrhythmogenic trigger for adrenergic atrial fibrillation in the patient with mutated K_{ATP} channel. Panel A: fluoroscopic view of mapping catheter (Map) within vein of Marshall at the site of earliest atrial activation during isoproterenol-induced ectopy initiating atrial fibrillation. HRA denotes high right atrial catheter, HIS His bundle recording catheter, CS coronary sinus catheter, and RVA right ventricular apex catheter. Panel B: multi-lead intracardiac tracings before, during, and after radiofrequency ablation. Mapping: spontaneous atrial ectopy is first recorded (dotted line) in the ablation catheter (ABL), overlaying the vein of Marshall, where an abnormal potential (arrow) precedes atrial activation. Ablation: radiofrequency ablation at this site eliminated the vein of Marshall potential and atrial ectopy. Post-ablation: despite isoproterenol infusion (2 μg/min), the vein of Marshall potential or ectopy could no longer be induced and the patient remained in sinus rhythm. V1 denotes surface electrocardiogram, RVA right ventricular apex catheter, HRA high right atrial catheter, HIS His bundle recording catheter, LS left superior pulmonary vein catheter (LS1,2 . . . LS9,10—distal . . . proximal), ABL p and ABL d proximal and distal electrodes of ablation catheter, and CS coronary sinus catheter (CS 1,2 . . . CS9,10—distal . . . proximal). Panel C: adrenergic challenge provoked ectopy from the vein of Marshall, triggering transition from sinus rhythm to atrial fibrillation in this susceptible patient with inadequate responsiveness to imposed stress load. While stress-responsive K_{ATP} channels preserve electrical homeostasis, channel malfunction resulting from the ABCC9 mutation created a risk-conferring substrate predisposing to arrhythmia. Ablation of the arrhythmia-triggering focus disrupted the arrhythmogenic gene-environment interaction.

Like reference symbols in the various drawings indicate like elements.

### DETAILED DESCRIPTION

This description relates to diagnosing and treating conditions related to potassium channels. Cardio-protective K_{ATP} channels in heart muscle are comprised of an SUR2A regulatory subunit encoded by ABCC9, and a pore-forming Kir6.2 subunit encoded by KCNJ11. This description, for example, provides mutant SUR2A and polymorphic Kir6.2 polypeptides. This description also provides isolated nucleic acids encoding mutant SUR2A polypeptides and isolated nucleic acids encoding polymorphic Kir6.2 polypeptides. In addition, this description provides methods for identifying an individual at risk for cardiac disease, methods of identifying an individual who will not respond to or who is not responding to conventional treatment of sepsis and/or hypertension, and methods of screening in individual who has had a poor outcome with catecholamine induction for a deficiency in cardiac K_{ATP} channeling. This description further provides methods for treating an individual having a deficiency in cardiac K_{ATP} channeling.

Nucleic Acids and Polypeptides

This description provides isolated nucleic acid molecules that encode a SUR2A polypeptide (e.g., SEQ ID NO:10) and isolated nucleic acid molecules that encode a Kir6.2 polypeptide. A nucleic acid molecule encoding a SUR2A polypeptide can include one or more mutations such as those provided in SEQ ID NOs:12, 14, and 16. As used herein, the term “isolated” as used in reference to a nucleic acid refers to nucleic acid that is separated from other nucleic acid that is present in a mammalian genome, including nucleic acids that normally flank one or both sides of the nucleic acid in a mammalian genome (e.g., nucleic acids that encode non-SUR2A polypeptides). The term “isolated” as used herein with respect to nucleic acids also includes any non-naturally-occurring nucleic acid sequence since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, cDNA, or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

The nucleic acids provided herein can be at least about 8 nucleotides in length. For example, a nucleic acid can be about 8, 9, 10-20 (e.g., 11, 12, 13, 14, 15, 16, 17, 18, 19, or 20 nucleotides in length), 20-50, 50-100 or greater than 100 nucleotides in length (e.g., greater than 150, 200, 250, 300, 350, 400, 450, 500, 750, 1000, 2000, 3000, or 4000 nucleotides in length). In some embodiments, a nucleic acid can be in a sense or antisense orientation, can be complementary to a reference sequence encoding a SUR2A polypeptide (e.g., SEQ ID NO:10), and can be DNA, RNA, or nucleic acid analogs. Nucleic acid analogs can be modified at the base moiety, sugar moiety, or phosphate backbone to improve, for example, stability, hybridization, or solubility of the nucleic acid.

Types of mutations that a nucleic acid encoding a SUR2A polypeptide can carry include, without limitation, insertions, deletions, transitions, transversions and inversions. A nucleic acid encoding a SUR2A polypeptide can include more than one mutation and more than one type of mutation. Such mutations, if present within the coding sequence, can result in insertions or deletions of one or more amino acids of a SUR2A polypeptide, conservative or non-conservative amino acid substitutions within a SUR2A polypeptide, or premature termination of a SUR2A polypeptide. Insertion or deletion of amino acids can, for example, disrupt the conformation of essential α-helical or β-pleated sheet regions, and can also disrupt binding or catalytic sites important for enzymatic activity. Non-conservative amino acid substitutions can result in a substantial change in the bulk of the residue side chain, and ultimately can make a substantial change in the charge, hydrophobicity, or structure of a polypeptide. Premature termination also can cause disruptions in secondary and tertiary polypeptide structure. In addition, non-coding sequence mutations (e.g., mutations in a promoter, regulatory element, or untranslated region) can alter the expression pattern properties (e.g., temporal, spatial, or developmental) of a SUR2A polypeptide, by, for example, changing the binding characteristics of a cis-acting transcription factor.

In some embodiments, a nucleic acid molecule provided herein can have at least 95% (e.g., 96%, 97%, 98%, 99%, 99.5%, 99.6%, 99.7%, 99.8%, 99.9%, or 100%) sequence identity with a region of a reference sequence (e.g., SEQ ID NO:10 (GenBank Accession No. NM_{—}005691.1)), provided that the region includes one or more mutations. Such mutations are those, for example, described herein. The region is at least ten nucleotides in length (e.g., 10, 15, 20, 50, 60, 70, 75, 100, 150, 200, 250, 300, 350, 400, 450, 500, or more than 500 nucleotides in length).

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The alignment of two or more sequences to determine percent sequence identity is performed using the algorithm described by Altschul et al. (1997, Nucleic Acids Res., 25:3389-3402) as incorporated into BLAST (basic local alignment search tool) programs, available at ncbi.nlm.nih.gov on the World Wide Web. BLAST searches can be performed to determine percent sequence identity between a nucleic acid encoding a SUR2A polypeptide and any other sequence or portion thereof aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a sequence provided herein and another sequence, the default parameters of the respective programs are used.

The isolated nucleic acids provided herein can be produced by standard techniques, including, without limitation, common molecular cloning and chemical nucleic acid synthesis techniques. For example, polymerase chain reaction (PCR) techniques can be used to obtain an isolated nucleic acid containing a mutation. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize cDNA strands. Ligase chain reaction, strand displacement amplification, self-sustained sequence replication, or nucleic acid sequence-based amplification also can be used to obtain isolated nucleic acids. See, for example, Lewis, Genetic Engineering News, 12(9):1 (1992); Guatelli et al., Proc. Natl. Acad. Sci. USA, 87:1874-1878 (1990); and Weiss, Science, 254:1292 (1991).

The isolated nucleic acids provided herein also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. A DNA polymerase can be used to extend the oligonucleotides, resulting in a single, double-stranded nucleic acid molecule per oligonucleotide pair, which then can be ligated into a vector.

A SUR2A polypeptide can have a mutant amino acid sequences shown in SEQ ID NOs:4, 5, 13, 15, and 17. SUR2A polypeptides of the invention can have at least 95% sequence identify to the reference (wild-type) SUR2A polypeptide sequence shown, for example, in SEQ ID NO:3 or 11 (e.g., GenBank Accession No. NP_{—}005682.1). SUR2A polypeptides of the invention are polypeptides that, but for the mutation, would assembly with the other subunits (Kir6.1 and Kir6.2) and transport potassium at or near levels of that of the reference polypeptide. The term “substantially pure” with respect to a naturally-occurring SUR2A polypeptide refers to a polypeptide that has been separated from cellular components by which it is naturally accompanied, such that it is at least 60% (e.g., 70%, 80%, 90%, 95%, or 99%), by weight, free from polypeptides and naturally-occurring organic molecules with which it is naturally associated. In general, a substantially pure polypeptide will yield a single major band on a non-reducing polyacrylamide gel.

A substantially pure polypeptide provided herein can be obtained by, for example, extraction from a natural source (e.g., heart tissue), chemical synthesis, or by recombinant production in a host cell. To recombinantly produce a SUR2A polypeptide or a Kir6.2 polypeptide, a nucleic acid encoding SUR2A or Kir6.2 can be ligated into an expression vector and used to transform a prokaryotic (e.g., bacteria) or eukaryotic (e.g., insect, yeast, or mammal) host cell. In general, nucleic acid constructs can include a regulatory sequence operably linked to a nucleic acid encoding a SUR2A or Kir6.2 polypeptide. Regulatory sequences (e.g., promoters, enhancers, polyadenylation signals, or terminators) do not typically encode a gene product, but instead affect the expression of a nucleic acid sequence.

A construct can include a tag sequence designed to facilitate subsequent manipulations of the expressed nucleic acid sequence (e.g., purification, localization, etc.). Tag sequences, such as green fluorescent protein (GFP), glutathione S-transferase (GST), six histidine (His₆), c-myc, hemagglutinin, or Flag™ tag (Kodak) sequences are typically expressed as a fusion with the polypeptide encoded by the nucleic acid sequence. Such tags can be inserted in a nucleic acid sequence such that they are expressed anywhere along an encoded polypeptide including, for example, at either the carboxyl or amino termini. The type and combination of regulatory and tag sequences can vary with each particular host, cloning or expression system, and desired outcome. A variety of cloning and expression vectors containing combinations of regulatory and tag sequences are commercially available. Suitable cloning vectors include, without limitation, pUC18, pUC19, and pBR322 and derivatives thereof (New England Biolabs, Beverly, Mass.), and pGEN (Promega, Madison, Wis.). Additionally, representative prokaryotic expression vectors include, without limitation, pBAD (Invitrogen, Carlsbad, Calif.), the pTYB family of vectors (New England Biolabs), and pGEMEX vectors (Promega); representative mammalian expression vectors include, without limitation, pTet-On/pTet-Off(Clontech, Palo Alto, Calif.), pIND, pVAX1, pCR3.1, pcDNA3.1, pcDNA4, or pUni (Invitrogen), and pCI or pSI (Promega); representative insect expression vectors include, without limitation, pBacPAK8 or pBacPAK9 (Clontech), and p2Bac (Invitrogen); and representative yeast expression vectors include, without limitation, MATCHMAKER (Clontech) and pPICZ A, B, and C (Invitrogen).

In bacterial systems, Escherichia coli can be used to express SUR2A or Kir6.2 polypeptides. For example, BL-21 cells can be transformed with a pGEX vector containing a nucleic acid sequence encoding a SUR2A or Kir6.2 polypeptide. The transformed bacteria can be grown exponentially and then stimulated with isopropylthiogalactopyranoside (IPTG) prior to harvesting. In general, the SUR2A-GST or Kir6.2-GST fusion polypeptides produced from a pGEX expression vector can be purified from lysed cells by adsorption to glutathione-agarose beads followed by elution in the presence of free glutathione. The pGEX vectors can be designed to include thrombin or factor Xa protease cleavage sites so that the expressed SUR2A or Kir6.2 polypeptide can be released from the GST moiety.

In eukaryotic host cells, a number of viral-based expression systems can be utilized to express SUR2A or Kir6.2 polypeptides and mutants thereof. A nucleic acid encoding a SUR2A or Kir6.2 polypeptide or mutant thereof can be cloned into, for example, a baculoviral vector such as pBlueBac (Invitrogen) and then used to co-transfect insect cells such as Spodoptera frugiperda (Sf9) cells with wild-type DNA from Autographa californica multinuclear polyhedrosis virus (AcMNPV). Recombinant viruses producing a polypeptide provided herein can be identified by standard methodology. Alternatively, a nucleic acid encoding a SUR2A or Kir6.2 polypeptide can be introduced into a SV40, retroviral, or vaccinia based viral vector and used to infect suitable host cells.

Eukaryotic cell lines that stably express SUR2A or Kir6.2 polypeptides and mutants thereof can be produced using expression vectors with the appropriate control elements and a selectable marker. For example, the eukaryotic expression vector pCR3.1 (Invitrogen) and p91023(B) (see Wong et al., Science (1985) 228:810-815) or modified derivatives thereof are suitable for expression of SUR2A or Kir6.2 polypeptides or mutants thereof in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of the expression vector into cells by electroporation, lipofection, calcium phosphate or calcium chloride co-precipitation, DEAE dextran, or other suitable transfection method, stable cell lines can be selected, e.g., by antibiotic resistance to G418, kanamycin, or hygromycin. Alternatively, amplified sequences can be ligated into a eukaryotic expression vector such as pcDNA3 (Invitrogen) and then transcribed and translated in vitro using wheat germ extract or rabbit reticulocyte lysate.

SUR2A or Kir6.2 polypeptides and mutants thereof can be purified by known chromatographic methods including ion exchange and gel filtration chromatography. See, for example, Caine et al., Protein Expr. Purif. (1996) 8(2):159-166. SUR2A polypeptides also can be “engineered” to contain a tag sequence describe herein that allows the polypeptide to be purified (e.g., captured onto an affinity matrix). In addition, immunoaffinity chromatography can be used to purify SUR2A or Kir6.2 polypeptides or mutants thereof.

Identifying Individuals with K_{ATP} Channel Defects

Individuals having defective cardiac K_{ATP} channels can be identified using a number of different methods. For example, individuals can be screened directly for a mutation in a SUR2A polypeptide of a K_{ATP} channel or for a polymorphism in a Kir6.2 polypeptide. Individuals having a mutation in a nucleic acid encoding a SUR2A or a Kir6.2 polypeptide can have an increased risk of cardiac disease. Cardiac diseases can include a variety of cardiomyopathies and/or rhythm disturbances such as Long QT Syndrome, ventricular arrhythmias (e.g., ventricular tachycardia) and atrial arrhythmias (e.g., atrial fibrillation).

Mutations in the nucleic acid encoding a SUR2A or Kir6.2 polypeptide can be detected, for example, by sequencing exons, introns, 5′ untranslated sequences, or 3′ untranslated sequences, by performing allele-specific hybridization, allele-specific restriction digests, mutation specific polymerase chain reactions (MSPCR), by single-stranded conformational polymorphism (SSCP) detection (Schafer et al., 1995, Nat. Biotechnol., 15:33-39), denaturing high performance liquid chromatography (DHPLC, Underhill et al., 1997, Genome Res., 7:996-1005), infrared matrix-assisted laser desorption/ionization (IR-MALDI) mass spectrometry (WO 99/57318), and combinations of such methods.

Genomic DNA generally is used in the analysis of SUR2A mutants or Kir6.2 polymorphisms, although mRNA also can be used. Genomic DNA is typically extracted from a biological sample such as blood, but can be extracted from other biological samples including tissue samples (e.g., from biopsied cardiac tissue). Routine methods can be used to extract genomic DNA from a blood or tissue sample, including, for example, phenol extraction. Alternatively, genomic DNA can be extracted with kits such as the QIAamp® Tissue Kit (Qiagen, Chatsworth, Calif.), Wizard® Genomic DNA purification kit (Promega) and the A.S.A.P.™ Genomic DNA isolation kit (Boehringer Mannheim, Indianapolis, Ind.). In some cases, an amplification step is performed prior to detecting the mutation. For example, the nucleic acid encoding a SUR2A or a Kir6.2 polypeptide can be amplified and then directly sequenced. Dye primer sequencing can be used to increase the accuracy of detecting heterozygous samples.

Hybridization also can be used to detect sequence mutations. See, for example, Stoneking et al., 1991, Am. J. Hum. Genet., 48:370-382; and Prince et al., 2001, Genome Res., 11:152-162. In practice, samples of DNA or RNA from one or more individuals can be amplified using pairs of primers and the resulting amplification products can be immobilized on a substrate (e.g., in discrete regions). Hybridization conditions can be selected such that an oligonucleotide specifically binds to a sequence of interest, e.g., the mutant nucleic acid sequence. Such hybridizations typically are performed under high stringency as some sequence mutations include only a single nucleotide difference. High stringency conditions can include the use of low ionic strength solutions and high temperatures for washing. For example, nucleic acid molecules can be hybridized at 42° C. in 2×SSC (0.3 M NaCl/0.03 M sodium citrate) with 0.1% sodium dodecyl sulfate (SDS) and washed in 0.1×SSC (0.015 M NaCl/0.0015 M sodium citrate), 0.1% SDS at 65° C. Hybridization conditions can be adjusted to account for unique features of the nucleic acid molecule, including length and sequence composition. Probes can be labeled (e.g., fluorescently or with biotinylation) to facilitate detection.

For nucleotide sequence mutations that introduce a restriction site, restriction digest(s) with the appropriate restriction enzyme(s) can differentiate the wild-type and mutant SUR2A alleles or the wild-type and polymorphic Kir6.2 alleles. For mutations that do not alter a common restriction site, mutagenic primers can be designed that introduce a restriction site when the mutation is present or when a wild-type allele is present. A portion of a nucleic acid molecule encoding SUR2A or Kir6.2 can be amplified using the mutagenic primer and a wild-type primer, followed by digest with the appropriate restriction endonuclease.

Certain mutations, such as insertions or deletions of one or more nucleotides, can change the size of a DNA fragment encompassing a mutation. The insertion or deletion of nucleotides can be assessed by amplifying the region encompassing the mutation and determining the size of the amplified products in comparison with size standards. For example, a region of a nucleic acid encoding a SUR2A polypeptide or regulating expression of a SUR2A polypeptide or a region of a nucleic acid encoding a Kir6.2 polypeptide can be amplified using a primer set from either side of the mutation. One of the primers is typically labeled, for example, with a fluorescent moiety, to facilitate sizing. The amplified products can be electrophoresed through acrylamide gels with a set of size standards that are labeled with a fluorescent moiety that differs from the primer.

In some embodiments, PCR conditions and primers can be developed that amplify a product only when the mutation is present or only when the mutation is not present (MSPCR or allele-specific PCR). For example, patient DNA and a control can be amplified separately using either a wild-type primer or a primer specific for the mutation. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products can be detected with each set of primers. Patient samples containing solely the wild-type allele would have amplification products only in the reaction using the wild-type primer. Similarly, patient samples containing solely the mutant allele can have amplification products only in the reaction using the primer containing the mutant sequence. Allele-specific PCR also can be performed using allele-specific primers that introduce priming sites for two universal energy-transfer-labeled primers (e.g., one primer labeled with a green dye such as fluoroscein and one primer labeled with a red dye such as sulforhodamine). Amplification products can be analyzed for green and red fluorescence in a plate reader. See, Myakishev et al., 2001, Genome, 11(1):163-169.

Mismatch cleavage methods also can be used to detect differing sequences by PCR amplification, followed by hybridization with the wild-type sequence and cleavage at points of mismatch. Chemical reagents, such as carbodiimide or hydroxylamine and osmium tetroxide can be used to modify mismatched nucleotides to facilitate cleavage.

Alternatively, mutants or variants can be detected by antibodies that have specific binding affinity for mutant SUR2A polypeptides or polymorphic Kir6.2 polypeptides. Mutant SUR2A or Kir6.2 polypeptides can be produced in various ways, including recombinantly, as discussed above. Host animals such as rabbits, chickens, mice, guinea pigs, and rats can be immunized by injection of, for example, a SUR2A mutant polypeptide. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Polyclonal antibodies are heterogeneous populations of antibody molecules that are contained in the sera of an immunized or exposed animal. Monoclonal antibodies, which are homogeneous populations of antibodies to a particular antigen, can be prepared using a SUR2A mutant polypeptide or a Kir6.2 polymorphic polypeptide and standard hybridoma technology. In particular, monoclonal antibodies can be obtained by any technique that provides for the production of antibody molecules by continuous cell lines in culture such as described by Kohler et al., Nature, 256:495 (1975), the human B-cell hybridoma technique (Kosbor et al., Immunology Today, 4:72 (1983); Cole et al., Proc. Natl. Acad. Sci. USA, 80:2026 (1983)), and the EBV-hybridoma technique (Cole et al., Monoclonal Antibodies and Cancer Therapy, Alan R. Liss, Inc., pp. 77-96 (1983)). Such antibodies can be of any immunoglobulin class including IgG, IgM, IgE, IgA, IgD and any subclass thereof. The hybridoma producing the monoclonal antibodies of the invention can be cultivated in vitro and in vivo. Antibodies (monoclonal or polyclonal) having the ability to bind mutant SUR2A polypeptides or polymorphic Kir6.2 polypeptides but that do not bind wild-type SUR2A polypeptide or wild-type Kir6.2 polypeptide are desirable.

Antibody fragments that have specific binding affinity for a SUR2A mutant polypeptide or a Kir6.2 polypeptide can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., Science, 246:1275 (1989). Once produced, antibodies or fragments thereof are tested for recognition of SUR2A mutant polypeptides or Kir6.2 polymorphic polypeptides by standard immunoassay methods including ELISA techniques, radioimmunoassays and Western blotting. See, Short Protocols in Molecular Biology, Ch. 11, Green Publishing Associates and John Wiley & Sons, edited by Ausubel et al., 1992.

In addition to directly screening individuals for mutations in the nucleic acid encoding a SUR2A polypeptide or in the nucleic acid encoding a Kir6.2 polypeptide, an individual may present with another symptom that may be associated with or antagonized by a defect in K_{ATP} channeling. For example, an individual may be diagnosed and undergoing conventional treatment for sepsis or for hypertension. Without limitation, conventional treatments include, for example, antibiotics for the treatment of sepsis and anti-hypertensive drugs for the treatment of hypertension. If such an individual is not responding to the conventional treatments, that individual may have a deficiency in K_{ATP} channeling (e.g., a mutation in the nucleic acid encoding a SUR2A or a Kir6.2 polypeptide). Thus, individuals can be screened at the time of diagnosis of sepsis or hypertension or subsequent to diagnosis if they are not responding to conventional treatment. Such individuals can be screened as described herein. If individuals having sepsis and/or hypertension are additionally diagnosed with a deficiency in K_{ATP} channeling, those individuals can be treated with potassium channel openers (PCOs) as discussed herein in addition to continuing the conventional treatments the individual may receive.

Individuals also may present with vague indications of cardiac issues. For example, catecholamine induction is routinely used for cardiological evaluation of an individual. If the outcome of catecholamine induction is poor (e.g., a diagnosis of unidentified cardiac issues), the individual can be screened for a deficiency in KATP channeling and/or mutations in nucleic acid encoding a SUR2A polypeptide or regulating the expression of a SUR2A polypeptide. Such screening methods are described herein.

Treating Individuals with K_{ATP} Channel Defects

Methods for treating an individual for a deficiency in cardiac K_{ATP} channeling are provided herein. First, individuals having a deficiency in cardiac K_{ATP} channeling can be identified. Identifying such individuals can be performed as described herein. Individuals having a deficiency in cardiac K_{ATP} channeling can be treated by administering an effective dose of a potassium channel opener (PCO) to the individual. Representative PCOs include, for example, WAY-133537; ZD6169; Celikalim; 1,4-benzoxazine skeleton into 1,4-benzothiazine; 1,2,3,4-tetrahydroquinoline; 1,2,3,4-tetrahydroquinoxaline; indoline; 1,5-benzoxazepine; diazoxide; minoxidil; and lemakalim (BRL38227). See also, for example, Fozard & Manley, 2001, Potassium Channel Openers, Prog. Respir. Res., 31:77-80; and U.S. Pat. No. 5,972,894.

One or more PCOs can be incorporated into pharmaceutical compositions suitable for administration. Such compositions typically comprise the PCO and a pharmaceutically acceptable carrier. As used herein, “pharmaceutically acceptable carrier” is intended to include solvents, dispersion media, coatings, antibacterial and anti-fungal agents, isotonic and absorption delaying agents, and the like that are compatible with pharmaceutical administration. The use of such media and agents for pharmaceutically active substances is well known in the art. Except insofar as any conventional media or agent is incompatible with the active compound, use thereof in the compositions is contemplated. Supplementary active compounds also can be incorporated into the compositions.

A pharmaceutical composition can be formulated to be compatible with its intended route of administration. Examples of routes of administration include, without limitation, parenteral, e.g., intravenous, intradermal, subcutaneous, oral (e.g., ingestion or inhalation), transdermal (topical), and transmucosal administration. Solutions or suspensions used for parenteral, intradermal, or subcutaneous application can include the following components: a sterile diluent such as water for injection, saline solution (e.g., phosphate buffered saline (PBS)), fixed oils, a polyol (for example, glycerol, propylene glycol, and liquid polyetheylene glycol, and the like), glycerine, or other synthetic solvents; antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The proper fluidity can be maintained, for example, by the use of a coating such as lecithin, by the maintenance of the required particle size in the case of dispersion and by the use of surfactants. In some cases, isotonic agents such as sugars, polyalcohols (e.g., mannitol or sorbitol), and sodium chloride can be included in a composition. Prolonged administration of an injectable composition can be brought about by including an agent that delays absorption. Such agents include, for example, aluminum monostearate and gelatin. The parenteral preparation can be enclosed in ampoules, disposable syringes or multiple dose vials made of glass or plastic.

Oral compositions generally include an inert diluent or an edible carrier. Oral compositions can be liquid, or can be enclosed in gelatin capsules or compressed into tablets. Pharmaceutically compatible binding agents, and/or adjuvant materials can be included as part of an oral composition. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Transmucosal administration can be accomplished through the use of nasal sprays or suppositories. For transdermal administration, the active compounds are formulated into ointments, salves, gels, or creams as generally known in the art.

After a compound is administered to the individual, the individual can be monitored to evaluate whether or not the PCOs are improving K_{ATP} channeling. Individuals can be monitored using several methods. For example, the condition of individuals presenting with sepsis or hypertension, for example, may begin improving and those individuals may begin responding to the conventional treatments. In addition, catecholamine induction can be performed on an individual receiving PCOs. An improvement in the outcome (e.g., better than poor) indicates responsiveness to the PCOs.

Certain individuals having a mutation in the nucleic acid encoding a SUR2A polypeptide may not respond to one or more PCOs. Although not bound by any particular mechanism, PCO-resistance may be due to a reduction in binding of the PCO by a SUR2A polypeptide due to a mutation. In these cases, other PCOs can be administered to the individual, and the individual monitored for responsiveness. If PCOs do not seem to be effective, those individuals can be administered a Ca²⁺ channel blocker. The same ingredients described herein for a pharmaceutical composition containing one or more PCOs can be used to make a composition containing one or more Ca²⁺ channel blockers. In addition, the same routes of administration described herein for a pharmaceutical composition containing one or more PCOs can be sued to administer a composition containing one or more Ca²⁺ channel blockers.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### EXAMPLES

### Part I. Identification of Mutations in SUR2A

Example 1—Materials & Methods

Mutational analysis. Mutation scans of K_{ATP} channel genes were carried out in individuals, predominantly of European descent, with idiopathic dilated cardiomyopathy who gave informed consent with approval form the Mayo Clinic Institutional Review Board. According to established diagnostic criteria, individuals had left ventricular dimensions>95^{th} percentile indexed for body surface area and age and left ventricualr ejection fractions<50% as determined by echocardiography. Primers for exon-specific PCR amplification were designed using the OLIGO v6.51 Primer Analysis Software (National Biosciences) and the WAVEMAKER version 4.0.32 Software (Transgenomic). Variations in the sequence were identified in PCR amplified DNA fragments by denaturing high-performance liquid chromatography heteroduplex analysis (WAVE DNA Fragment Analysis System, Transgenomic). Fragments that formed heteroduplexes were amplified by PCR and cycle-sequenced using a ThermoSequence kit (Amersham Life Sciences). To confirm mutation, mutated alleles were isolated on 1× Mutation Detection Enhancement gels (FMC Bioproducts), reamplified and sequenced. Healthy blood donors served as controls.

Homology modeling and molecular dynamics simulation. The three-dimensional model of SUR2A NBD2 was generated using the homology modeling program MODELLER 6 using as a template the crystal structure of the ATPase domain in the human TAP1 protein with ADP in the binding pocket (PDB code 1JJ7) identified by the FASTA search (Gaudet & Wiley, 2001, EMBO J, 20:4964-72). The initial atomic NBD2 three-dimensional model was refined by energy minimizations and a 2.0-ns (1.0-fs time step) molecular dynamics simulation using the AMBER 5.0/6.0 program with a second generation force field (parm96.dat) according to established protocols (Pang, 2001, Proteins, 45:183-9).

Cloning of K_{ATP} channel subunits and purification of SUR2A ATPase. cDNAs encoding SUR2A and Kir6.2 were subcloned into the expression vector pcDNA3.1. PCR-based site-directed mutagenesis was carried out using primers incorporating identified mutations (QuickChange, Stratagene). The constructs were sequenced to verify mutations and rule out additional changes in sequence. Soluble SUR2A NBD2 (Thr1220-Thr1546) was purified by overexpressing a fusion protein construct containing the maltose binding protein (MBP) used for affinity chromatography on an amylase resin in 600 mM NaCl, 1 mM EDTA, 20 mM Tris, 1 mM dithiothreitol, and 10% glycerol (Zingman et al., 2002, J. Biol. Chem., 277:14206-10 and references therein).

Enzymology and nucleotide binding. ATPase activity of purified NBD2 was measured in the presence of Mg²⁺, as Pᵢ generation detected spectrophotometrically at 360 nm with the EnzChek Phosphate Assay Kit or by monitoring conversion of [γ-³²P]ATP using thin layer chromatography and quantified it with Phosphorlmager and ImageQuant software (Molecular Dynamics) (Zingman et al., 2002, supra). The initial kinetics of ATP hydrolysis was captured by stopped-flow spectroscopy with instantaneous mixing of purified polypeptide and substrate on an SX. 18MV spectrometer (Applied Photophysics). Binding of fluorescent ATP analog TNP-ATP to purified NBD2 constructs was determined by fluorescence spectrophotometry (QuantaMaster, PTI) at excitation and emission wavelengths of 410 nm and 540 nm, respectively.

Surface channel subunit expression. cRNA encoding wild-type or mutant SUR2A (6 ng), tagged with an extracellular hemagglutinin epitope, with cRNA encoding Kir6.2 (2 ng) were injected into Xenopus laevis oocytes (Schwappach et al., 2000, Neuron, 26:155-67). After 72 hrs, oocytes were labeled at 4° C. with 0.4 μg ml⁻¹ monoclonal antibody to hemagglutinin and horseradish peroxidase-coupled secondary antibody. Chemiluminescence at the plasmalemma of individual oocytes, generated from channel proteins that trafficked to the surface, was captured in Power Signal ELISA solution (Pierce) and quantified using a plate reader (Lab Systems).

Electrophysiology. Human embryonic kidney (HEK293) cells cultured at 5% CO₂ in Dulbecco's modified Eagle medium with 10% fetal calf serum and 2 mM glutamine were co-transfected with 0.5 μg of reporter green fluorescent protein and 6 μg of total plasmid DNA (cDNAs encoding wild-type of mutant SUR2A and Kir6.2 at a 5:1 ratio) using 15 μl of Fugene (Roche). The patch-clamp technique was applied using patch electrodes filled with 140 mM KCl, 1 mM CaCl₂, 1 mM MgCl₂ and 5 mM HEPES buffer (pH 7.3) and the cells were superfused with 140 mM KCl, 1 mM MgCl₂, 5 mM EGTA, and 5 mM HEPES-KOH buffer (pH 7.3). KAPT channel activity was measured at 31±1° C. using a temperature controller (HCC-100A; Dagan) (Zingman et al., 2001, Neuron, 31:233-245).

Kinetics of SUR2A ATPase.

In the ATPase catalytic cycle, E+ATP [symbol] E*·ATP [symbol] E**·ADP·Pᵢ [symbol] E***·ADP+Pᵢ [symbol] E+ADP+Pᵢ, E is SUR2A and stars indicate distinct conformations during Mg²⁺-dependent ATP hydrolysis. Transitions are bi-directional, with the exception of the Pᵢ liberation step (k₃), which is irreversible in SUR2A NBD2 constructs. Therefore, the steady-state rate equation of the ATPase reaction was derived by the King-Altman method as:

$$\begin{matrix}\frac{{E}_{t}}{v} = \frac{{slope}_{1}}{[ ATP ]} + \frac{[ ADP ] {slope}_{1}}{[ ATP ] {K}_{ADP}} + \frac{1}{{v}_{max}} \\ & ( Equation 1 ) \\ \begin{matrix}{slope}_{1} = \frac{1}{{k}_{1}} + \frac{{K}_{ATP}}{{k}_{2}} + \frac{{K}_{ATP}}{{k}^{3} {K}_{ADP \cdot Pi}} and \frac{1}{{v}_{max}} \\ = \frac{1}{{k}_{2}} + \frac{1}{{k}_{3}} + \frac{1}{{k}^{3} {K}_{ADP \cdot Pi}} + \frac{1}{{k}_{4}}\end{matrix} & ( Equation 2 )\end{matrix}$$

where Eₜ is total enzyme amount, ν is the reaction rate, slope₁ is the slope of double-reciprocal dependence of the reaction rate ν on ATP (at [ADP]=0), νₘₐₓ is the maximal reaction rate, K_{ATP}=k₋₁/k₁, K_{ADP·Pi}=k₂/k₋₂, and K_{ADP}=k₄/k₋₄.

The ADP dissociation constant, K_{ADP}, was determined from spectrometric measurement of steady-state NBD2 ATPase activity at [ADP]=O with variable [ATP]. Equation 1 thus can be simplified:
Eₜ/ν=slope₁/[ATP]+1/νₘₐₓ, (Equation 3)
where slope₁ and νₘₐₓ are assessable for experimental determination. At variable [ADP], with constant [ATP] ([ATP]_{c}), Equation 1 is a linear function of [ADP]:
Eₜ/ν=[ADP]slope₁/K_{ADP}[ATP]_{c}+slope₁/[ATP]_{c}+1/νₘₐₓ (Equation 4)
where the slope of the reciprocal reaction rate: slope₂=slope₁/K_{ADP}[ATP]_{c}. Thus, K_{ADP} was defined as:
K_{ADP}=slope₁/slope₂[ATP] (Equation 5)

Kinetic rate constants of the ATPase reaction were defined using stopped-flow spectroscopy where virtually instantaneous initiation of the ATPase reaction was achieved by mixture of NBD2 constructs and MgATP (2 mM ATP, 3 mM MgCl₂). During the first catalytic cycle, the time required to reach the step of Pᵢ liberation is shorter than in subsequent reaction cycles. Therefore, the rate of reaction product accumulation in the initial pre-steady state is higher than in the following steady-state reaction. Kinetic parameters of the ATPase cycle were defined by fitting the kinetics of product accumulation with a system of differential equations with Equations 2 and 5:
d[E]/dt=−[E](k₁[ATP]+k₋₄[ADP])+[E·ATP]k₋₁+(Eₜ−[E]−[E·ATP]−[E·ADP·Pᵢ])k₄
d[E·ATP]/dt=[E]k₁[ATP]−[E·ATP](k₋₁+k₂)+[E·ADP·Pᵢ]k₋₂
d[E·ADP·Pᵢ]/dt=−[E·ADP·Pᵢ](k₋₂+k₃)+[E·ATP]k₂
d[Pᵢ]/dt=[E·ATP·Pᵢ]k₃
d[E·ADP]/dt=(Eₜ−[E]−[E·ATP]−[E·ADP·Pᵢ])k₄+[E][ADP]k₋₄ (Equation 6)
where Eₜ is the total enzyme amount. The system was solved using the Runge-Kutta algorithm. Probabilities for NBD2 ATPase conformations (E-ATP, E·ADP·Pᵢ, E·ADP) were calculated as:
P_{E·ATP}=D_{E·ATP}/(D_{E}+D_{E·ATP+D}_{E·ADP·P}ᵢ+D_{E·ADP}) (Equation 7)
P_{E·ADP·Pi}=D_{E·ADP·P}_{ᵢ}/(D_{E}+D_{E·ATP}+D_{E·ADP·P}_{ᵢ}+D_{E·ADP}) (Equation 8)
P_{E·ADP}=D_{E·ADP}/(D_{E}+D_{E·ATP}+D_{E·ADP·P}_{ᵢ}+D_{E·ADP}), (Equation 9)
where D_{E}=(k₂k₃+k₋₁k₃+k₋₁k₋₂)k₄; D_{E·ATP}=(k₃+k₋₂)k₁k₄[ATP]; D_{E·ADP·P}_{ᵢ}=k₁k₂k₄[ATP]; D_{E·ADP}=k₁k₂k₃[ATP]+(k₂k₃+k₋₁k₃+k₋₁k₋₂)k₋₄[ADP].

Lifetimes of conformations in the ATPase cycle were defined as reciprocal values of the sum of rates leading away from particular conformations:

$${τ}_{E \cdot ATP} = 1 / {k}_{- 1} + {k}_{2} ; {τ}_{E \cdot ADP \cdot {P}_{i}} = 1 / {k}_{- 2} + {k}_{3} ; {τ}_{E \cdot ADP} = 1 / {k}_{4} , ( {k}_{- 3} = 0 ) \qquad ( Equation 10 )$$

Example 2—Results

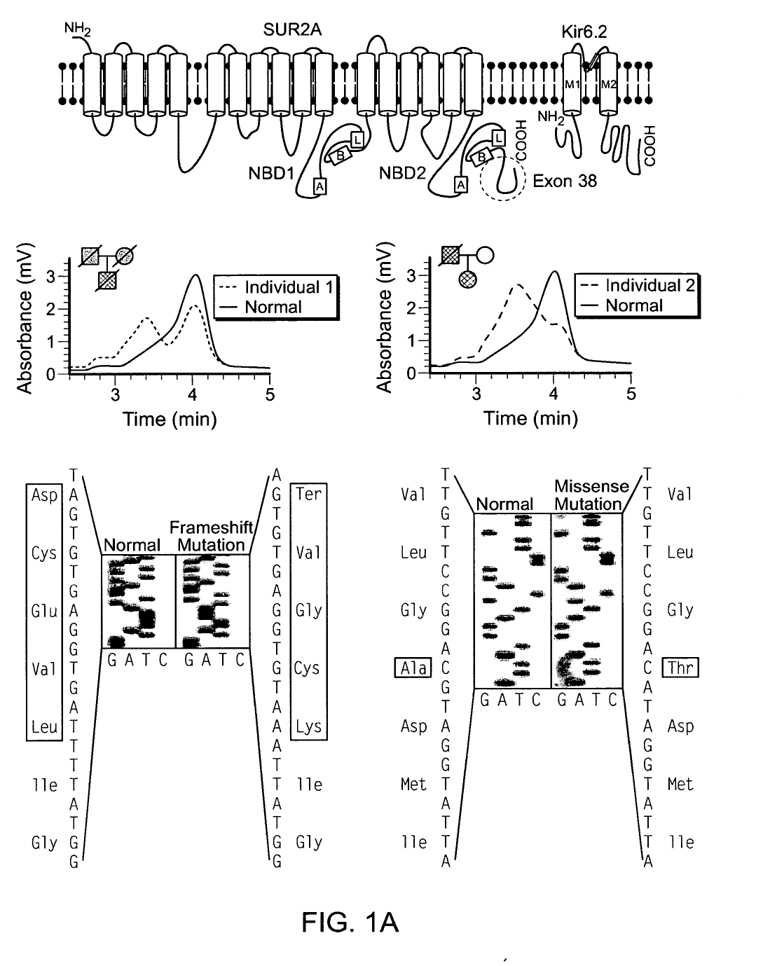

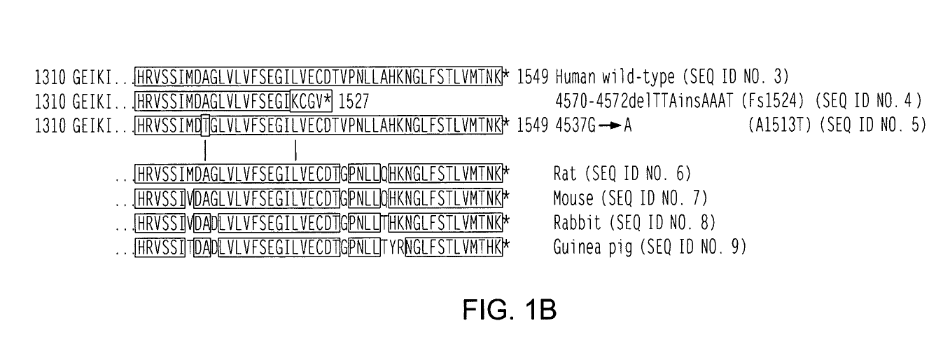

Scans for mutation in genomic DNA in a cohort of 323 individuals with idiopathic dilated cardiomyopathy identified two heterozygous mutations in exon 38 of ABCC9, which encodes the C-terminal domain of SUR2A specific to the cardiac splice variant of the regulatory K_{ATP} channel subunit (FIG. 1A,B). Both individuals with mutations in ABCC9 had severely dilated hearts with compromised contractile function and rhythm disturbances (Table 1). DNA sequencing of one mutated allele identified a 3-bp deletion and 4 bp insertion mutation (4570-4572delTTAinsAAAT), causing a frameshift at Leu1524 and introducing four anomalous terminal residues followed by a premature stop codon (Fs1524; FIG. 1A). The second mutated allele harbored a missense mutation (4537G→A) causing the amino acid substitution A1513T (FIG. 1A). The identified frameshift and missense mutations occurred in evolutionarily conserved domains of the C terminus of SUR2A (FIG. 1B) and neither mutation was present in 500 unrelated control individuals.

TABLE 1
Summary of clinical phenotypes

| | | Family | Gender, | | | | | |
|---|---|---|---|---|---|---|---|---|
| | | history | Age at | | | | | |
| | ABCC9 | of | diagnosis | LVEDD | EF | Coronary | Cardiac | |
| Individual | mutation | DCM | (yr) | (mm)^{a} | (%)^{b} | angiography | rhythm^{c} | Outcome |
| 1 | Frameshift | No | Male | 65 (55) | 23 | Normal | Ventricular | Death |
| | (Fs1524) | | 55 | | | | tachycardia | from |
| | | | | | | | | heart |
| | | | | | | | | failure at |
| | | | | | | | | 60 |
| 2 | Missense | Yes | Female | 89 (52) | 15 | Normal | Ventricular | Under |
| | (A1513T) | | 40 | | | | tachycardia | intensive |
| | | | | | | | | therapy |
| Father of 2 | | | Male | 81 (53) | 13 | Normal | Ventricular | Death |
| | | | 54 | | | | tachycardia | from |
| | | | | | | | | heart |
| | | | | | | | | failure at |
| | | | | | | | | 55 |

^{a}LVEDD, left ventricular end-diastolic dimension. The 95^{th} percentile values are given in parenthesis;
^{b}EF, left ventricular ejection fraction;
^{c}Cardiac rhythm was documented by electrocardiographic monitoring.

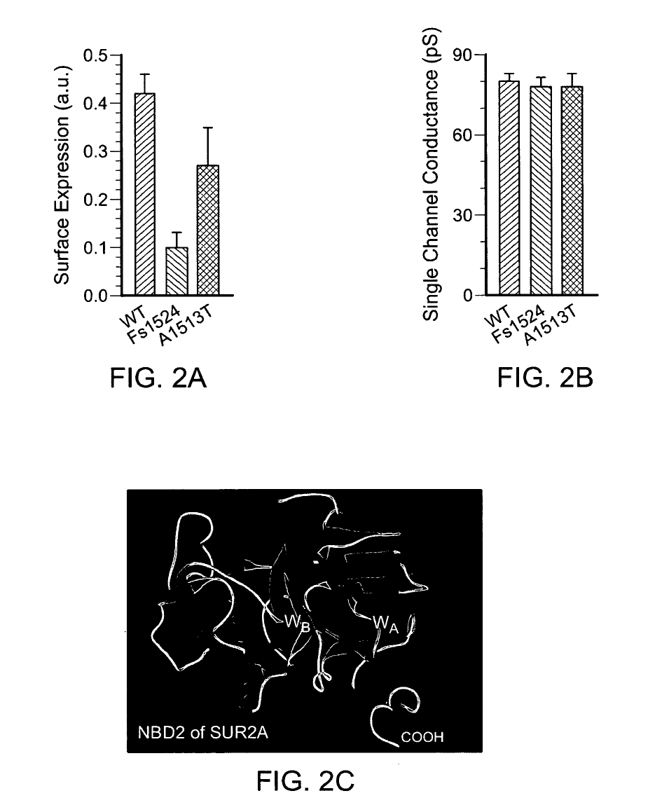

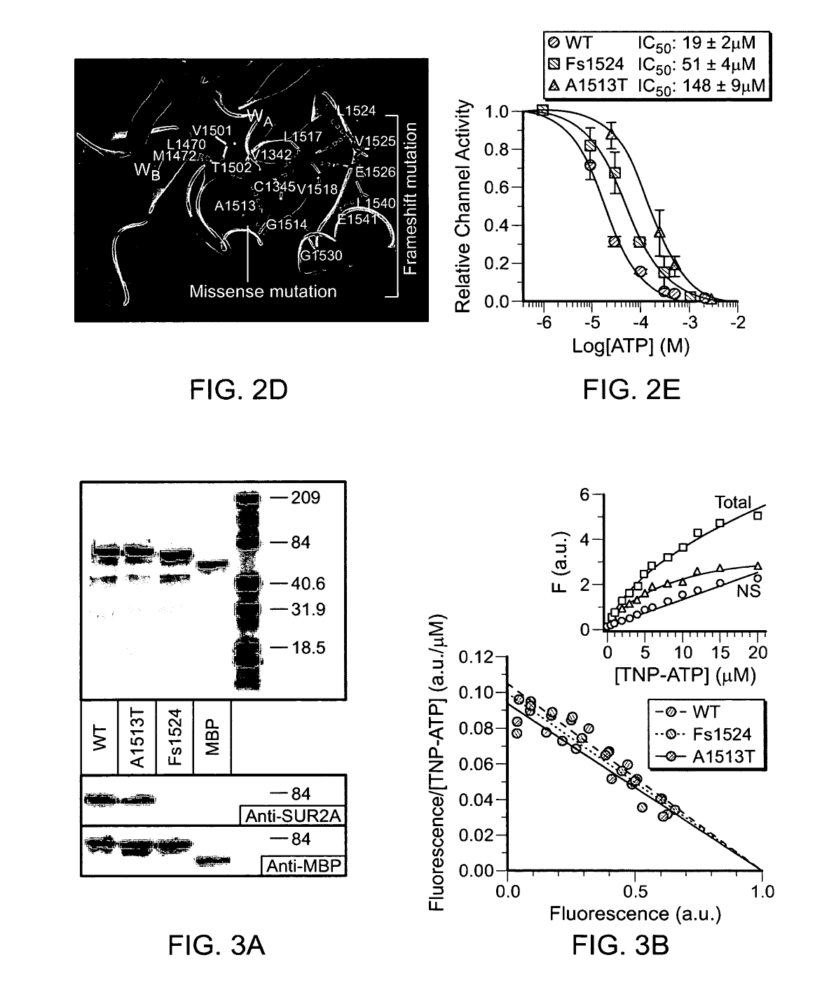

The C-terminus of SUR polypeptides contributes to K_{ATP} channel trafficking, and Fs1524 and A1513T SUR2A mutants, reconstituted with Kir6.2, had reduced expression in the plasma membrane (FIG. 2A). Yet mutant K_{ATP} channel complexes formed functional channels with intact pore properties (FIG. 2B). Structural molecular dynamics simulation showed that the residues Ala1513 and Leu1524 flank the C-terminal β-strand in close proximity to the signature Walker A motif (FIG. 2C,D), required for coordination of nucleotides in the catalytic pocket of ATP-binding cassette proteins (Walker et al., 1982, EMBO J., 1:945-51). Replacement of Ala1513 with a sterically larger and more hydrophilic threonine residue or truncation of the C terminus caused by the Fs1534 mutations would disrupt folding of the C-terminal β-strand and, thus, the tertiary organization of the adjacent second nucleotide binding domain (NBD2) pocket in SUR2A. ATP-induced K_{ATP} channel gating was aberrant in both channel mutants (FIG. 2E), suggesting that structural alterations induced by the mutations in A1513T and Fs1524 of SUR2A distorted APT-dependent pore regulation.

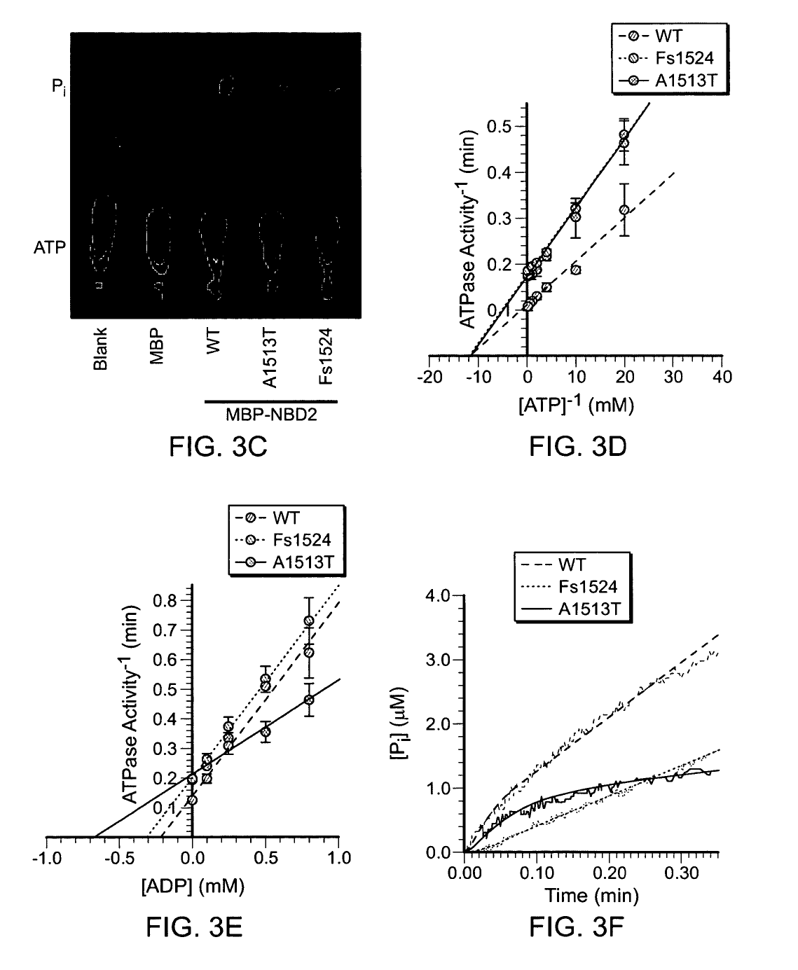

On further examination, purified wild-type and mutant NBD2 constructs had similar ATP binding but reduced ATP hydrolytic activities (FIG. 3A-C). The A1513T and Fs1524 mutations substantially diminished the maximal rate of the NBD2 ATPase reaction without altering the Michaelis-Menten constant of catalysis (FIG. 3D). A1513T reduced the product-dependent inhibition of the NBD2 ATPase more substantially than Fs1534 (FIG. 3E) but produced a less severe delay in the pre-steady state profile of product accumulation (FIG. 3F). Thus, the mutations A1513T and Fs15324 compromise ATP hydrolysis at SUR2A NBD2, generating distinct reaction kinetic defects.

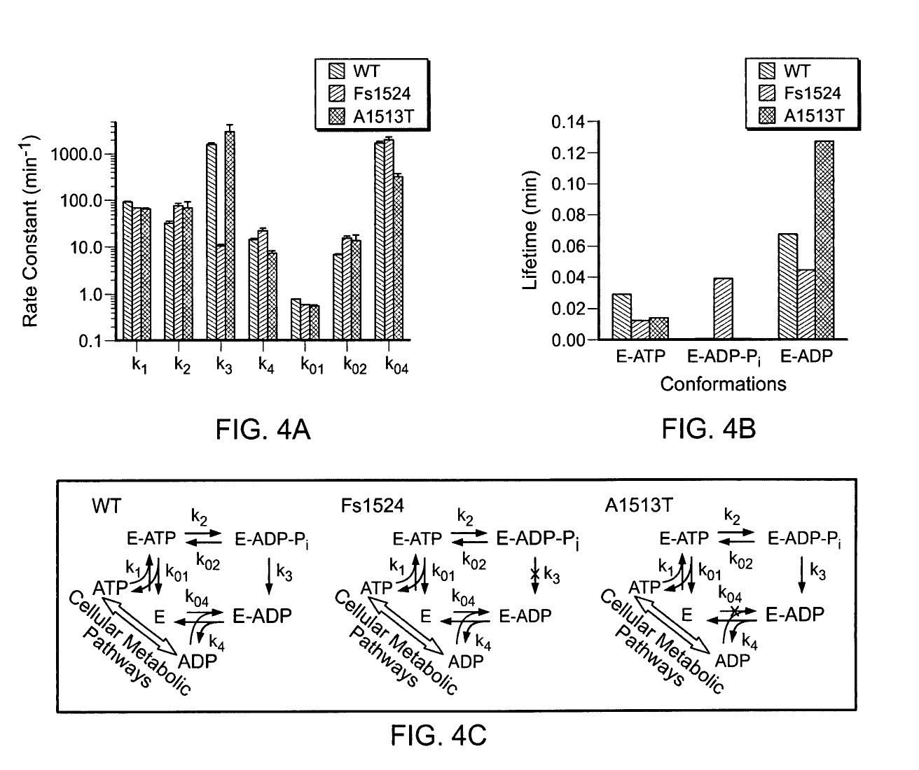

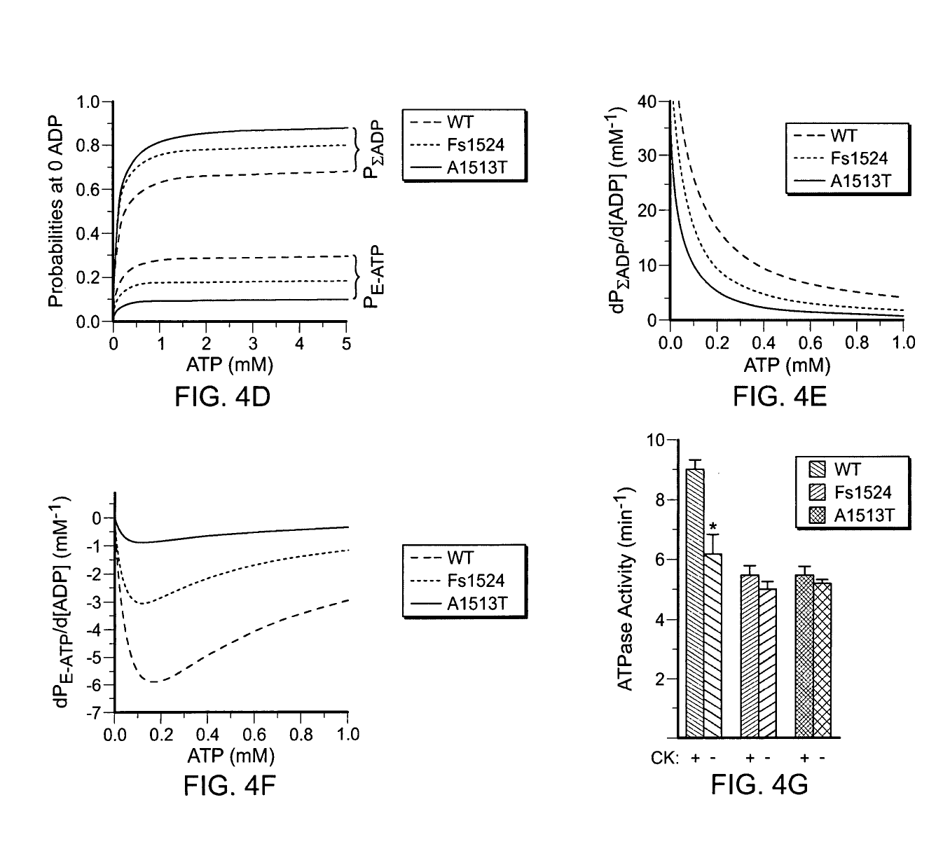

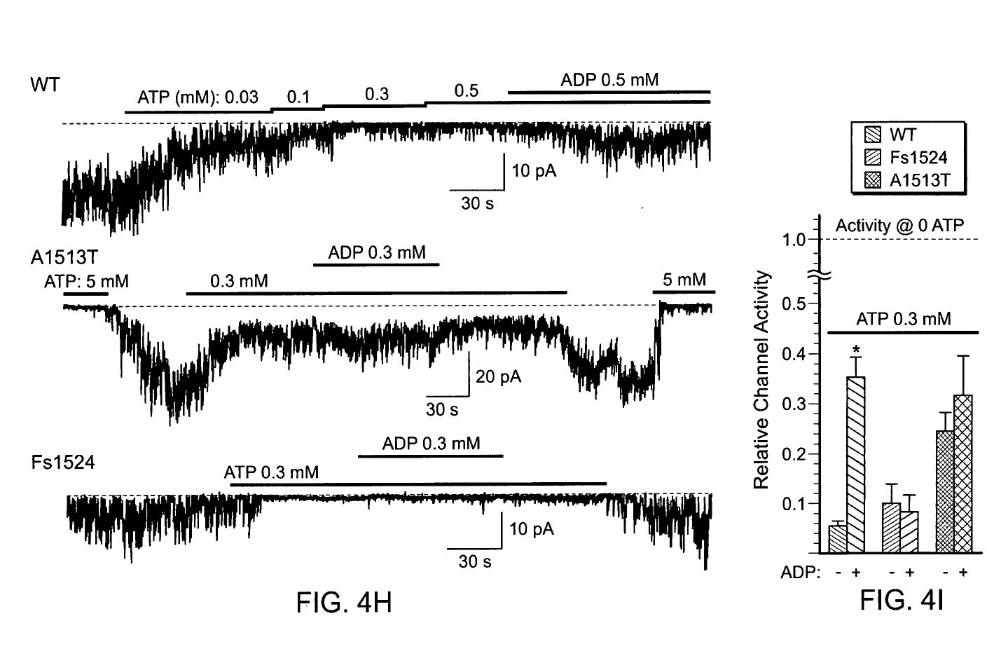

Aberrant catalytic properties in the A1513T and Fs1524 mutants translated into abnormal interconversion of discrete conformations in the NBD2 ATPase cycle (FIG. 4A). Each mutation doubled the rate constant of the SUR-ATP to SUR-ADP-Pi conversion (k2), reducing the lifetime of SUR2A in the prehydrolytic state (FIG. 4A,B). Moreover, the rate constant of the SUR-ADP-Pi to SUR-ADP transition (k3) was one hundred times lower in the Fs1524 mutant, markedly extending the lifetime of the SUR-ADP-Pi conformation and ‘jamming’ the ATPase cycle (FIG. 4A-C). Thus, in contrast to the catalytic reaction in the wild-type where the rate-limiting step is ADP dissociation (k4), the Fs1524 ATPase is characterized by rate-limiting Pᵢ dissociation (k3; FIG. 4C). In contrast, the A1513T mutation delayed the ATPase cycle in the SUR-ADP conformation, by reducing the rate constant defining ADP dissociation (k4) by a factor of 2, and reducing the ADP association rate constant (k04) by a factor of 10 (FIG. 4A-C).

The ATPase cycle in both A1513T and Fs1524 mutants was abnormally delayed in a posthydrolytic conformation, SUR-ADP-Pi or SUR-ADP. Although they had distinct patterns of lifetime distribution, each mutation diminished the likelihood that SUR2A cold adopt a prehydrolytic conformation and increased the probability of posthydrolytic conformation (FIG. 4D). Individual conformation of the SUR2A ATPase cycle have distinct impacts on K_{ATP} channel regulation, with the prehydrolytic SUR-ATP state promoting channel closure and the posthydrolytic SUR-ADP-Pi and SUR-ADP states favoring channel activation (Zingman et al., 2001, supra). Consequently, alteration in hydrolysis-driven SUR2A conformational probability induced by A1513T and Fs1524 translated into abnormal ATP sensitivity of mutant channels (FIG. 2E).

Under metabolic stress, the increase in intracellular ADP favors posthydrolytic ADP-bound conformations associated with antagonism of ATP-induced K_{ATP} channel pore inhibition (Zingman et al., 2001, supra). ADP-dependent K_{ATP} channel regulation, and thus the channel's stress responsiveness, are represented by their relative shift from prehydrolytic to posthydrolytic of the conformational probability (P) of the APase induced by changes in ADP (dP/d[ADP]). Compared to the wild-type and at any given ATP level, A1513T and Fs1524 mutants were less responsive in ADP-induced redistribution of post- (FIG. 4E) and prehyrdrolytic (FIG. 4F) conformations, Accordingly, metabolic pathways, like the creatine kinase phosphotransfer system, effectively regulated ATPase activity of wild-type but not mutant SUR2A (FIG. 4G). In fact, aberrant catalysis in mutant SUR2A generated defective K_{ATP} channel phenotypes characterized by abnormal responses to both ATP (FIG. 2E) and ADP (FIG. 4H,I) mediators o the cellular energetic state. Thus, the mutations A1513T and Fs1524 altered intrinsic catalytic properties of the SUR2A ATPase, compromising proper translation of cellular energetic signals into K_{ATP} channel-mediated membrane electrical events.

K_{ATP} channel mutations, identified in two individuals with dilated cardiomyopathy, underscore the essential role of the intrinsic enzymatic reaction in cardiac channel pore regulation. Aberrant kinetics of hydrolysis at the regulatory channel subunit, despite unaltered nucleotide binding, produced defective metabolic signal decoding. Thus, nucleotide-dependent regulation of the stress-responsive K_{ATP} channel is based not only on conventional competition between ATP and ADP at nucleotide binding domains but also on the profile of conformational interconversion driven by the catalytic cycle in the regulatory channel subunit. Traditionally linked to defects in ligand interactions, subunit trafficking, or pore conductance, human cardiac K_{ATP} channel dysfunction provoked by alteration in the catalytic module of the channel complex established a new mechanism for channelopathy. In this way, defective ion channel function confers susceptibility to dilated cardiomyopathy.

### Part II. Kir6.1 Knockout Mice in a Model of Severe Sepsis

Example 1—Materials & Methods

Kir6.1-deficient Mice. Mice deficient in vascular K_{ATP} channels (Kir6.1-KO), while maintaining normal K_{ATP} channel current in non-vascular tissues including the myocardium, were generated by targeted disruption of the KCNJ8 gene encoding the pore-forming Kir6.1 subunit and backcrossed for more than 5 generations to a C57BL/6 background (Miki et al., 2002, Nat. Med., 8:466-72). Six to eight week-old Kir6.1-KO mice were compared to age and sex matched wild-type littermate controls or to mice lacking non-vascular, including myocardial, K_{ATP} channels generated by targeted disruption of the KCNJ11 gene that encodes the Kir6.2 channel pore (Zingman et al., 2002, PNAS USA, 99:13278-83; Suzuki et al., 2001, Circ. Res., 88:570-7). All groups of mice used were selected at the time of weaning and/or genotyping by tail PCR (3-4 weeks of age), and were observed for an average of 2 weeks before experiments. Comparisons between groups were performed by analysis of variance, Student's t-tests, non-parametric or log-rank tests, using JMP software (SAS). Data are presented as mean±SEM; n refers to the sample size. P<0.05 was predetermined. All chemicals were obtained from Sigma. The Mayo Foundation Institutional Animal Care and Use Committee approved all experimental protocols.

Endotoxic Shock. The shock state of severe sepsis was modeled by administration of Escherichia coli LPS serotype 0111:B4 (Natanson et al., 1989, J. Exp. Med., 169:823-32; Suffredini et al., 1989, N. Engl. J. Med., 321:280-7). Dose-response mortality curves were obtained over a range of LPS concentrations (0.1-1000 μg/g) administered intraperitoneally in 100-200 μl sterile saline. For all other in vivo experiments, the dose of LPS was 15 μg/g. To monitor survival, mice were observed without anesthesia hourly for 24 h, every 6 h for a further 48 hours, and then daily for a total of 7 days. The effect of calcium channel blockade on LPS-induced mortality in the Kir6.1-KO was assessed by comparing survival in verapamil (5 μg/g subcutaneously every 5 h) versus saline-treated mice.

Telemetry and Electrocardiography. To continuously monitor cardiac activity and core temperature in the conscious state, telemetry devices (Data Sciences International) were implanted in the peritoneum and leads tunneled subcutaneously in a lead II configuration under isoflurane anesthesia in wild-type and Kir6.1-KO. Following recovery from surgery (24 h), signals were acquired at 2 kHz before and following LPS administration. For continuous surface electrocardiographic (ECG) recordings following anesthesia with isoflurane (1%), wild-type and Kir6.1-KO mice had continuous ECG monitoring via limb lead electrodes. Tracings were recorded for 20 min before and up to 6 h after LPS administration.

Histopathology. Light microscopy was performed on paraffin-embedded myocardial sections stained with hematoxylin-eosin from 4% formalin-fixed left ventricles (LV) taken from wild-type and Kir6.1-KO mice 4-6 h following LPS or saline vehicle administration (Hodgson et al., 2003, EMBO J, 22:1732-42). Alternatively, transmitted electron microscopy was performed on ultramicrotome-cut, lead citrate-stained, LV sections with a JEOL 1200 EXII electron microscope (Hodgson et al., 2003, supra).

In vivo Hemodynamics. Echocardiography with heart rate measurements (c256 and 15L8, Acuson) was performed in lightly sedated (1% isoflurane) wild-type and Kir6.1-KO before and 90, 180 and 360 min after LPS administration. Images were digitally acquired and stored for off-line blinded analysis. Echocardiographic measurements of LV dimensions were recorded at end diastole (EDD) and end systole (ESD) from 3 consecutive cardiac cycles using the leading edge method (Hodgson et al., 2003, supra). LV fractional shortening (% FS) was calculated as: % FS=[(EDD-ESD)/EDD]×100. Ejection time (Et) was determined from the actual pulsed-wave Doppler tracings of LV outflow by measuring the interval from the beginning of the acceleration to the end of the deceleration. The myocardial velocity of LV circumferential shortening (Vcf expressed in circumferences per s) was calculated as: Vcf=[(EDD−ESD)/EDD]Et. Blood pressure was measured under light sedation (1% isoflurane) by tail-cuff (Columbus Instruments) before and 90 min following LPS challenge.

Blood Analysis. Blood oxygen concentration, pH, lactate (iStat Corporation) and glucose (Lifescan) were measured 6 h post-LPS or post-saline. Serum TNFα levels were quantified at baseline, 90 and 180 min following LPS by ELISA (R&D Systems).

Coronary Flow. The aorta was cannulated in situ. The heart was excised, retrogradely perfused at 90 mm Hg with Krebs-Henseleit buffer (bubbled with 95% O₂/5% CO₂ at 37° C.; pH 7.4), and paced at a rate of 600 beats per min (Hodgson et al., 2003, supra). Coronary flow was continuously measured with a T106 small animal blood flow meter (Transonic Systems Incorporated). The effect of recombinant murine TNFα was assessed at least 20 min after flow stabilization. In a subset of hearts, TNFα was preceded by 10 min perfusion of 10 μM glyburide in the wild-type or followed by 1 μM verapamil in the Kir6.1-KO. Maximum vasodilation was defined as the peak increase in coronary flow observed in the wild-type in response to 10 μl of 1 mM adenosine after at least 10 min of return to baseline flow on TNFα washout.

Example 2—Results

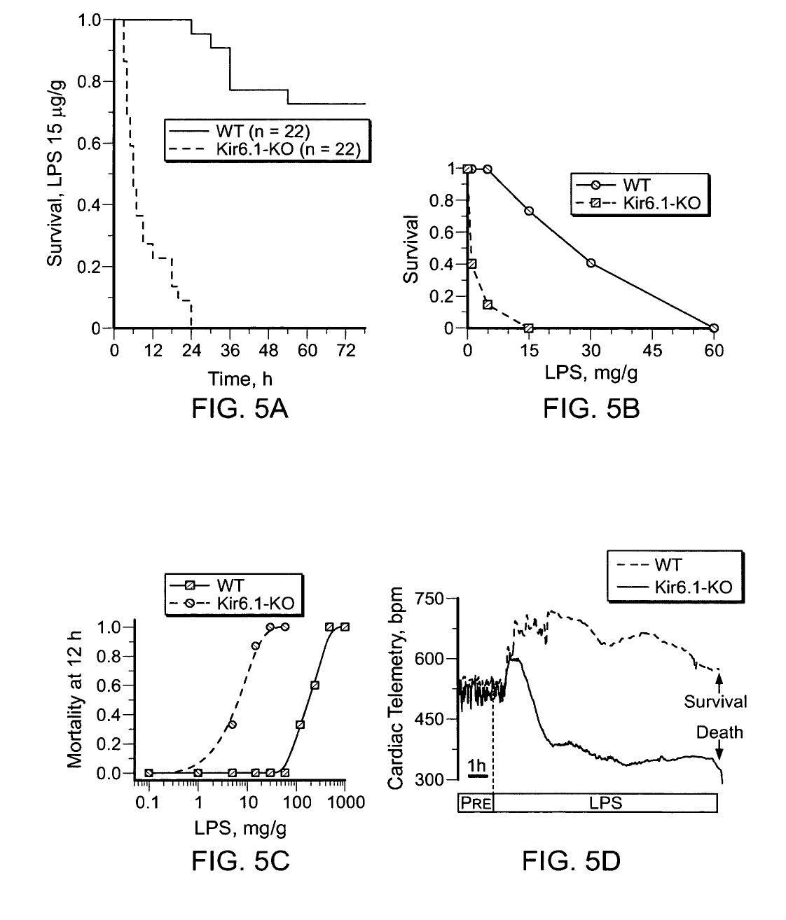

Knockout of Kir6.1 Predisposes to LPS-induced Death. The shock state of severe sepsis was simulated in mice lacking the pore-forming subunit of vascular KATP channels (Kir6.1-KO) and in wild-type littermates by the intraperitoneal administration of Escherichia coli LPS. All mice displayed decreased activity, piloerection, periocular discharge and diarrhea, typical early signs of endotoxemia. Compared to the more tolerant wild-type (6/22) or to vehicle-challenged Kir6.1-KO controls (0/10), however, Kir6.1-KO mice were predisposed to a prompt and massive mortality following 15 μg/g LPS (22/22, P<0.0001; FIG. 5A). Kir6.1-KO mice began to die within the first 3 h, with 50% death observed at 7 h and mortality in the whole cohort confirmed within 24 h (FIG. 5A). This was in contrast to the wild-type, in which no fatality was observed in the initial 24 h, with the majority (73%) surviving long-term (FIG. 5A). Moreover, the complete mortality in the Kir6.1-KO (10/10 at 18 h) following 15 μg/g LPS greatly exceeded that observed in mice lacking non-vascular, including myocardial, K_{ATP} channels through genetic ablation of the non-vascular Kir6.1 homologue Kir6.2 (3/17 at 24 h and 6/17 long-term; P<0.0001). In the absence of Kir6.1, the high susceptibility to endotoxin-mediated stress was confirmed over a range of LPS concentrations (FIG. 5B; P=0.009), with a readily demonstrable early death following LPS (FIG. 5C) in the Kir6.1-KO (n=39) compared to the wild-type (n=61; P=0.0055). The LD50 to LPS at 12 h was 27-fold higher for Kir6.1-KO (6.7 μg/g) than wild-type (185.4 μg/g; P<0.0001). Death in the Kir6.1-KO (n=4) was preceded by progressive decline in cardiac activity captured on telemetry (FIG. 5D). This was in contrast to the wild-type (n=3), in which cardiac activity was preserved (FIG. 5D).

Lack of Kir6.1 Compromises Cardiac Adaptive Performance in Endotoxic Shock. The development of cardiovascular impairment is a critical determinant of mortality in severe sepsis and septic shock, with optimal cardiac function increasing survival (Natanson et al., 1989, supra; Suffredini et al., 1989, supra). In the experiments reported herein, wild-type mice had normal electrocardiograms both prior to and following LPS challenge. Electrocardiograms of the Kir6.1-KO at baseline showed intermittent transient abnormalities due to lack of coronary K_{ATP} channel activity (Miki et al., 2002, supra), however, demonstrated severe and persistent ST segment change following LPS administration (FIG. 5F). Severe and persistent ST segment change is an electrophysiological marker for the development of global myocardial ischemic injury.

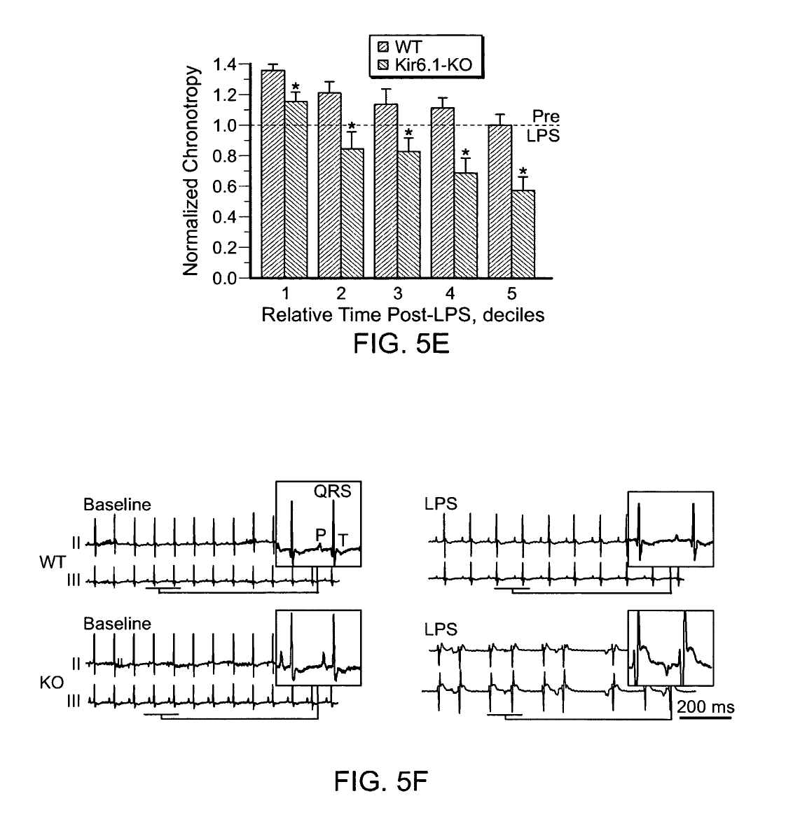

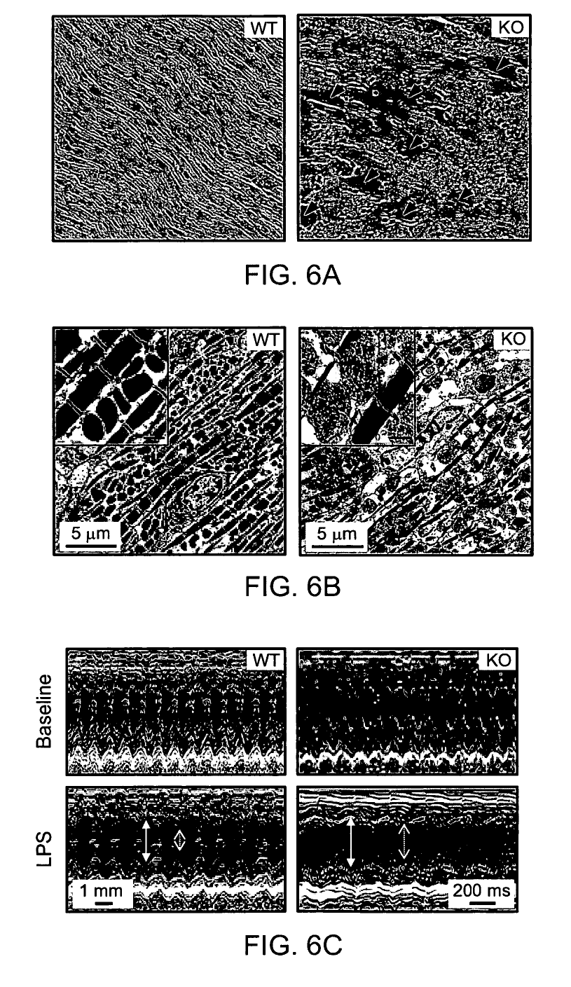

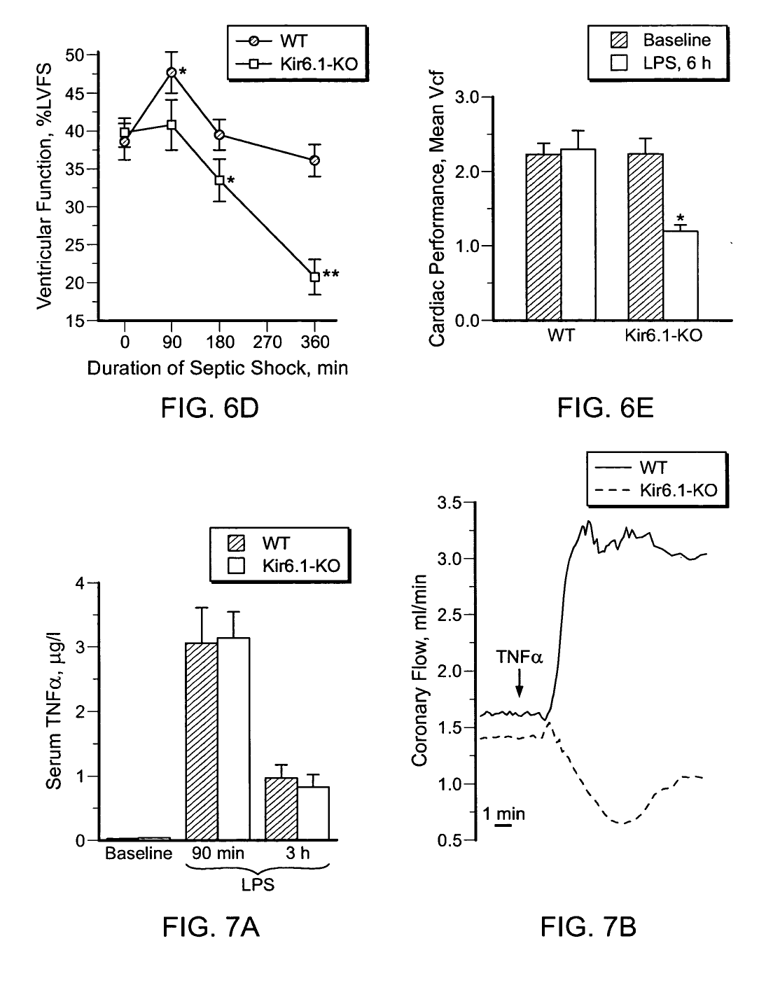

At autopsy, hearts from Kir6.1-KO mice displayed early features of myocyte coagulation necrosis with cytoplasmic hypereosinophilia within 4-6 h of LPS administration (n=8), pathologic findings that were absent in endotoxic wild-type (n=8; FIG. 6A) or saline treated Kir6.1-KO control hearts (n=6). Distinct from the normal architecture of wild-type hearts, the ultrastructure of cardiomyocytes in Kir6.1-KO hearts demonstrated disarray (FIG. 6B) with swollen, amorphous deposit-laden mitochondria (FIG. 6B inset). Such pathologic findings in the LPS-challenged Kir6.1-KO, indicative of ischemic cardiomyocyte injury, translated into contractile dysfunction, which was documented non-invasively by serial echocardiography.

While both the wild-type (n=10) and Kir6.1-KO (n=10) were normal prior to LPS administration (FIG. 6C, D and E), only the wild-type augmented cardiac performance early in response to LPS (% fractional shortening increased from 39±2 to 49±4; P<0.05) and maintained normal contractility through 3 and 6 h (FIG. 6C, D and E). In contrast, Kir6.1-KO mice displayed no early positive inotropy (% fractional shortening of 40±2 versus 42±4, P=0.64; FIGS. 6C and D) and progressed to left ventricular failure documented by deterioration of both left ventricular fractional shortening (FIG. 6D) and circumferential shortening velocity (FIG. 6 E). Cardiac dysfunction in Kir6.1-KO hearts was mediated by a marked elevation in end-systolic left ventricular diameter (54±13% increase from baseline, P<0.02), whereas no left ventricular dilation was noted in the wild-type (−3.3±5%, P=0.6; FIG. 6C). Furthermore, Kir6.1-KO was deprived of tachycardia that accompanied the augmented cardiac performance in the endotoxic wild-type (FIG. 5B). Three hours after LPS, wild-type increased heart rates by 16±6% (P<0.05), a response not observed in Kir6.1-KO mice (−1±7%; P=0.92). In fact, in Kir6.1-KO mice at 6 h, heart rates had declined by 33±6% (P<0.02) to 360±19 beats per min while rates remained normal in wild-type at 476±22 beats per min (P=0.62). Thus, in response to the demands imposed by LPS, Kir6.1-KO mice failed to augment cardiac performance and developed marked ST segment elevation preceding and persisting in parallel with the progression to extreme bradycardia, extensive cardiomyocyte injury, and severe contractile dysfunction.

In comparison to the wild-type (n=4) that maintained body temperature at 37.3±0.2° C. and 35.3±1.0° C. before and at 6 h post-LPS(P=0.2), respectively, the cardiac-impaired endotoxic Kir6.1-KO (n=3) developed hypothermia, dropping from 36.5±0.5° C. to 29.3±1.0° C. (P<0.02), respectively. These systemic perturbations in the endotoxic Kir6.1-KO (n=5) were associated with metabolic acidosis characterized by lower serum pH (7.14±0.02 versus 7.22±0.05; P<0.05) and higher serum lactate (4.0±1.6 mM versus 1.8±0.2 mM; P<0.05) not seen in wild-type (n=6). Non-cardiovascular parameters including the typical disturbance of hypoglycemia were comparable between wild-type (serum glucose from 166±15 to 52±14 mg/dl; P<0.001) and Kir6.1-KO (from 152±4 to 44±6 mg/dl; P<0.001), as was blood oxygen concentration (68.6±9 mm Hg in wild-type and 62.3±13 mm Hg in Kir6.1-KO; P=0.67). These results indicated that vascular K_{ATP} channels contribute to the attainment of the energetically-demanding hyperdynamic cardiac state and maintenance of metabolic stability in endotoxic shock.

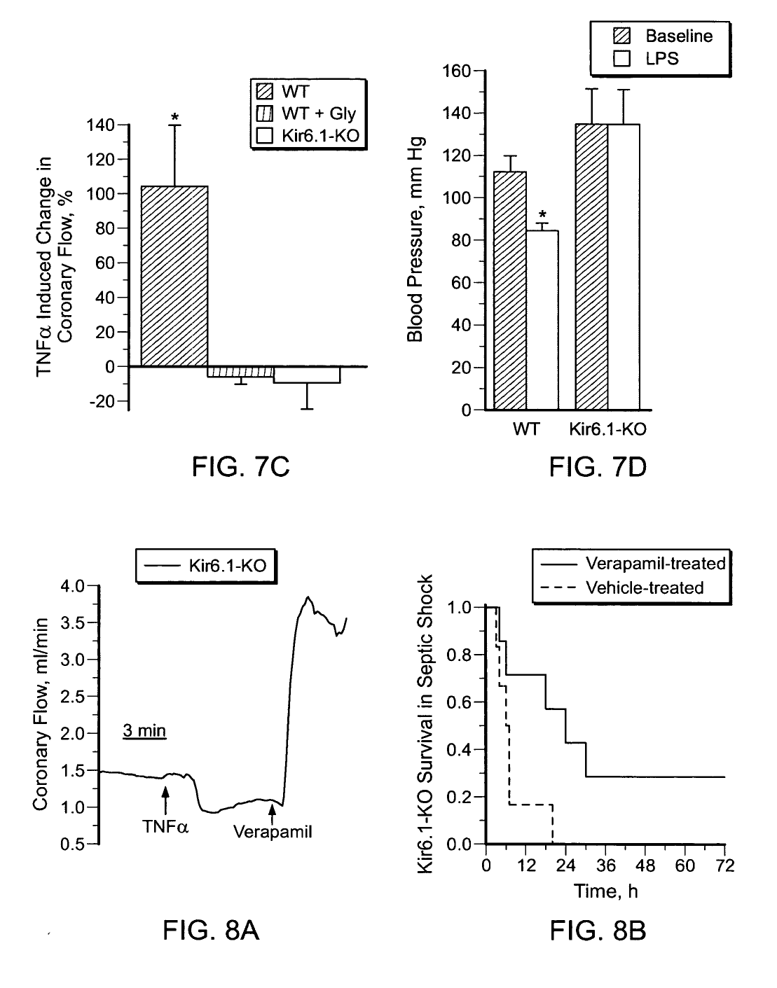

Loss of Cytokine-Induced Vasodilation in Kir6.1-KO Compromises Coronary Flow. The septic response is distinguished by a pro-inflammatory mediator cascade that activates cellular defense mechanisms required to combat infection (Cohen, 2002, Nature, 420:885-91). A critical early common denominator of this response to infectious organisms and their endotoxins is the cytokine TNFα that acts as an early mediator of the vasodilatory septic shock syndrome (Tracey et al., 1986, Science, 234:470-4). Both Kir6.1-KO and wild-type mice had undetectable serum TNFα levels prior to LPS that rose to a similar extent following LPS injection, peaking at 90 min (FIG. 7A). This suggests that the observed early cardiac dysfunction in the endotoxic Kir6.1-KO was independent from the LPS-induced TNFα dynamics, implying a compromise in the host response to an otherwise equivalent cytokine surge.

Because the TNFα-initiated hyperdynamic syndrome of severe sepsis comprises increased cardiac output with vasodilation, the vasodilatory capacity of the coronary bed was evaluated ex vivo in wild-type and Kir6.1-KO. While having similar rates of coronary flow at baseline (P=0.98), wild-type and Kir6.1-KO displayed a differential cytokine-induced dilatory response. Recombinant TNFα, administered at a dose (4 μg/l) comparable to the peak serum level observed in vivo (FIG. 7A), induced maximal coronary vasodilation in wild-type hearts (FIGS. 7B and C). This local TNFα-induced increase in flow, however, was not observed in Kir6.1-KO (at 4 μg/l or indeed at higher doses of 8 or 20 μg/l TNFα) or in wild-type hearts pre-treated with the K_{ATP} channel inhibitor, the sulfonylurea, glyburide (FIGS. 7B and C). These results indicate that unimpeded channel activity is required to secure vascular smooth muscle tone regulation under a central early mediator of endotoxic and septic shock. Furthermore, mirroring the lack of response at the coronary level, Kir6.1-KO did not systemically vasodilate (mean arterial pressure from 95±8 mm Hg before to 103±15 mm Hg after LPS, P=0.6), unlike the wild-type mice that manifested pronounced systemic vasodilation indicated by a 29±4% fall in mean arterial pressure (85±6 mm Hg to 58±3 mm Hg, P<0.0001; FIG. 7D). Thus, vascular K_{ATP} channels mediate the adaptive vasodilatory response to endotoxic shock.

Rescue of Adaptive Vascular Response Offsets LPS-induced Mortality in Kir6.1-KO. Absence of K_{ATP} channel activity predisposes the heart to calcium-dependent smooth muscle constriction. Rescue of the vasodilatory response in Kir6.1-KO was achieved herein by bypassing the defective capacity of vessels to secure flow (FIG. 8A). Application of the calcium channel antagonist, verapamil, to Kir6.1-KO hearts challenged with TNFα reinstated coronary vasodilation (FIG. 8A) to a level comparable with the maximum flow response of the wild-type (FIG. 7B). In fact, calcium channel inhibition negated the disproportionate LPS-induced mortality in the Kir6.1-KO (P<0.05, FIG. 8B) resulting in a significant increase in median survival time from 7 to 24 h. Restoration of the vascular response to endotoxin-mediated stress thereby secures adequate organ perfusion, translating into the prevention of premature and exaggerated mortality in endotoxic shock.

### Part III. Kir6.2 Knockout Mice and Hypertension

Example 1—Materials & Methods

K_{ATP} Channel Knockout and Experimental Hypertension. K_{ATP} channel-deficient mice (Kir6.2-KO) were generated by targeted disruption of the KCNJ11 gene (Miki et al., 1998, PNAS USA, 95:10402-6) which encodes the pore-forming Kir6.2 channel subunit of myocardial but not renal or vascular K_{ATP} channels, and backcrossed for 5 generations to a C57BL/6 background. With approval of the Mayo Foundation Institutional Animal Care and Use Committee, Kir6.2-KO or 8- to 12-week old, age-matched, C57BL/6 male wild-type mice had left nephrectomy through a retroperitoneal flank excision under isoflurane-anesthesia to reduce clearance and mineralocorticoid-hypertension was induced for 21 days by subcutaneous implantation of a 50 mg 21-day-release deoxycorticosterone-acetate pellet (Innovative Research of America) and drinking water supplementation with 1% NaCl/0.2% KCl. Control wild-type or Kir6.2-KO mice underwent nephrectomy alone. Water or salt water intake was measured weekly. All mice were given standard rodent chow, had similar awake non-fasting blood glucose levels measured by tail sampling (Table 2; OneTouch Ultra, Lifescan), were housed individually with a 12 h day/night cycle, and were observed daily until termination of the study at day 21. Following one-week of acclimatization, blood pressure was measured by automated tail-cuff recording (Columbus Instruments) in awake restrained wild-type and Kir6.2-KO mice, 2 weeks post-nephrectomy with or without mineralocorticoid/salt loading. Systolic blood pressure was digitally derived from ten sequential recordings.

TABLE 2
Mixed-meal Glucose Testing In Vivo

| | WT control | WT HTN | KO control | KO HTN |
|---|---|---|---|---|
| N = | 8 | 10 | 8 | 10 |
| Blood glucose | 159 ± 6 | 141 ± 7 | 139 ± 4 | 135 ± 7 |
| (mg/dL) | | | | |

WT, wild-type;
HTN, hypertensive;
KO, Kir6.2 knockout.
P = 0.3 between groups

Gross Pathology and Tissue Fibrosis. At 21 days, whole heart, left ventricle including septum, kidney and lungs were removed, rinsed, blotted dry, and weighed ex vivo. Lung samples were dried at 65° C. for 48 h and reweighed with pulmonary congestion assessed by comparing wet to dry lung weight ratios. Interstitial fibrosis was quantified by computer analysis (MetaMorph, Visitron Universal Imaging) of 0.5-μm thick, paraffin-embedded, Masson's trichrome-stained sections. Kidney or left ventricular collagen content was determined by assaying hydroxyproline content after overnight hydrolysis by 12 N HCl with samples run in duplicate against standard hydroxyproline (Sigma). All quantification was performed blinded to the sample origin.

Treadmill Exertion. To assess the impact of hypertension on exercise capacity, a comparison of performance was made on a graded treadmill exercise test at day 21 between hypertensive and control wild-type and Kir6.2-KO mice. Workload (J) was calculated as the sum of kinetic (Ek=mv²/2) and potential (Ep=mgvt[sin θ]) energy, where m is animal mass, v is treadmill velocity, g is acceleration due to gravity, t is elapsed time at a protocol level, and θ is the angle of incline.

Electrocardiography and Telemetry. To continuously monitor heart rate and electrocardiogram in the conscious state, telemetry devices (Data Sciences International) were implanted in the peritoneum and leads were tunneled subcutaneously in a lead II configuration under isoflurane anesthesia in wild-type and Kir6.2-KO mice. Following a 2-week recovery from surgery, serial signals were acquired at 2 kHz before and during mineralocorticoid/salt challenge. Surface electrocardiogram recordings were obtained under light anesthesia (1.25% isoflurane) via limb lead electrodes. The QT interval was defined as the time from start of the Q wave to the end of the T wave (time when repolarization returned to the isoelectric point) of the electrogram. Corrected for heart rate, the QT interval (QTc) was calculated as QT_{c}=QT/\sqrt{RR/100}, where RR (in ms) is the interval between two consecutive R waves (Zingman et al., 2002, supra).

Cardiac Ultrasound. Echocardiography (c256 and 15L8, Acuson) was performed in lightly sedated (1.25% isoflurane) mice at the end of the 21-day protocol. Images were digitally acquired and stored for off-line blinded analysis. Echocardiographic measurements of left ventricular dimensions were recorded at end-diastole (EDD) and end-systole (ESD) from 3 consecutive cardiac cycles using the leading edge method. LV fractional shortening (% FS) was calculated as: % FS=[(EDD−ESD)/EDD]100. Ejection time (Et) was determined from the actual pulsed-wave Doppler tracings on the parasternal long-axis view of trans-aortic flow by measuring the interval from the beginning of the acceleration to the end of the deceleration. The myocardial velocity of left ventricular circumferential shortening (Vcf expressed in circumferences per s) was calculated as: Vcf=[(EDD−ESD)/EDD]/Et. Stroke volume was determined by the sum of aortic root cross sectional area and the velocity time integral, taken from peak trans-aortic Doppler tracings. The product of stroke volume and heart rate, expressed as ml/min, was used to calculate cardiac output.

Left Ventricular Catheterization. Invasive left ventricular pressure recordings were measured in vivo by a 1.4-Fr micropressure catheter (SPR-671, Millar Instruments) following carotid arterial cannulation and advancement across the aortic valve under 2,2,2 tri-bromoethanol (375 μg/kg i.p.; Sigma) anesthesia, before and after dobutamine (15 mg/kg i.p.) challenge. Left ventricular developed pressure was defined as the absolute difference between the maximum and minimum left ventricular pressure (mm Hg) and the relaxation time as the time from maximal rate of pressure decay to minimum left ventricular pressure (ms).

Isolated Cardiomyocytes, Immunohistochemistry and Calcium Imaging. The aorta was cannulated in situ, and the heart was rapidly excised, retrogradely perfused, and cardiomyocytes were enzymatically dissociated (Hodgson et al., 2003, supra). Surface area measurements of rod-shaped striated calcium-tolerant ventricular cardiomyocytes were performed by digital planimetry (MetaMorph; Visitron Universal Imaging). For immunohistochemistry, formalin-fixed, paraffin embedded left ventricular sections were deparaffinized with xylene and rehydrated in serial alcohol washes. To optimize antigen retrieval, sections were incubated in 0.5 M NH₄Cl with 0.25% Triton X-100 for 30 min, and then for an additional 30 min in 1 mM EDTA in a pressure cooker. Left ventricular sections or 3% paraformaldehyde-fixed isolated cardiomyocytes were probed with primary antibodies applied at 4° C. overnight to the cardiac sarcomeric protein α-actinin (mouse polyclonal, 1:500; Sigma), the K_{ATP} channel pore Kir6.2 (goat polyclonal, 1:300; Santa Cruz), and the cardiac transcription factors—MEF2C (rabbit polyclonal, 1:300; Cell Signaling Technologies) and NF-ATc4 (rabbit polyclonal, 1:300; Santa Cruz).

Accordingly, Alexa 568-labeled anti-mouse (1:200), Alexa 488-labeled anti-goat (1:200) and Alexa 488-labeled anti-rabbit (1:200) secondary antibodies (Molecular Probes) were applied for 60 min, along with nuclear counter-staining achieved by a 3-min application of 300 nM 4′,6′-diamidino-2-phenylindole hydrochloride (DAPI; Molecular Probes). Images were acquired by laser confocal microscopy (Zeiss LSM 510 Axiovert) as described (Behfar et al., 2002, FASEB J, 16:1558-66). For calcium measurements, freshly isolated rod-shaped striated ventricular cardiomyocytes loaded with the calcium-fluorescent probe Fluo-4-acetoxymethyl ester (2 μM; Molecular Probes) were scanned using the 488 nm line of an argon/krypton laser in an oxygenated chamber at 37±1° C. Two-dimensional confocal images (Zeiss LSM 510 Axiovert) of cells from matched hypertensive wild-type and Kir6.2-KO hearts were deconvoluted, and analyzed using Metamorph, normalized to the degree of background fluorescence (Zingman et al., 2002).

Nucleotide Content. Nucleotide levels were determined in 0.6 M perchloric acid/1 mM EDTA extracts from liquid N₂ freeze-clamped hearts (Zingman et al., 2002, supra). Extracts were neutralized with 2 M K₂HCO₃, and nucleotides, eluted with a linear gradient of triethyl ammonium bicarbonate buffer, were profiled by high-performance liquid chromatography (HP 1100, Hewlett-Packard) with a MonoQ HR5/5 column (Amersham Pharmacia; Dzeja et al., 2002, PNAS USA, 99, 10156-61). As an integrated indicator of the overall myocardial bioenergetic state, the DP/TP nucleotide index was defined as the product of the ratios of nucleotide diphosphates (DP) to their respective triphosphates (TP), i.e. [ADP/ATP]·[GDP/GTP]·[UDP/UTP].

Calcineurin Activity. Calcineurin enzyme activity was obtained by quantification of phosphatase activity in left ventricular desalted cytosolic extracts, normalized to protein content, as determined by assay (Bio-rad). Calcineurin phosphatase activity was measured in duplicate using the R11 phosphopeptide as substrate in okadaic acid by the average difference in free-phosphate released in the presence and absence of EGTA as detected by Malachite green assay at OD₆₂₀ (CN Biosciences Corporation).

Nuclear extracts and electrophoretic mobility shift assay (EMSA). Nuclear extracts were prepared from left ventricular tissue in the presence of complete mini protease inhibitor (Roche), phosphatase inhibitors, 10 nM staurosporine, 1 mM dithiothreitol, and 1 mM phenylmethylsulfonyl fluoride on ice or at 4° C. Tissue was resuspended in 10 ml/g of hypotonic buffer (10 mM Tris pH 7.5; 1 mM MgCl₂; 10 mM NaCl; 5 mM CaCl₂) and dissociated in a glass dounce homogenizer with 15 strokes of a loose fitting pestle. Cellular debris was removed by a 30 s centrifugation at 2,000 rpm in a microcentrifuge. The nuclei were recovered from the supernatant by centrifugation at 1,700 g for 10 min. Nuclei were washed by resuspension in 1 ml of nuclear resuspension buffer (10 mM Tris pH 7.5; 250 mM sucrose; 1 mM MgCl₂; 0.1 mM EDTA) and collected by centrifugation as above. Nuclei were resuspended in one packed nuclear volume of low salt buffer (20 mM HEPES pH 7.9 at 4° C.; 25% glycerol; 1.5 mM MgCl₂; 20 mM KCl; 0.2 mM EDTA). Then, one packed nuclear volume of high salt buffer (20 mM HEPES pH 7.9 at 4° C.; 25% glycerol; 1.5 mM MgCl₂; 800 mM KCl; 0.2 mM EDTA) was added. Samples were incubated on ice for 30 min with intermittent mixing. Salt concentration was brought to 833 mM by addition of 2.2 μl of 3 M KCl per 10 μl of sample volume and samples were incubated on ice for 30 min. The nuclear extract was diluted with 1.5 volumes of nuclear diluent (25 mM HEPES pH 7.9 at 4° C.; 0.1 mM EDTA) and clarified by centrifugation at 16,000 g for 15 min. The supernatant (nuclear extract) was saved at −20° C.

EMSAs were performed with 6 μg of nuclear extract and 20 fmoles of end-labeled probe (BNP-927 sequence, Molkentin et al., 1998, Cell, 93:215-28) in Binding Buffer (10 mM HEPES pH 7.9 at 4° C.; 50 mM NaCl; 1 mM MgCl₂; 1 mM CaCl₂; 10% glycerol; 100 μg/ml bovine serum albumin; 0.167 mg/ml polydl-dC). Extract was preincubated 15 min at room temp with Binding Buffer, then probe was added and incubation continued for 30 min at room temp. For competition reactions, a 50-fold molar excess (i.e., 1 pmol) of unlabeled wild-type or mutated NFAT consensus sequence (Santa Cruz Biotechnology) was added after the 15 min preincubation for 15 min at room temp prior to probe addition. For immunodepletion experiments, 100 μg of nuclear extract was incubated overnight on ice with 1 μg of NFATc4 (NFAT3) antibody (Santa Cruz Biotechnology) in a 100 μl volume containing 1 mg/ml of bovine serum albumin and a protease inhibitor cocktail (mini complete, Roche). Protein A support resin was added (35 μl of a 50% slurry, Pierce) and incubation continued on ice for 3 hr. The resin was recovered by centrifugation at 10,000 rpm for 30 sec in a microcentrifuge. The supernatant (i.e., NFAT3-depleted) was transferred and stored at −20° C. The resin was washed 3 times with phosphate buffered saline (PBS) containing 0.1% nonidet P40 (NP-40) and once in PBS. Protein was eluted from resin by incubation on ice for 5 min in 25 μl of Elution Buffer (pH 2.5-3.0) (Seize Protein A Immunoprecipitation kit, Pierce). The resin was separated from the eluate by centrifugation as above and the supernant (i.e., recovered NFAT3) was neutralized by addition of 0.05 volumes of 1M Tris pH 8.9 and stored at −20° C. Seven μl of the eluate was used in EMSA to determine the recovery of NFAT3 DNA binding activity.

Immunoblotting. Forty-five μg of nuclear extract was resolved on denaturing 10% polyacrylamide gels containing 0.05% bisacrylamide. Proteins were transferred to Hybond C nitrocellulose (Amersham) in 25 mM Tris base; 192 mM glycine; 0.025% SDS; 10% methanol for 2 hrs at 395 mA at 4° C. using a submersible plate electrode system (Bio-Rad). Following transfer, membranes were allowed to air-dry overnight. Membranes were incubated for 1 hr in Blocking Buffer (3% nonfat dry milk in TBST [50 mM Tris pH 7.5; 150 mM NaCl; 0.05% Tween 20]), washed 3×5 min in TBST, and placed in 2 μg/ml of NFATc4 antibody (Santa Cruz Biotechnology) in 0.5% nonfat dry milk in TBS (50 mM Tris pH 7.5; 150 mM NaCl) overnight at 4° C. Membranes were washed 1×10 min and 2×5 min in TBST and placed in anti-rabbit IgG horse radish peroxidase conjugate diluted 1:24,000 in TBS for 1 hr at room temp. Membranes were washed 3×5 min in TBST and 1×5 min in TBS. Membranes were incubated in Super Signal West Pico Substrate (Pierce) and the signal was captured on the AutoChemi System (UVP, Inc.). Signals were quantified using LabWorks software (UVP, Inc.)

Pharmacologic Intervention in vivo. Murine subgroups were treated with either 25 mg/kg i.p. of the calcineurin inhibitor cyclosporine A (Sandimmune, Novartis) every 12 h or with 250 μg of the L-type calcium channel antagonist verapamil (Sigma) orally daily, starting the day of nephrectomy with or without deoxycorticosterone-acetate/salt loading.

Statistical Analysis. Comparisons between groups were performed by log-rank, analysis of variance, Student's t tests, or non-parametric tests as appropriate (JMP; SAS). Data are presented as mean±SEM; n refers to sample size. P<0.05 was predetermined.

Example 2—Results

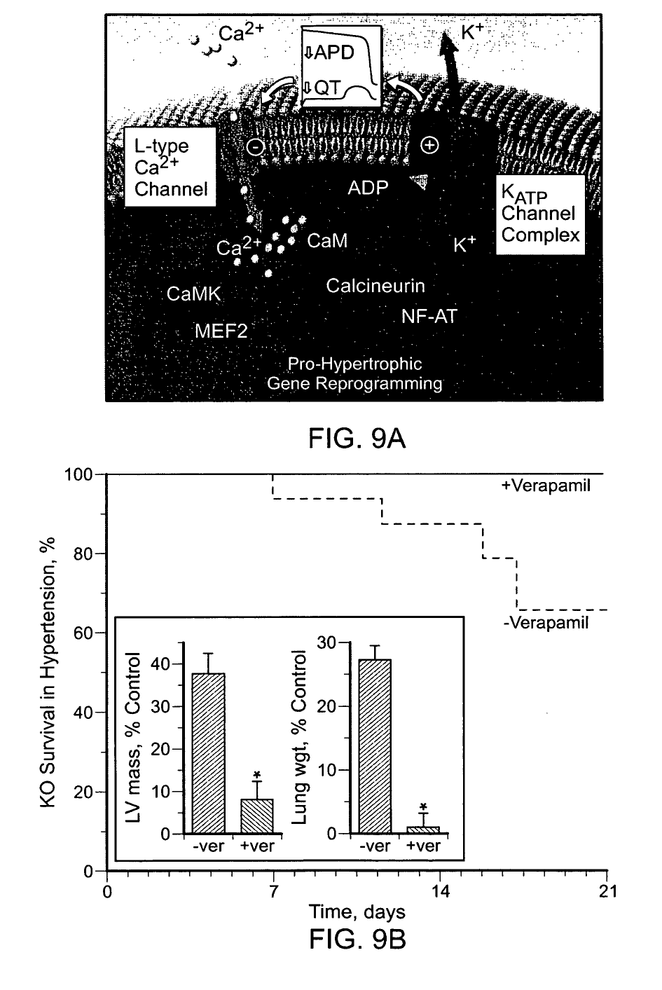

Severe Survival Disadvantage in K_{ATP} Channel Knockout with Hypertension. Hypertension was imposed upon C57BL/6 wild-type and age-matched K_{ATP} channel deficient mice (Kir6.2-KO) generated by targeted disruption of the KCNJ11 gene which encodes the pore-forming Kir6.2 channel subunit (Miki et al., 1998, supra). Chronic 21-day experimental hyperaldosteronism was replicated by incorporating, following uni-nephrectomy, mineralocorticoid challenge through a sustained subcutaneous deoxycorticosterone acetate release combined with oral salt loading. This hemodynamic stress regimen produced a significant and comparable level of systemic hypertension in both wild-type and Kir6.2-KO mice (FIG. 9A). Despite equivalent loading conditions, as further indicated by similar natriuresis-induced increases in fluid intake (FIG. 9B), renal hypertrophy (FIG. 9C) and kidney collagen deposition (FIG. 9D), hypertensive mice displayed a differential exertional response depending on the presence or absence of functional K_{ATP} channels. Unlike the wild-type, genetic deletion of K_{ATP} channels produced discernible impairment in exercise stress tolerance, an integrative indicator of physical endurance (FIG. 9E). Furthermore, in comparison to the wild-type, the challenge of systemic hypertension halved the survival rate in the Kir6.2-KO cohort, with significant mortality occurring between day 7 and the end of the 3-week protocol (FIG. 9F). In contrast, over 95% of wild-type exposed to the same hypertensive regimen survived, as did all wild-type (n=25) or Kir6.2-KO (n=23) treated with nephrectomy alone, which in the absence of mineralocorticoid/salt loading, did not induce hypertension (FIG. 9A). Thus, despite exposure to equivalent chronic hemodynamic stress, lack of Kir6.2-containing K_{ATP} channels translates into an impaired tolerated workload and an overall marked survival disadvantage. This suggests a defective capacity for vital stress adaptation in a K_{ATP} channel-compromised organism in the setting of hypertensive disease.

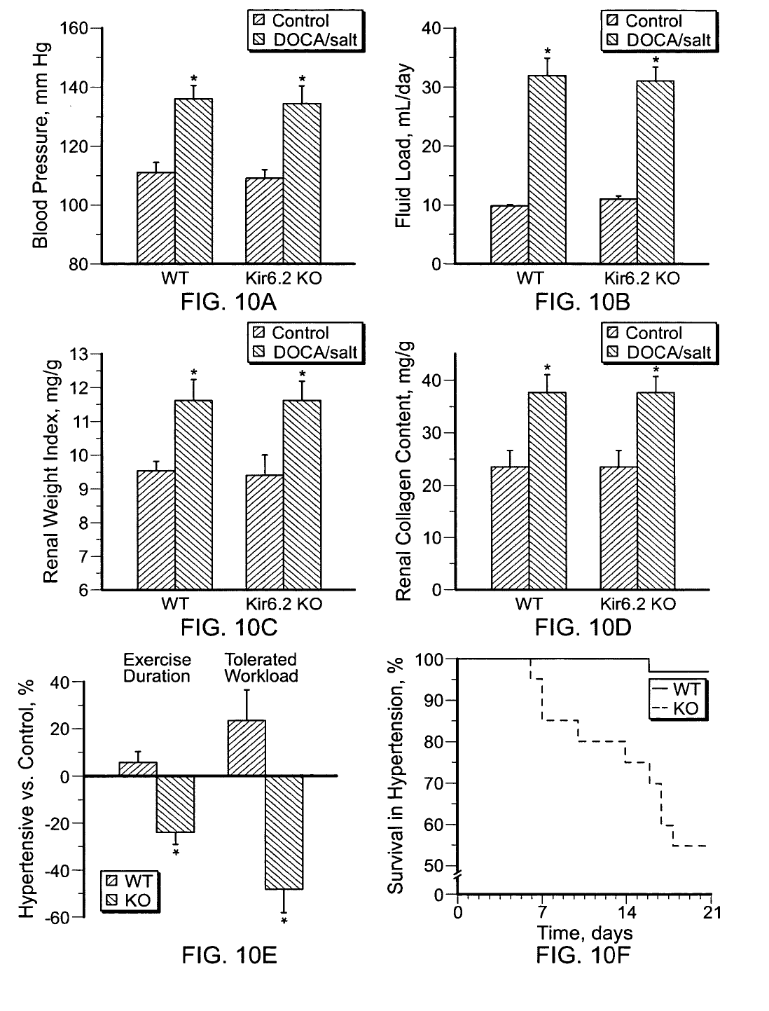

Congestive Heart Failure in Hypertensive K_{ATP} Channel Knockout. Fatal outcome in the hypertensive Kir6.2-KO mice was preceded by a progressive decline in cardiac activity over the days before death (FIG. 10A) with development of atrio-ventricular conduction delay on electrocardiography (FIG. 10A, inset), consistent with the phenotype of a failing heart. This was not observed in wild-type mice. Following mineralocorticoid hypertension, even in surviving animals, hearts lacking K_{ATP} channels developed significant impairment in contractile function captured in vivo by non-invasive echocardiography and cardiac catheterization (FIG. 10; Table 3). While having comparable heart rates, multiple indicators of systolic dysfunction were present in Kir6.2-KO but not in wild-type following mineralocorticoid hypertensive challenge (FIG. 10; Table 3). In contrast to non-hypertensive Kir6.2-KO or hypertensive or non-hypertensive wild-type, all of which had similar and normal systolic function (Table 3), hypertensive Kir6.2-KO mice developed marked left ventricular chamber dilation (left ventricular systolic diameter 2.3±0.1 versus 1.5±0.1 mm in hypertensive Kir6.2-KO and wild-type respectively; P=0.0008; FIG. 10B) and reduced trans-aortic flow velocity (FIG. 10C; Table 3) with overall profound systolic contractile impairment (FIG. 10D; Table 3). Left ventricular systolic dysfunction in the hypertensive Kir6.2-KO was associated with pulmonary congestion (FIG. 10E) indicating that the cardiac contractile dysfunction had progressed into a state of systemic congestive heart failure.

TABLE 3
Non-Invasive and Invasive Cardiac Functional
Assessments In Vivo

| | WT | | | |
|---|---|---|---|---|
| | control | WT HTN | KO control | KO HTN |
| Echocardiography | n = 8 | n = 8 | n = 8 | n = 8 |
| Heart rate, bpm | 492 ± 18 | 467 ± 21 | 497 ± 18 | 466 ± 21 |
| Aortic systolic VTI, cm | 0.524 ± 0.02 | 0.539 ± 0.49 | 0.526 ± 0.04 | 0.382 ± 0.03* |
| Fractional shortening, % | 48.3 ± 1.4 | 47.2 ± 2.8 | 49.4 ± 1.3 | 29.6 ± 2.3* |
| Circ. shortening velocity, | 7.76 ± 0.41 | 7.08 ± 0.72 | 8.25 ± 0.51 | 4.56 ± 0.35* |
| circumferences per s | | | | |
| Stroke volume, ul | 104.7 ± 5.5 | 118 ± 6.10 | 104.7 ± 8.9 | 80.5 ± 9.8* |
| Cardiac output, ml/min | 51.2 ± 2.8 | 54.9 ± 4.8 | 52.9 ± 4.9 | 36.2 ± 2.6* |
| Invasive Hemodynamics | n = 6 | n = 8 | n = 6 | n = 8 |
| Heart rate, bpm | 475 ± 46 | 427 ± 27 | 547 ± 33 | 494 ± 56 |
| LV max. pressure, mm Hg | 82.1 ± 1.2 | 89.3 ± 1.3 | 83.4 ± 3.2 | 61.9 ± 5.9* |
| LV end-diastolic pressure, mm | 3 ± 0.4 | 2.7 ± 0.3 | 3.8 ± 0.3 | 9.8 ± 1.5* |
| Hg | | | | |
| Relaxation time, ms | ND | 44.6 ± 6.6 | ND | 71.4 ± 6.5* |

WT, wild-type;
HTN, hypertensive;
KO, Kir6.2 knockout;
VTI, velocity time integral;
LV, left ventricular;
*P < 0.05 when compared to both WT HTN and KO control;
ND, not determined

In contrast to hypertensive wild-type with preserved cardiac function, Kir6.2-KO counterparts demonstrated defective cardiac inotropy and lusitropy with diminished peak left ventricular developed pressure (FIG. 10F-2H; Table 3) and abnormal prolongation in cardiac relaxation (FIG. 10G-2H; Table 3). Under inotropic challenge with dobutamine, hypertensive Kir6.2-KO mice decompensated to fulminent cardiac dysfunction, with rapid elevation of end-diastolic pressure, and deterioration of developed pressure, eventually precipitating complete cardiac failure and death in all (n=8, P<0.001; FIG. 101). In contrast, hypertensive wild-type, displayed a normal contractile response to the same stress challenge with no death (n=8; FIG. 101). Thus, hypertensive K_{ATP} channel-deficient mice are predisposed to develop congestive heart failure with fatal outcome.

Aggravated Hypertrophic Remodeling in Hypertensive K_{ATP} Channel Knockout Hearts. In hypertension the magnitude of left ventricular mass increase is the critical predictor of long-term prognosis and rate of decompensation to heart failure. The pathophysiologic myocyte response to extrinsic stress such as hypertension is an increase in cell size accompanied by extracellular matrix deposition, the extent of which correlates with outcome.

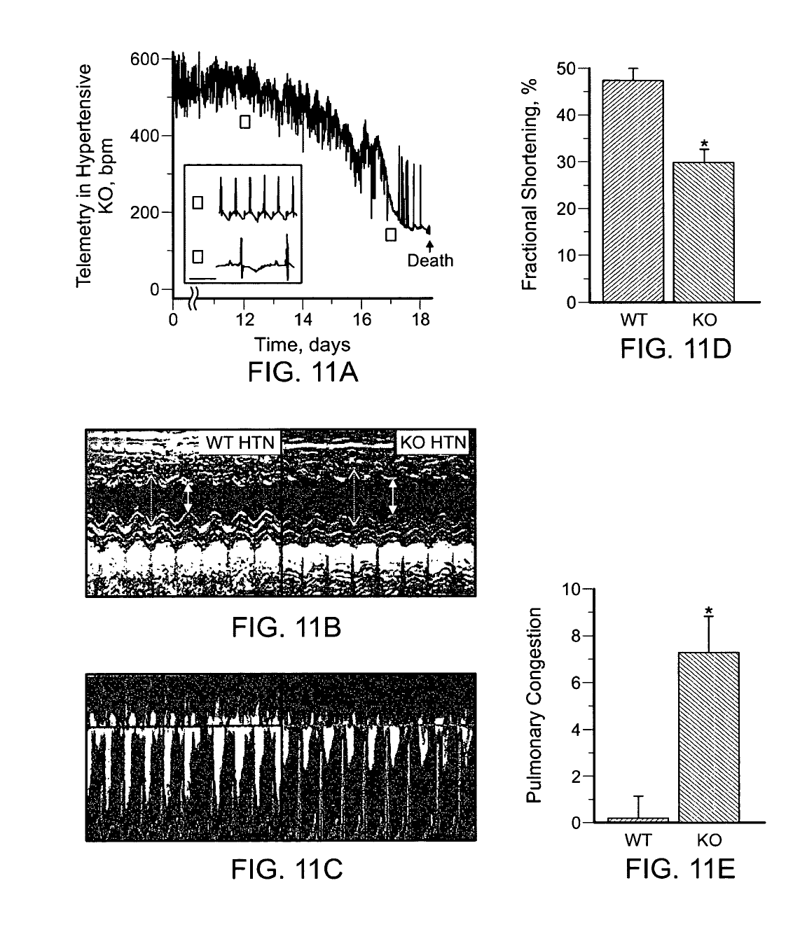

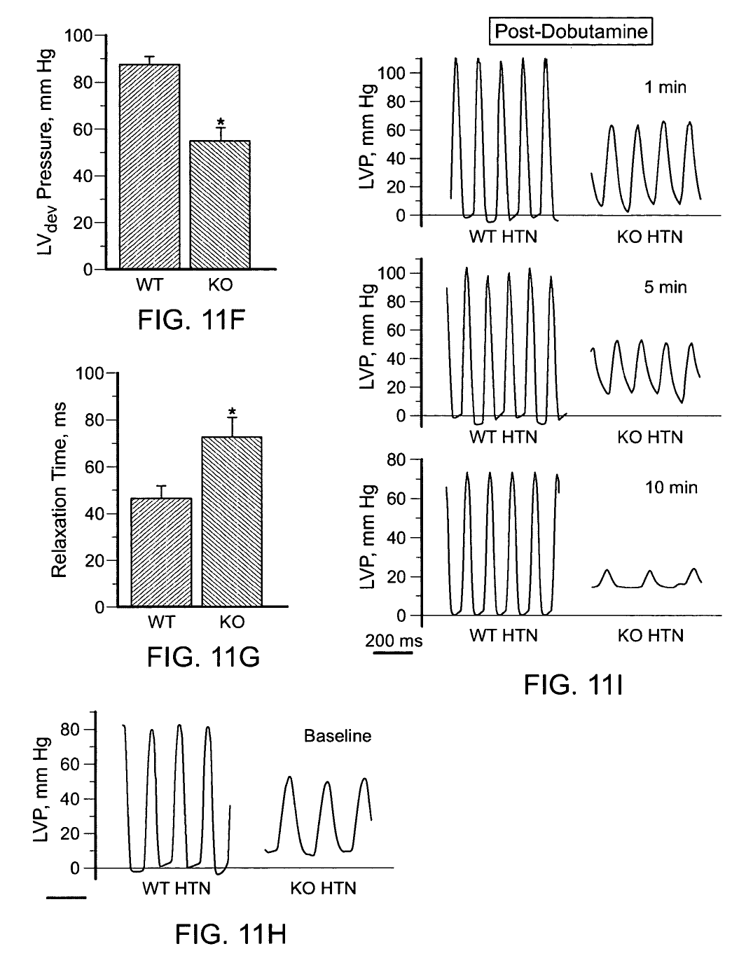

Wild-type hearts responded to hypertension with increases in overall cardiac (FIG. 11A) and more specifically left ventricular size (FIG. 11B) accompanied by interstitial fibrosis (FIG. 11D-E). Despite equivalent hypertensive load, Kir6.2-KO hearts had an excessive hypertrophic response with, on average, more than three times the increase in left ventricular mass observed in the wild-type (FIG. 11A-B). This excessive hypertrophy appeared to closely correlate with outcome as the normalized left ventricular mass from the Kir6.2-KO that died (5.6±4.2 mg/g, n=8) was significantly higher than from those that survived within the 21 days of imposed hypertension (5.02±0.13 mg/g, n=15; P<0.02). Indeed, the mean surface area of individual cardiomyocytes isolated from hypertensive Kir6.2-KO hearts was also significantly greater than those from hypertensive wild-type hearts (FIG. 11C; P<0.03). Furthermore, the extent of extracellular matrix deposition was significantly higher in Kir6.2-KO hearts compared to wild-type (FIG. 11D-E), as was the left ventricular collagen content with a three-fold higher increase over control seen in the Kir6.2-KO versus wild-type (P<0.001). Thus, underlying contractile dysfunction and exaggerated mortality in the hypertensive KATP channel-deficient mice is an excessive maladaptive cardiac remodeling response.

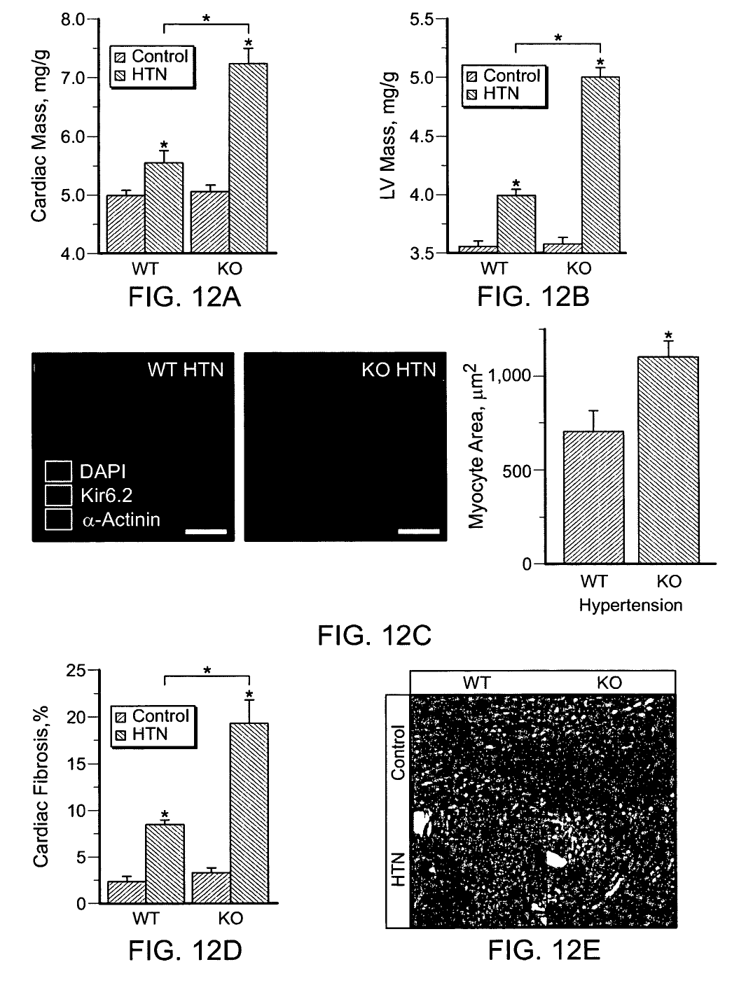

Loss of K_{ATP} Channel Sensing of Hypertrophic Metabolic Distress Precipitates Myocardial Calcium Overload. Consistent with reported metabolic changes in myocardial hypertrophy, hypertensive wild-type or Kir6.2-KO hearts demonstrated a greater than six-fold shift towards increases in nucleotide diphosphate over triphosphate concentrations (FIG. 12A; P<0.005). While such cytosolic energetic compromise is sensed by the K_{ATP} channel complex and is translated into channel activity in the hypertrophic heart, absence of the channel pore prevents cellular distress signal-decoding resulting in loss of stress-induced action potential shortening.

In hypertension, deletion of Kir6.2 resulted in deficient myocardial repolarization, defined as prolongation of the QTc interval on the electrocardiogram (FIG. 12B; P=0.003). Inappropriately prolonged repolarization predisposes to excessive cytosolic calcium loading, and indeed, in contrast to hearts of hypertensive wild-type (FIG. 12C) or non-hypertensive controls that maintained steady intracellular calcium levels, lack of K_{ATP} channels in hypertension precipitated significant cytosolic calcium overload (FIG. 12C; P<0.001). Thus, absence of metabolism-sensing K_{ATP} channels produces bioenergetic-electrophysiologic uncoupling leading to intracellular calcium overload in hypertensive hypertrophic hearts.

Knockout of K_{ATP} Channels Discharges Uncontrolled Calcium-Calcineurin Dependent Signaling. Calcium influx is critical for proper myocyte function, enabling essential excitation-contraction coupling with each beat. Defective calcium cycling with diastolic calcium overload has been associated with predisposition to the development of heart failure and the activation of intracellular calcium/calmodulin-depenaent hypertrophic signaling. At the crossroads of multiple calcium-dependent signal pathways in the heart is the transcription factor, myocyte enhancer factor 2 (MEF2), which, when activated, translocates to the nucleus where it initiates pro-hypertrophic gene reprogramming.

Unlike the hypertensive wild-type which displayed minimal nuclear translocation of MEF2, hearts lacking Kir6.2-containing K_{ATP} channels exhibited nuclei abnormally loaded with MEF2 (FIG. 12D; P<0.001), an indicator of excessive calcium-dependent remodeling signals in the hypertensive Kir6.2-KO hearts. The key calcium-dependent determinant of pathological cardiac hypertrophy upstream of calcium-dependent transcription factors is the serine/threonine protein phosphatase calcineurin that responds to sustained elevations in intracellular calcium. Upon activation by the binding of calcium/calmodulin, calcineurin dephosphorylates the nuclear factor of activated T cells (NF-AT), facilitating nuclear transport where NF-AT mediates pro-hypertrophic gene activation.

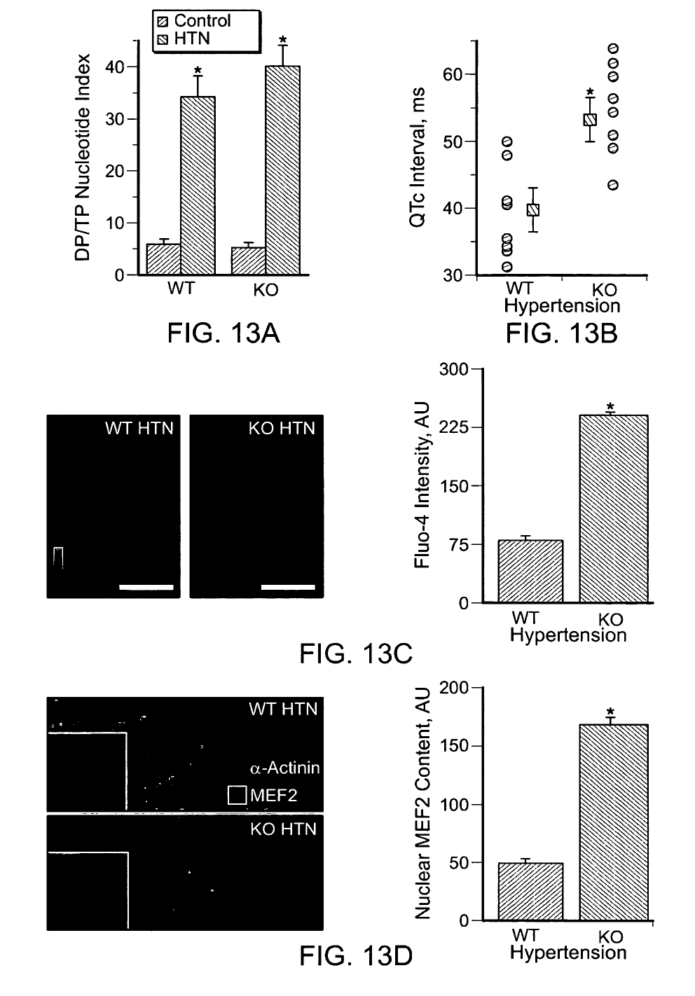

Calcineurin phosphatase activity measured in cytosolic left ventricular extracts was abnormally upregulated in hypertensive Kir6.2-KO compared to the hypertensive wild-type (FIG. 13A; P<0.03). Indeed, in the Kir6.2-KO, the degree of calcineurin activity closely correlated (r=0.82, P<0.02) with the left ventricular mass where the highest level of calcineurin activity was found in hypertensive hearts with the greater degree of cardiomegaly (FIG. 13B). This association between cardiac mass and calcineurin activity was not evident in the wild-type (r=0.22, P=0.3). Furthermore, only hypertensive Kir6.2-KO but not wild-type hearts demonstrated nuclear localization of NF-AT on immunostaining, indicative of marked activation and cytosolic-nuclear shuttling of NF-AT (FIG. 13C). This was confirmed on Western blot analysis of myocardial nuclear extracts in which there was a 7-fold increase in NF-AT from hypertensive Kir6.2-KO over that from control (FIG. 13D, E).

Electrophoretic mobility shift assay in conjunction with immunodepletion demonstrated that the overexpressed NF-AT3 in the nuclei of the hypertensive KO bound the NF-AT consensus sequence in the promoter region of the b-type natriuretic protein gene (FIG. 13F, lanes 1, 4). Specificity was determined by preincubation with a 50-fold molar excess of either the unlabeled wild-type sequence (FIG. 13F, lane 2) or a mutated consensus NF-AT sequence (FIG. 13F, lane 3). Depletion of the nuclear extract of NF-AT3 by immunoprecipitation prevented DNA binding (FIG. 13F, lane 5) with recovery confirmed by adding the immunoprecipitate (i.e., enriched in NF-AT3) to the probe (FIG. 13F, lane 6). Thus, K_{ATP} channel deficiency and the associated inability to respond to signs of metabolic distress under hypertension-induced hypertrophy translated into a reduced ability of the heart to gate calcium-triggered maladaptive genetic reprogramming.

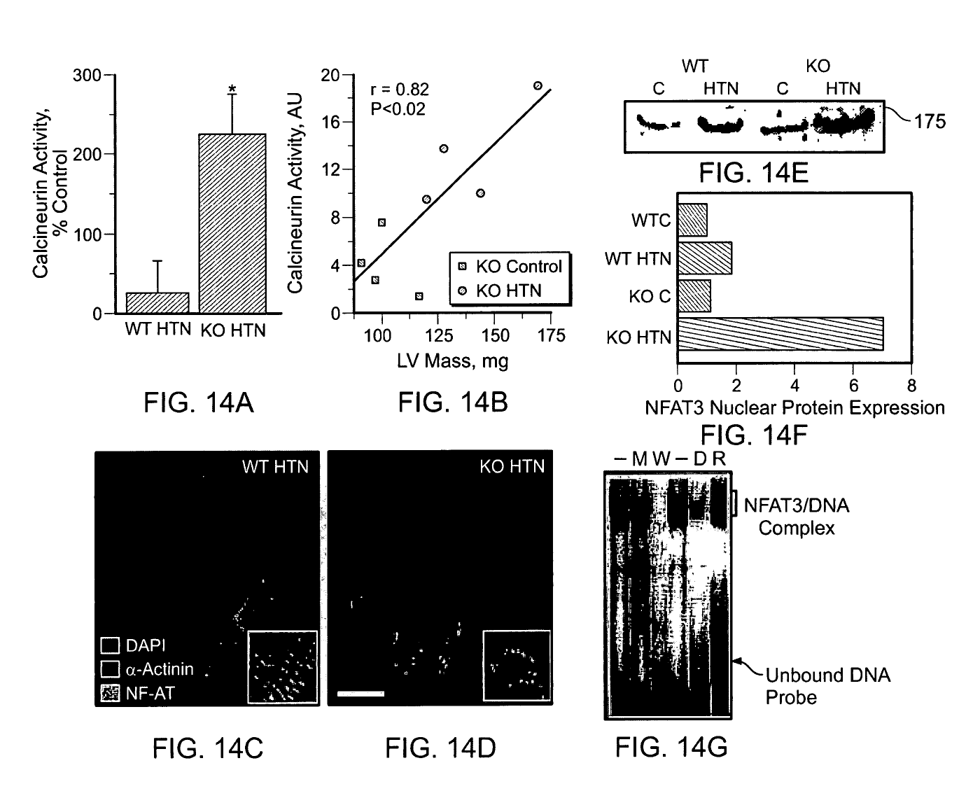

Calcineurin Inhibition Negates Maladaptive Remodeling in Hypertensive K_{ATP} Channel-Deficient Hearts. To examine the significance of such pathologic over-activity of the calcium-calcineurin pathway in hypertensive Kir6.2-KO mice, separate groups of mineralocorticoid/salt-challenged mice were further treated with the calcineurin inhibitor, cyclosporine A. Left ventricular sections taken from hypertensive Kir6.2-KO hearts treated with cyclosporine showed no evidence of nuclear NF-AT staining, implicating prevention of NF-AT activation by pharmacologic calcineurin inhibition (FIG. 14A). Moreover, treatment with the calcineurin inhibitor negated the aggravated cardiac hypertrophy observed in Kir6.2-KO hearts (FIG. 14B). Following cyclosporine therapy, the left ventricular mass of hypertensive Kir6.2-KO hearts was equivalent to that of the hypertensive wild-type, reversing the deficit produced by the lack of K_{ATP} channels (FIG. 14B). Thus, the calcium-calcineurin pathway mediates the aggravated maladaptive cardiac hypertrophy that separates those hearts with and without K_{ATP} channels. This identifies the K_{ATP} channel as a proximal modulator of the calcium-calcineurin pathway under hemodynamic stress.

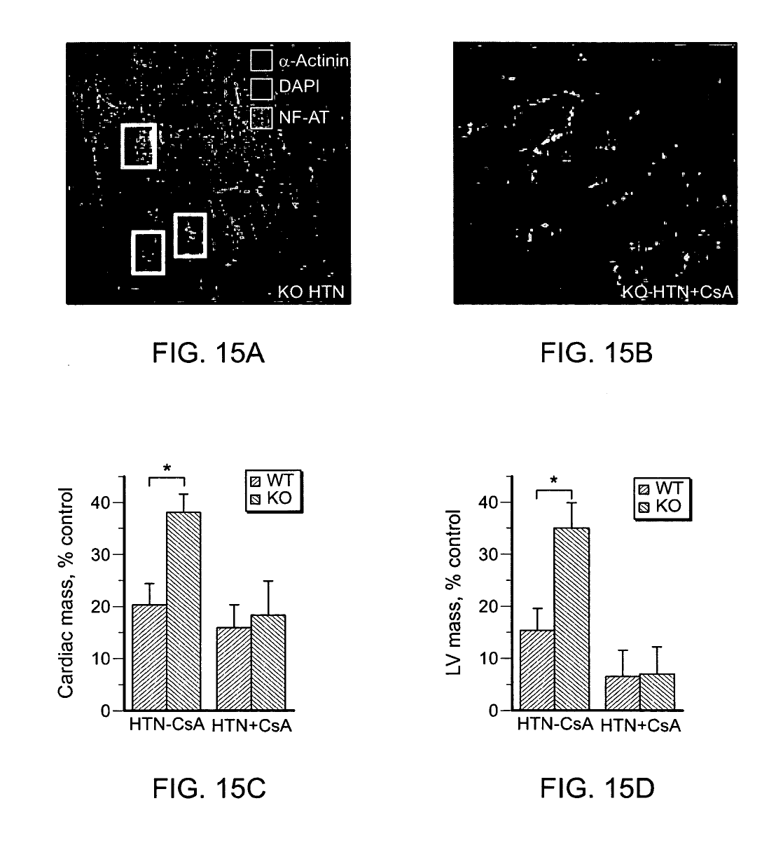

Calcium Channel Antagonism Rescues Hypertensive Kir6.2-KO Phenotype. In the intact heart, K_{ATP} channels respond to derangement of the intracellular nucleotide balance characteristic of a stressed state, as shown here in hypertrophy, with homeostatic shortening of the cardiac action potential securing a feedback mechanism that gates calcium influx through voltage-dependent calcium channels (FIG. 15A). The importance of calcium in the Kir6.2-KO hypertrophic response to mineralocorticoid hypertension was directly probed by treatment of mice with the L-type calcium channel antagonist verapamil that, by directly blocking calcium influx, bypasses the site of K_{ATP} channel action in the calcium handling pathway. While the modest left ventricular hypertrophy in the hypertensive wild-type was not affected by verapamil therapy (P=0.72), verapamil significantly attenuated the increase in left ventricular mass induced by mineralocorticoid/salt loading in the hypertensive Kir6.2-KO (FIG. 15B, inset; P<0.03) at a dose of verapamil that did not affect the degree of systemic hypertension (P=0.52). In fact, verapamil therapy abolished the differential hypertrophic response between the wild-type and Kir6.2-KO (FIG. 15B, inset; P=0.49). Moreover, verapamil treatment averted the syndrome of heart failure including lung congestion in hypertensive Kir6.2-KO (FIG. 15B, inset). Indeed, verapamil-mediated prevention of all mortality in the hypertensive Kir6.2-KO (FIG. 15B) underscored the pathologic role for calcium loading in the stressed K_{ATP} channel-deficient failing heart with rescue of the full phenotype through restoration of a control in calcium influx.

### Part IV. Kir6.2 Knockout Mice and Exercise

Example 1—Materials & Methods

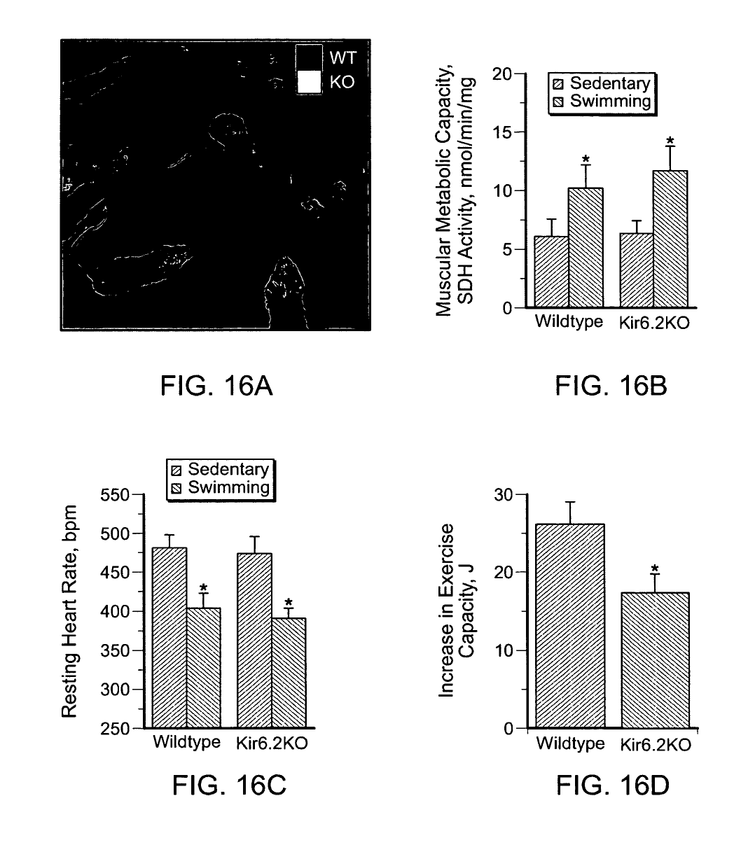

Kir6.2-KO and swimming protocol. K_{ATP} channel deficient mice (Kir6.2-KO) were generated by targeted disruption of the KCNJ11 gene encoding the pore-forming Kir6.2 subunit, and backcrossed for 5 generations to a C57BL/6 background (Miki et al., 1998, supra). Due to the proximity of the mutated KCNJ11 gene with the gene encoding for albino hair color in the SV129 embryonic stem cells used to create the knockout, the Kir6.2-KO mice remain white upon backbreeding into the black C57BL/6 line (FIG. 16A). With approval of the Mayo Foundation Institutional Animal Care and Use Committee, 20-week old male Kir6.2-KO or matched C57BL/6 wild-type mice underwent collective chronic swimming endurance training. Mice swam together in 20 cm deep water (at 33-36° C.) for 90 min twice daily for 28 days. Sedentary control mice were not swum. All mice were given standard rodent chow ad libitum, housed 2-4 per cage with a 12-h day/night cycle and observed daily throughout.

Succinate dehydrogenase. The activity of succinate dehydrogenase (SDH), an index of metabolic capacity, was determined by spectrophotometry in whole tissue homogenates of hamstring muscle groups (Dzeja et al., 2003, Am. J. Physiol., 284:H1048-56). The assay mixture contained 66.7 mM Tris.HCl buffer (pH 8), 6.67 mM KCN, 420 μM phenazine methosulfonate, 86 μM 2,6-dichloroindophenol (DCIP), 1 μM rotenone, and 10 mg/ml skeletal muscle extract. The reaction was initiated by 20 mM succinate, and the rate of DCIP reduction was followed at 600 nm. SDH activity is expressed as μM of reduced DCIP/min/mg of extract protein, with protein content determined by assay (Bio-Rad).

Treadmill. To assess the impact of swimming training on exercise capacity, a comparison of performance was made on a graded treadmill exercise test before and after the swimming protocol. Workload (J) was calculated as the sum of kinetic (Ek=m·v²/2) and potential (Ep=m·g·v·t·sin θ) energy, where m is animal mass, v is treadmill velocity, g is acceleration due to gravity, t is elapsed time at a protocol level, and θ is the angle of incline (Zingman et al., 2002, supra).

Histopathology. Cross sectional area of individual adipocytes was measured on hematoxylin-eosin stained, paraffin embedded, formalin fixed samples of gluteal subcutaneous white or intrascapular brown fat. Typically, 100 cells were measured from each of 5 mice from all groups. Skeletal muscle morphology was examined in hematoxylin-eosin stained hamstring muscle sections by light microscopy. For measurement of cardiac contraction band necrosis, left ventricular hematoxylin-eosin stained sections were examined at low and high magnification from 5-6 mice in each group (Zingman et al., 2002, supra). Cardiac and skeletal muscle ultrastructure was assessed in thin (90-nm) sections cut on an ultramicrotome (Reichert Ultracut E), placed on 200-μm mesh copper grids and stained with lead citrate. Transmitted electron microscopy was performed with a JEOL 1200 EXII electron microscope operating at 60 kV (Zingman et al., 2002, supra). All quantification was performed by an observer blinded to sample origin. For immunohistochemistry, formalin-fixed, paraffin embedded left ventricular sections were deparaffinized with xylene and rehydrated in serial alcohol washes. To optimize antigen retrieval, sections were incubated in 0.5 M NH₄Cl with 0.25% Triton X-100 for 30 min, and then for an additional 30 min in 1 mM EDTA in a pressure cooker. Primary rabbit and mouse polyclonal antibodies were applied, respectively to the cardiac transcription factor myocyte enhancing factor (MEF2C 1:300; Cell signaling technologies) and the cardiac sarcomeric protein (α-actinin 1:500; Sigma) overnight. Accordingly, Alexa 563-labeled antimouse (1:200) and Alexa 488-labeled anti-rabbit (1:200) secondary antibodies (Molecular Probes) were applied for 30 min, along with nuclear counter-staining achieved by a 3 min application of 300 nM 4′,6′-diamidino-2-phenylindole hydrochloride (DAPI; Molecular Probes). Images were acquired by laser confocal microscopy (Zeiss LSM 510 Axiovert) as described (Behfar et al., 2002, supra).

Blood sampling. Plasma leptin levels were quantified by enzyme-linked immunosorbent assay (Crystal Chem Inc.). Blood glucose levels were measured in awake mice by tail sampling (OneTouch Ultra, Lifescan) at 8 am (“fed”) and the following day after a 16 h overnight fast (“fasting”). A glucose tolerance test was performed by the intraperitoneal administration of 1.5 mg/g glucose in fasting mice. In the insulin tolerance test, 0.2 U/kg human insulin was administered intraperitoneally in fed mice. All blood sampling was performed after completion of the 28-day swimming/sedentary protocol.

In vivo hemodynamics. Echocardiography with heart rate measurement (c256 and 15L8, Acuson) was performed in lightly sedated (1.25% isoflurane) mice at the end of the 28-day swimming/sedentary protocol. Images were digitally acquired and stored for off-line blinded analysis. Echocardiographic measurements of left ventricular dimensions were recorded at end diastole (EDD) and end systole (ESD) from 3 consecutive cardiac cycles using the leading edge method (Hodgson et al., 2003, supra; Behfar et al., 2002, supra). Left ventricular fractional shortening (% FS) was calculated as: % FS=[(EDD−ESD)/EDD]×100. Stroke volume was determined by the sum of aortic root cross sectional area and the velocity time index integral taken from peak Doppler tracings from flow across the aortic valve. The product of stroke volume and heart rate, expressed as ml/min/10 g body weight, was used to calculate cardiac output.

Real-time quantitative PCR (qPCR). Total RNA was extracted from left ventricles using the RNEasy Mini Kit (Qiagen). A MEF2C primer was used in real-time PCR analysis with forward 5′-AGA TAC CCA CAA CAC ACC ACG CGC C-3′ (SEQ ID NO:1) and reverse 5′-ATC CTT CAG AGA GTC GCA TGC GCT T-3′ (SEQ ID NO:2) sequences. Reverse transcription and qPCR were preformed as described (Behfar et al., 2002, supra).

Statistical analysis. Comparisons within or between groups were performed by analysis of variance, Student's t tests, or non-parametric tests as appropriate (JMP version 5.1; SAS, Carey N C). Survival was determined by Kaplan-Meier analysis and the log-rank test. Data are presented as mean±SEM; n refers to the sample size. P<0.05 was predetermined.

Example 2—Results

Following a 28-day swimming exercise protocol with equal participation of wild-type and Kir6.2-KO mice (FIG. 16A), both groups displayed physiologic changes that are typical indicators of training. All swum mice displayed significantly enhanced skeletal muscle aerobic capacity measured by succinate dehydrogenase activity (FIG. 16B), significantly lower resting heart rates (FIG. 16C), and superior performance on treadmill stress testing (FIG. 16D). Yet, Kir6.2-KO mice manifested only 66% of the improvement in exercise workload seen in wild-type (FIG. 16D; P<0.05).

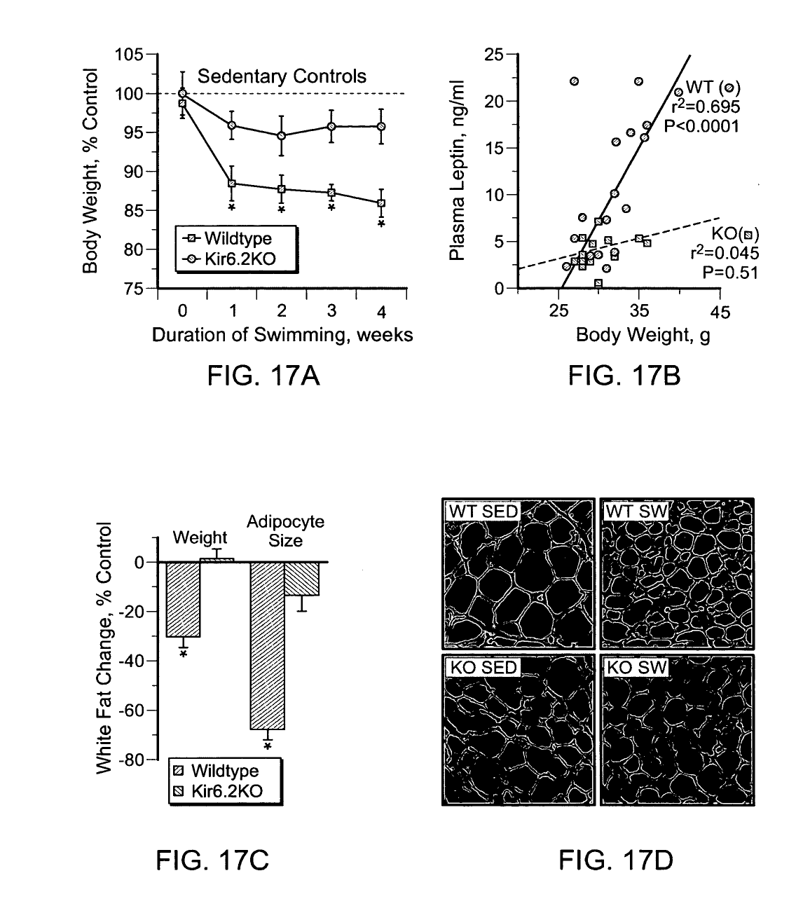

While there was no difference in baseline body weight between groups (P=0.15), in response to swimming, the wild-type became 14.1±1.8% lighter while Kir6.2-KO mice demonstrated no loss in body weight (FIG. 17A). Over this range of body weights, the level of leptin, the metabolic hormone linked to K_{ATP} channel activity, correlated with body mass in the wild-type whereas there was less correlation in mice lacking K_{ATP} channels (FIG. 17B). This differential response to training was mirrored by changes in body fat distribution. Wild-type demonstrated measurable reductions in white fat stores (FIG. 17C), a decline in white adipocyte size (FIG. 17C, D) and a reciprocal increase in brown adipocyte size (37.9±14%, P<0.05). Kir6.2-KO mice had no significant changes in white fat weight (FIG. 17C) or in the size of either white (FIG. 17C, D) or brown adipocytes (−7±13%, P=0.63).

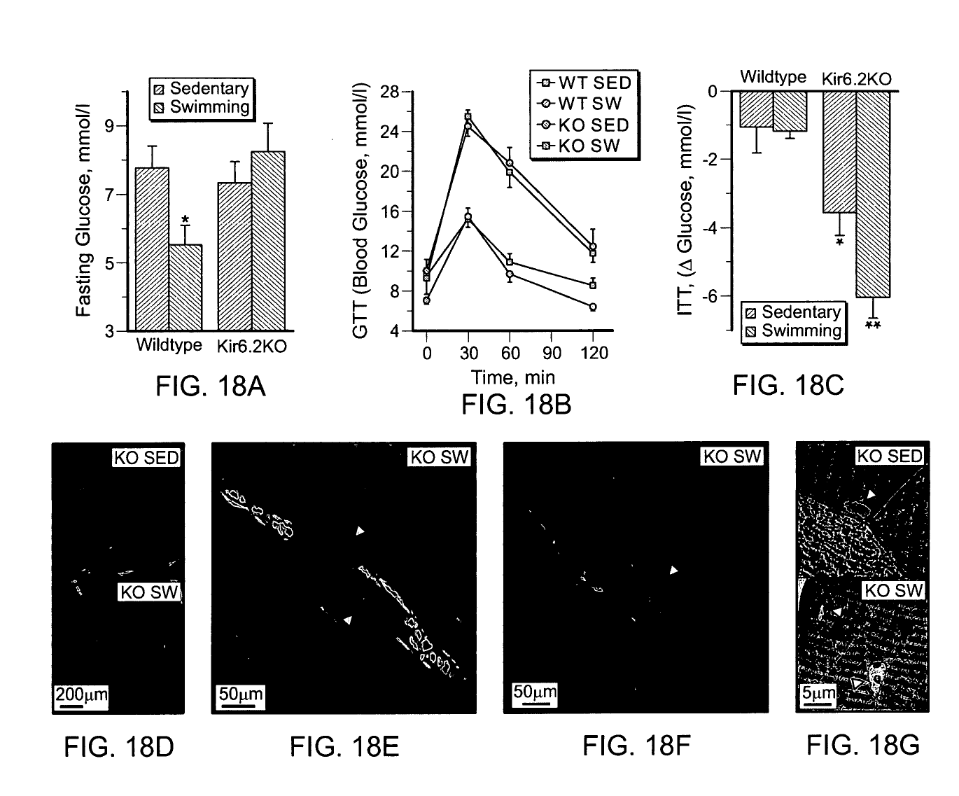

Swimming training elicited a significant reduction in fasting blood glucose in wild-type mice (−29±7% from wild-type sedentary controls, P<0.02; FIG. 18A), an effect not experienced in mice lacking K_{ATP} channels (12±12% from Kir6.2-KO sedentary controls, P=0.35; FIG. 18A). Furthermore, all wild-type mice had a normal response to glucose tolerance test (FIG. 18B), while the glucose intolerance of sedentary Kir6.2-KO mice (Miki et al., 1998, supra) was not improved by exercise training (FIG. 18B). Swimming training in wild-type did not affect the glucose response to insulin tolerance test (FIG. 18C), whereas the abnormal exaggerated glucose-lowering effect of insulin challenge that occurs in Kir6.2-KO (Miki et al., 1998, supra) was further magnified in the swum Kir6.2-KO (FIG. 18C).

In addition to the lack of these wide-ranging metabolic benefits, participation in the swimming protocol by Kir6.2-KO was achieved only at the substantial cost of myocellular damage (FIG. 18D). In contrast to wild-type skeletal muscle that did not display signs of injury following completion of the swimming regimen, hematoxylin-eosin stained hamstring muscles of the swum Kir6.2-KO compared to sendetary counterparts showed areas of myocyte degeneration with vacuolar destruction and scattered necrosis on light microscopy (FIG. 18D, E, F). Typical for areas of damage, muscle fibers from the swum Kir6.2-KO had evidence of early regenerative changes characterized by cells with basophilic cytoplasm, large nuclei, and prominent nucleoli (FIG. 18D, E), findings that were confirmed by the presence of abnormal internalized nuclei on electron microscopy (FIG. 18G).

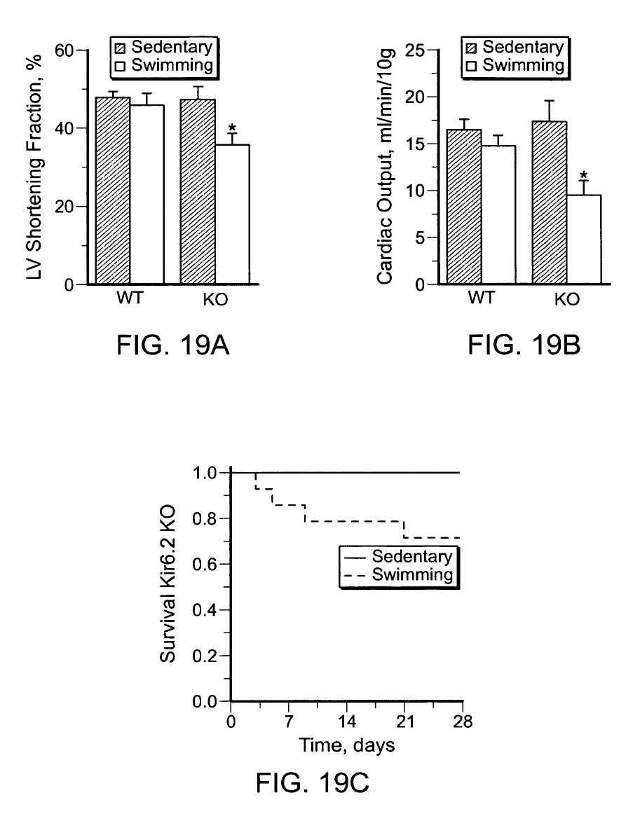

Kir6.2-KO mice that completed the swimming protocol demonstrated impaired cardiac contractile function with a significant reduction in left ventricular fractional shortening (FIG. 19A) and an impaired cardiac output on echocardiography (FIG. 19B). While in the absence of stress, mice lacking K_{ATP} channel activity had a normal survival, even the relatively modest stress imposed by the repetitive physical exertion of the swimming program induced a significant mortality in the Kir6.2-KO mice (FIG. 19C) with death occurring during or suddenly in the immediate post-exercise period. This was not seen in wild-type mice.

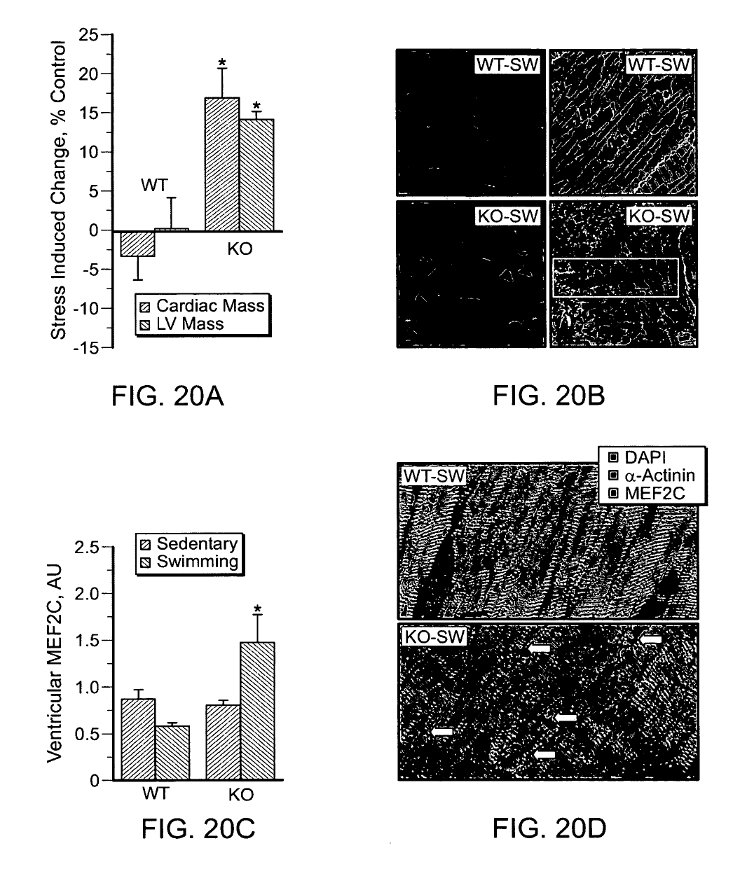

Underlying the poor cardiac contractility, Kir6.2-KO hearts following swimming were larger as measured by heart and left ventricular mass (FIG. 20A) and had pathologic evidence of myocyte damage (FIG. 20B). Unlike the wild-type (0/5; P<0.002), 5 of 6 Kir6.2-KO hearts displayed focal areas of contraction band necrosis seen on both light and electron microscopy (FIG. 20B), consistent with cytosolic calcium loading (Zingman et al., 2002, supra). Left ventricular tissue extracts taken from swum Kir6.2-KO demonstrated an increased expression of MEF2C (FIG. 20C), a critical calcium-dependent transcription factor, that when activated, translocates to the nucleus where it initiates embryonic gene reprogramming and pathologic cardiac hypertrophy. Nuclear localization of MEF2C was tested herein in left ventricular tissue by in situ immunostaining. Unlike wild-type, hearts lacking Kir6.2-containing K_{ATP} channels exhibited nuclear localization of MEF2C (FIG. 20D). Thus, K_{ATP} channel activity is required not only for both the adaptive response to exercise and the attainment of the physiologic benefits of exercise training, but also for the ability to execute these necessary responses without acquiring myocellular deficits and cardiac dysfunction.

### Part V. Kir6.2 Knockout Mice and Ventricular Dysrhythmia

Example 1—Materials & Methods

Kir6.2-knockout mice. Mice deficient in K_{ATP} channels were generated by targeted disruption of the KCNJ11 gene, which encodes the pore-forming Kir6.2 subunit of the channel complex (Miki et al., 1998, supra). Kir6.2-knockout mice were backcrossed for five generations into a C57BL/6 background. This investigation was approved by the Mayo Clinic Institutional Animal Care and Use Committee.

In situ aortic cannulation and Langendorff perfusion. Mice were anesthetized with intraperitoneal injection of 2,2,2 tri-bromoethanol (0.375 mg/g body weight; Sigma), intubated, ventilated, and the aortic root cannulated in situ (Hodgson et al., 2003, supra). Perfusion was sustained ex vivo on a Langendorff system at 90 cm H₂O, with 37° C.-prewarmed and 100% O₂-bubbled Tyrode solution (in mM: NaCl 137, KCl 5.4, CaCl₂ 2, MgCl₂ 1, HEPES 10, glucose 10; pH 7.4 with NaOH). Following 10-min equilibration, KCl was reduced to 2.7 mM and MgCl₂ to 0.35 mM, with the atrioventricular node cauterized to allow ventricular pacing. Coronary flow was monitored with a T106 blood flow meter (Transonic Systems).

Electrogram and monophasic action potential recordings. Orthogonal electrogram signals were simultaneously recorded using four silver-silver chloride electrodes surrounding the perfused heart in a simulated “Einthoven” configuration, and signals amplified by an electrocardiographic amplifier (Gould Electronics). A catheter (NuMed) was placed in the left ventricular endocardium to pace the heart at twice diastolic threshold intensity with 2 ms pulse width and 100 ms cycle length using a pulse generator (A310 Accupulser, World Precision Instruments). Monophasic action potentials were continuously recorded from the left ventricle by a probe (EP Technologies) positioned on the epicardial surface, and amplified signals (IsoDam, World Precision Instruments) acquired at 11.8 kHz and stored for off-line digital analysis.

Whole-cell patch clamp recording from isolated cardiomyocytes. Cardiomyocytes were enzymatically dissociated from the ventricular myocardium (Hodgson et al., 2003, supra). Action potentials were recorded at 30±1° C. from current-clamped isolated cells paced at 1 Hz, and superfused with Tyrode solution (pH 7.2 adjusted with KOH) using the whole-cell patch clamp technique with 5-10 MW pipettes containing (in mM) KCl 120, MgCl₂ 1, Na₂ATP 5, HEPES 10, EGTA 0.5, and CaCl₂ 0.01 (Chen et al., 2002, Basic Res. Cardiol., 97:26-34).

Statistics. Comparisons were made using the Student's t-test. A significance level of 0.05 was preselected. Data are reported as mean±SEM.

Example 2—Results

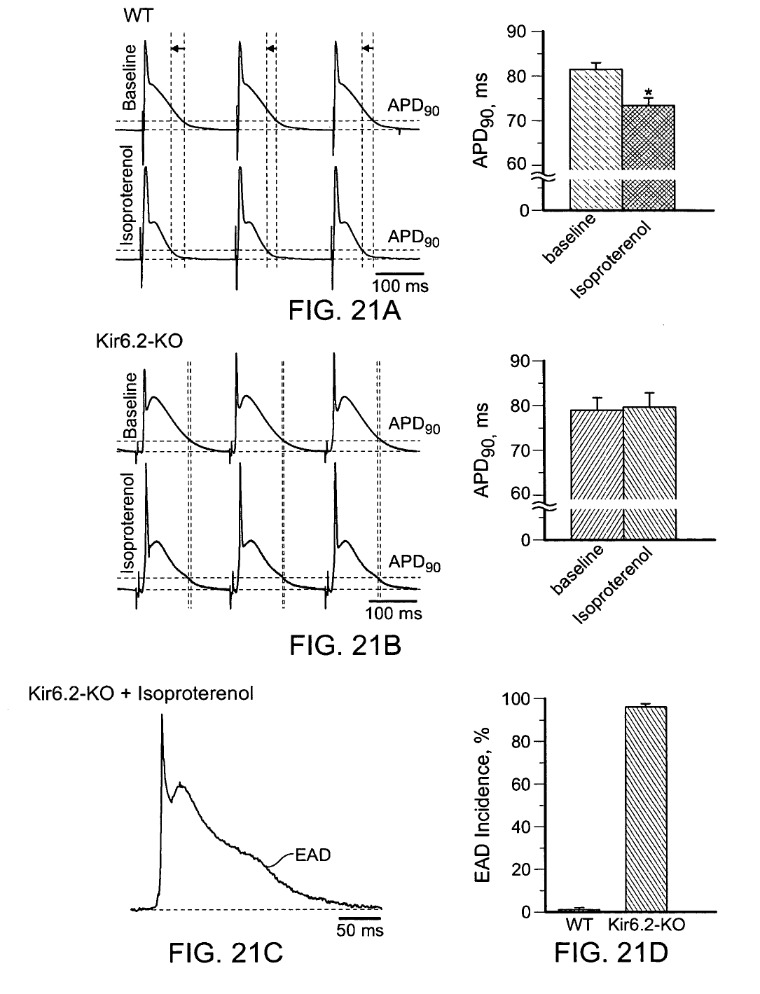

While at baseline, the action potentials were similar and the metabolic challenge of adrenergic stimulation induced distinct outcomes depending on the presence of functional K_{ATP} channels, with significant shortening of the action potential duration observed in wild-type hearts but not in age- and sex-matched counterparts lacking the Kir6.2 pore-forming channel subunit (Kir6.2-KO; FIG. 21A, B). Following a 10-min perfusion with the sympathomimetic isoproterenol (1 mM), monophasic action potential duration at 90% repolarization (APD₉₀) shortened from 82±2 ms to 74±2 ms in wild-type hearts (P<0.01, n=6; FIG. 21A). In contrast, APD₉₀ remained at 79±3 ms and 80±3 ms, prior to and following isoproterenol treatment, in Kir6.2-KO hearts (n=6; FIG. 21B). This deficit in repolarization led to distorted action potential profiles with characteristic phase 3 early afterdepolarizations manifested as distinct humps in hearts lacking functional K_{ATP} channels (FIG. 21B, C). In all Kir6.2-KO hearts (n=8), adrenergic challenge induced early afterdepolarizations, which occurred in 97±2% of action potentials examined (FIG. 21D). This is in contrast to the action potential profile of the wild-type (n=6), which maintained a smooth repolarization contour following isoproterenol challenge (FIG. 21A) without significant afterdepolarizations (1±1%; P<0.01 compared to Kir6.2-KO, FIG. 21D).

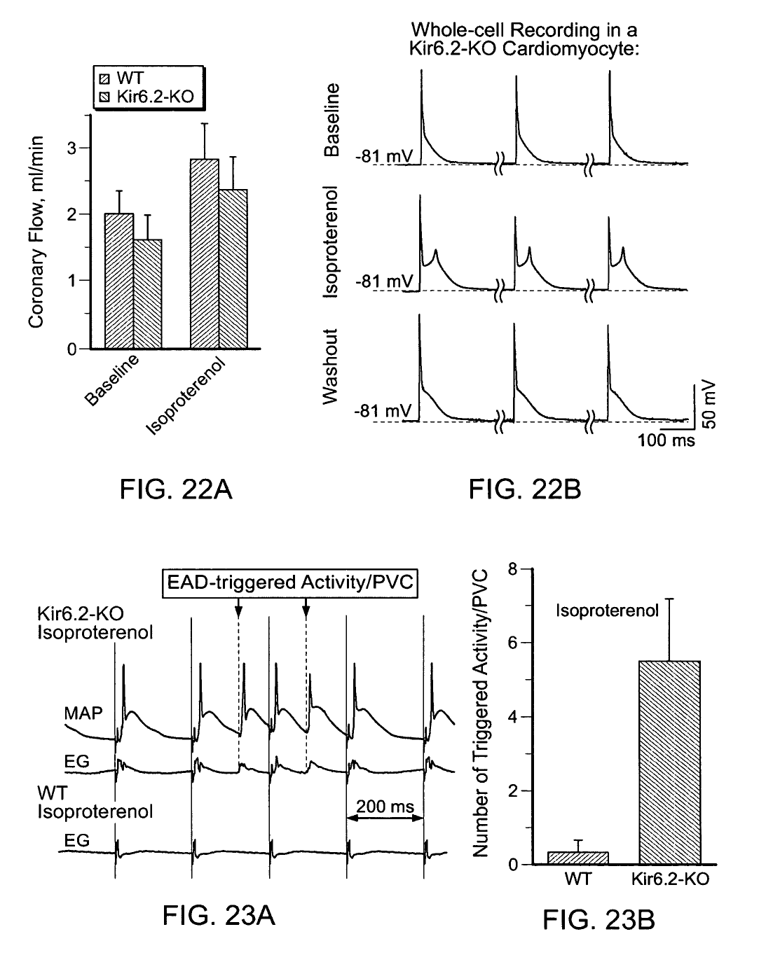

Abnormal electrical response of Kir6.2-KO hearts under adrenergic challenge was not associated with an isoproterenol-induced deficit in coronary perfusion (FIG. 22A). In fact, abnormal electrical activity during repolarization observed at the whole heart level was reproduced at the single cell level using action potential recording in isoproterenol-stressed current-clamped Kir6.2-KO cardiomyocytes (FIG. 22B).

Afterdepolarizations in isoproterenol-challenged Kir6.2-KO hearts translated into increased electrical vulnerability (FIG. 23). In the absence of functional K_{ATP} channels, afterdepolarizations induced triggered activity, disrupting regular rhythm and manifesting as premature ventricular complexes on the electrogram (FIG. 23A). On average, isoproterenol-induced afterdepolarizations complicated by triggered activity were observed in 6 out of 8 Kir6.2-KO (75%) compared to 1 out of 6 wild-type (16%) hearts, translating into a 16-fold higher risk (P<0.05) of the Kir6.2-KO mice developing premature ventricular complexes (FIG. 23B). Absence of K_{ATP} channels produces a deficit in the repolarization reserve leading to a pronounced susceptibility of Kir6.2-KO hearts to isoproterenol-induced ventricular dysrhythmia. Thus, sarcolemmal K_{ATP} channels provide for membrane electrical stability reducing the risk for arrhythmia under hyperadrenergic conditions.

### VI. K_{ATP} Channel Mutation in Adrenergic Atrial Fibrillation Originating from Vein of Marshall

Example 1—Case Report

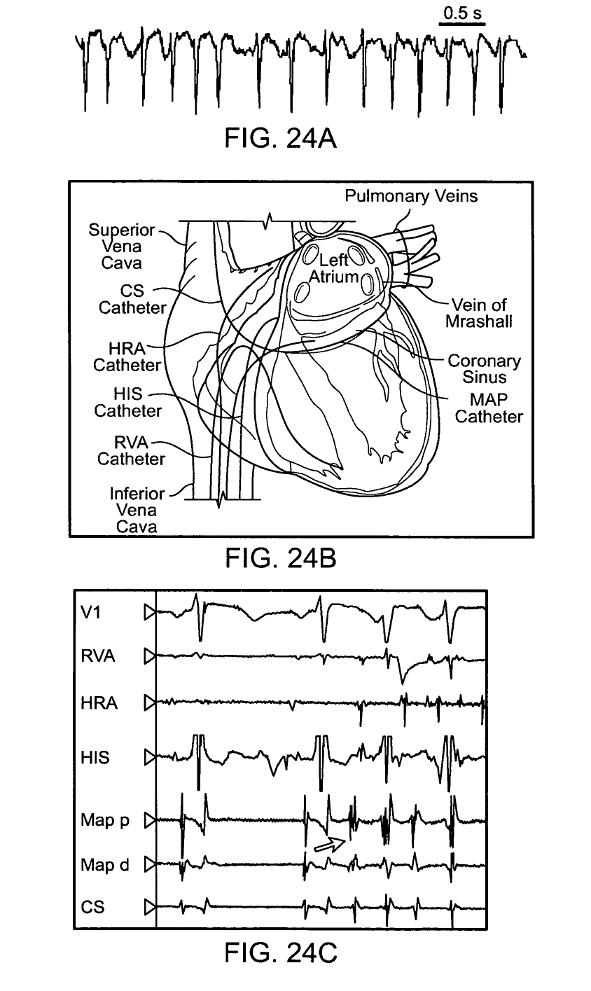

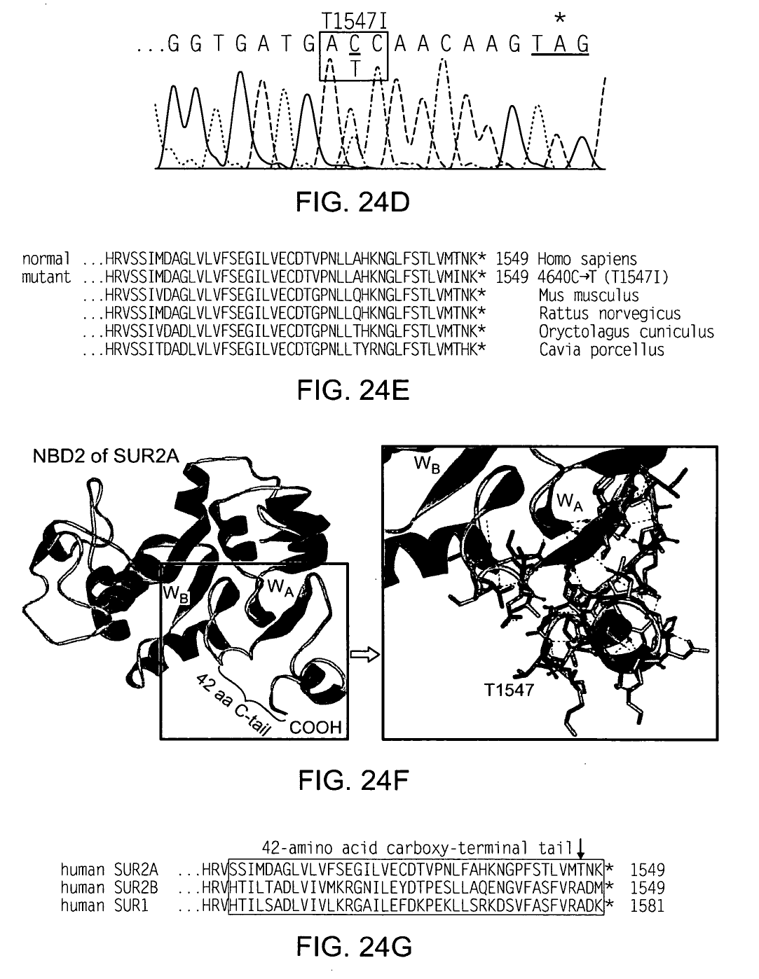

A 53-year-old female was referred for a 10-year history of daily paroxysms of atrial fibrillation peaking in morning hours with activity, and increasing in frequency with recent near syncope episodes. The patient was refractory and/or intolerant to antiarrhythmic medical therapy with flecainide, propafenone or sotalol, and use of the later drug complicated by symptomatic bradycardia while in sinus rhythm. There were no traditional risk factors for atrial fibrillation. Diagnosis of lone, adrenergically-mediated, atrial fibrillation was confirmed by documenting recurrent initiation of atrial arrhythmia under sympathomimetic challenge during continuous electrocardiographic monitoring (FIG. 24A) in the setting of a structurally normal heart by echocardiography and normal coronary perfusion stress imaging. Flurries of isoproterenol-induced atrial fibrillation were mapped to a rapidly discharging initiating focus of electrical activity within the vein of Marshall, identified by invasive electrophysiology (FIGS. 24B and 24C).

Example 2—Materials & Methods

Mutation Analysis of Genomic DNA

The study conformed to the guidelines of the Mayo Clinic Institutional Review Board, and the patient was enrolled in the study following informed written consent. Relatives declined clinical and genetic screening, precluding family assessment of cardiac rhythm and mutation status. Unrelated consenting individuals (n=2,000), from a population-based sample, served as controls. Genomic DNA, extracted from the peripheral white blood cell fraction of anticoagulated blood (Puregene, Gentra Systems), was subjected to mutation scanning of cardiac K_{ATP} channel genes by denaturing high-performance liquid chromatography heteroduplex analysis (WAVE DNA Fragment Analysis System, Transgenomic). Primers for exon-specific PCR amplification were designed using the OLIGO v6.51 Primer Analysis Software (National Biosciences) and the WAVEMAKER version 4.0.32 Software (Transgenomic). Heterozygous mutation detection was demonstrated by heteroduplex formation of PCR-amplified DNA fragments, and confirmed by cycle sequencing.

Structural Analysis of the K_{ATP} Channel SUR2A

Regional comparison of the human K_{ATP} channel SUR2A protein, encoded by ABCC9 with respective orthologs and isoforms, was made by alignment of protein sequences. The corresponding 3D model of the SUR2A carboxy-terminus was generated by the homology modeling program MODELLER 6 using as a template the crystal structure of the human TAP1 ATP-Binding Cassette protein with ADP in the binding pocket (PDB Code: 1JJ7) identified by the FASTA search (Bienengraeber et al., 2004, Nature Genet., 36:382-7). The initial atomic 3D model was refined by energy minimizations and a 2.0 ns (1.0 fs time step) molecular dynamics simulation using the AMBER 5.0/6.0 program with a second generation force field (parm96.dat) according to established protocols (Bienengraeber et al., supra).

Reconstitution of Mutant K_{ATP} Channel

Recombinant K_{ATP} channel cDNAs were subcloned into the pcDNA3.1 expression vector, and PCR-based site-directed mutagenesis (QuickChange, Stratagene) was used to replicate the disease mutation. Constructs were sequenced to verify proper introduction of the mutation, and to rule out additional changes in sequence. Human embryonic kidney (HEK293) cells cultured at 5% CO₂ in Dulbecco's Modified Eagles Medium with 10% fetal calf serum and 2 mM glutamine were co-transfected with 0.5 μg reporter green fluorescent protein and 6 μg total plasmid DNA using 15 μl Fugene (Roche). The patch-clamp technique in the inside-out configuration was applied using patch electrodes filled with 140 mM KCl, 1 mM CaCl₂, 1 mM MgCl₂ and 5 mM HEPES (pH 7.3), and cells superfused with 140 mM KCl, 1 mM MgCl₂, 5 mM EGTA and 5 mM HEPES-KOH (pH 7.3). K_{ATP} channel activity was measured at a holding potential of +60 mV and at 31±1° C. using a temperature controller (HCC-100A; Dagan Corporation). See, for example, Bienengraeber et al., supra.

Rhythm Interrogation in a K_{ATP} Channel Knockout Model

K_{ATP} channel knockout mice were backcrossed for 5 generations to a C57BL/6 background. With approval of the Mayo Foundation Institutional Animal Care and Use Committee, the heart rate and electrocardiogram were continuously monitored in the conscious state. To this end, K_{ATP} channel knockout or 8- to 12-week old, age- and sex-matched C57BL/6 wild-type mice were implanted with telemetry devices (Data Sciences International) and leads tunneled subcutaneously under isoflurane anesthesia. Following a 2-week recovery from surgery, serial signals were acquired at 2 kHz before and during isoproterenol challenge.

Mapping and Radiofrequency Ablation of the Atrial Fibrillation Initiation Focus

Light sedation was administered to the patient in the fasting state. Following heparinization, femoral and internal jugular venous sheaths were introduced for placement of intra-cardiac monitoring catheters, positioned in the right atrium, coronary sinus, right ventricle, bundle of His, and following trans-septal puncture left atrium. Multi-polar catheters were placed through trans-septal sheaths for electrical interrogation of the pulmonary veins, and an additional mapping catheter advanced through the coronary sinus into the vein of Marshall. An intracardiac ultrasound probe was placed into the right atrium to guide catheter localization to the veno-atrial junctures. Focal initiation sites for atrial fibrillation were mapped by a roving catheter and differential pacing. Electrical activation was recorded at baseline and under stress provocation. Adrenergic stimulation was carried out by intravenous infusion of isoproterenol at 2 μg/min. Ablation of mapped atrial fibrillation-initiating foci was undertaken by applying radiofrequency energy.

Example 3—Results

K_{ATP} Channel Mutation in Atrial Fibrillation

Following the diagnosis of lone adrenergic atrial fibrillation and mapping of the initiation site to the vein of Marshall (FIG. 24A-C), the patient underwent genetic evaluation. Scanning of genomic DNA by heteroduplex mutational analysis demonstrated an anomaly in ABCC9, the gene encoding the regulatory SUR2A module of the K_{ATP} channel complex. The anomaly was identified in exon 38, specific for the cardiac splice variant of SUR2A. DNA sequencing revealed a heterozygous 4640C>T missense mutation causing substitution of the 1547 threonine residue with isoleucine (FIG. 24D). To eliminate the possibility that the identified T1547I substitution is a polymorphism within the normal population, 2,000 unrelated control individuals were tested and all 4,000 chromosomes scanned were found negative for the ABCC9 gene mutation. The T15471 substitution altered the amino-acid sequence of the evolutionarily conserved carboxy-terminal domain of the SUR2A protein (FIG. 24E). This tail region is in proximity to the signature Walker motifs (W_{A} and W_{B}) of the nucleotide binding domain (NBD2) required for coordination of nucleotides in the nucleotide binding pocket (FIG. 24F). The carboxy-terminal tail including the Thr1547 residue is structurally and functionally unique to SUR2A, distinguishing this isoform from non-cardiac K_{ATP} channel SUR proteins (FIG. 24G). Removal of the polar threonine (Thr1547) and replacement with the larger, aliphatic and highly hydrophobic isoleucine as would occur in this patient predicts compromised K_{ATP} channel function based on an aberrant structure of SUR2A.

K_{ATP} Channel Dysfunction and Atrial Fibrillation

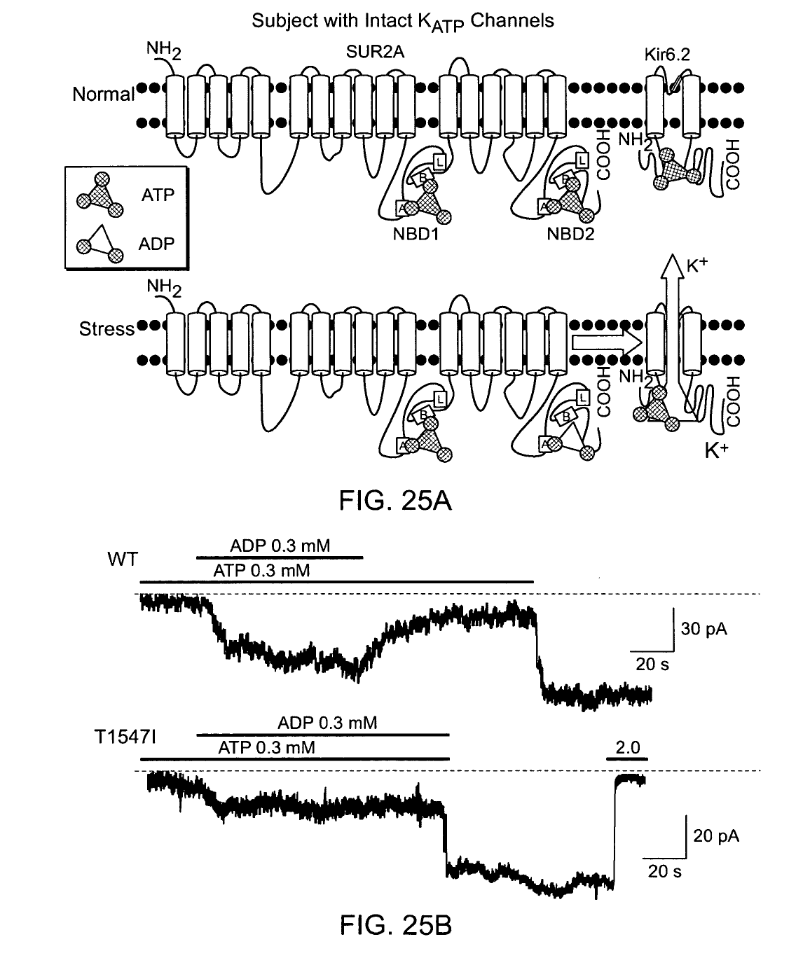

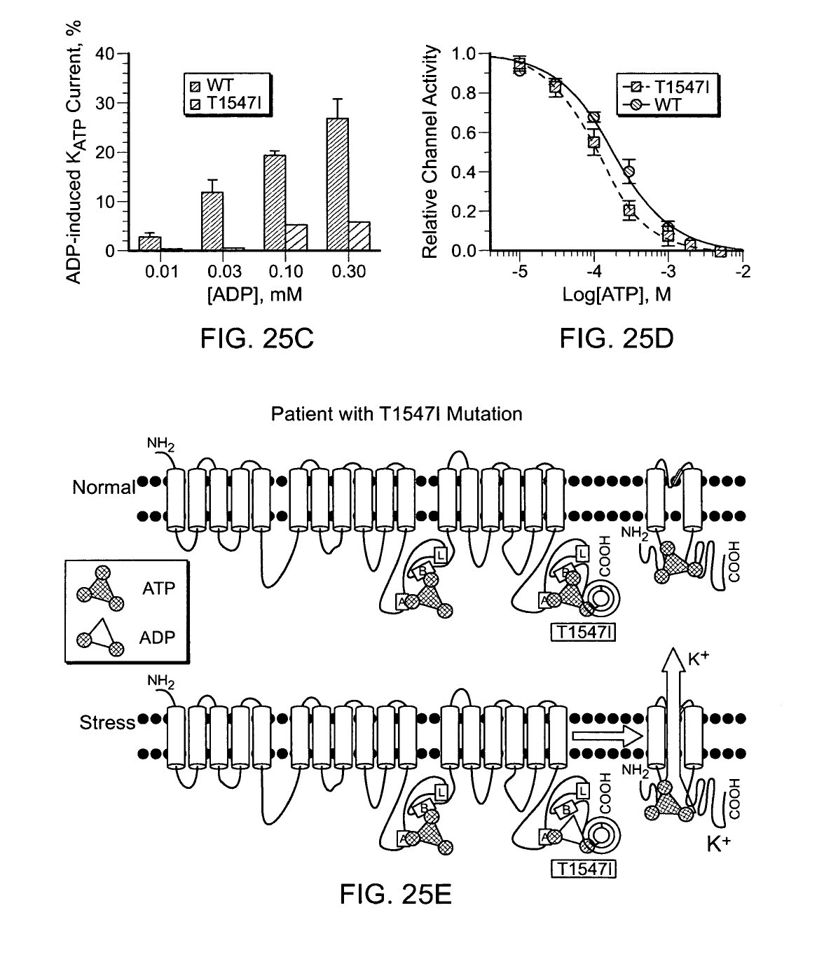

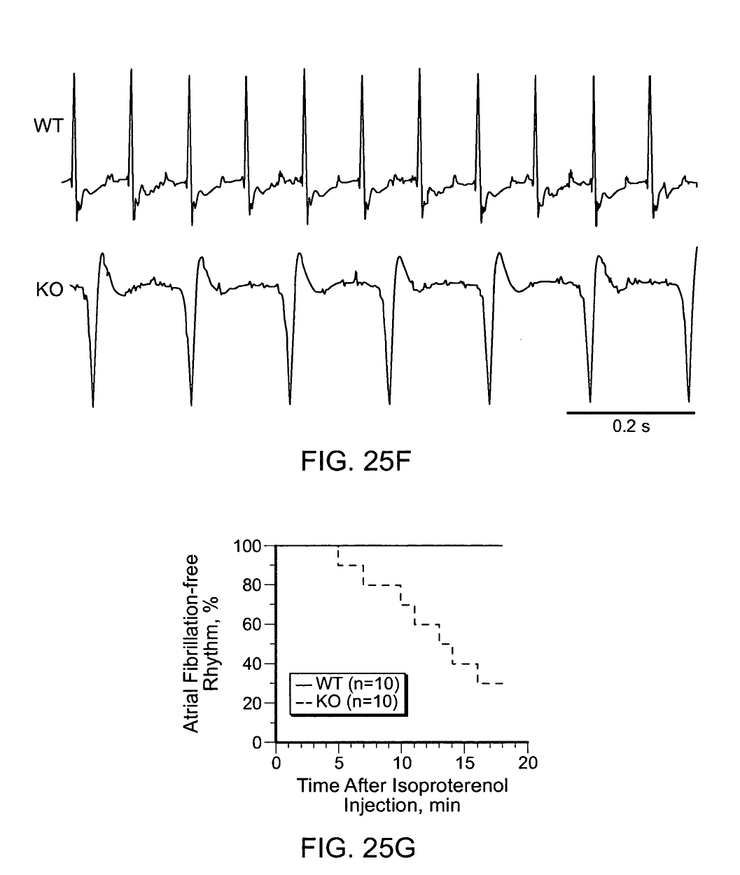

The cardiac K_{ATP} channel complex is formed by physical association of the regulatory SUR2A subunit, that harbors nucleotide binding domains, and Kir6.2, the conduit for potassium ions (FIG. 25A). The reconstituted T15471 substitution in SUR2A compromised adenine nucleotide-dependent induction of K_{ATP} channel current, as demonstrated in a cellular heterologous expression system (FIG. 25B). Specifically, mutant T15471 SUR2A co-expressed with the Kir6.2 pore generated a functional channel that retained ATP-induced inhibition of potassium current but displayed an aberrant phenotype with a dramatically reduced responsiveness towards ADP, the end-product of metabolic stress (FIG. 25C-25E). Deficit in SUR2A-dependent nucleotide gating of the K_{ATP} channel resulting from the T1547I mutation would thus compromise the homeostatic role of the channel complex required for proper readout of cellular distress and maintenance of electrical stability. The pathogenic link between K_{ATP} channel dysfunction and atrial fibrillation was verified at the whole organism level in a murine knockout model deprived of operational channels. Compared to the normal atrium that was resistant to arrhythmia under adrenergic provocation, vulnerability to atrial fibrillation was recapitulated in the setting of K_{ATP} channel deficit (FIGS. 25F and 25G).

Restoration of Sinus Rhythm

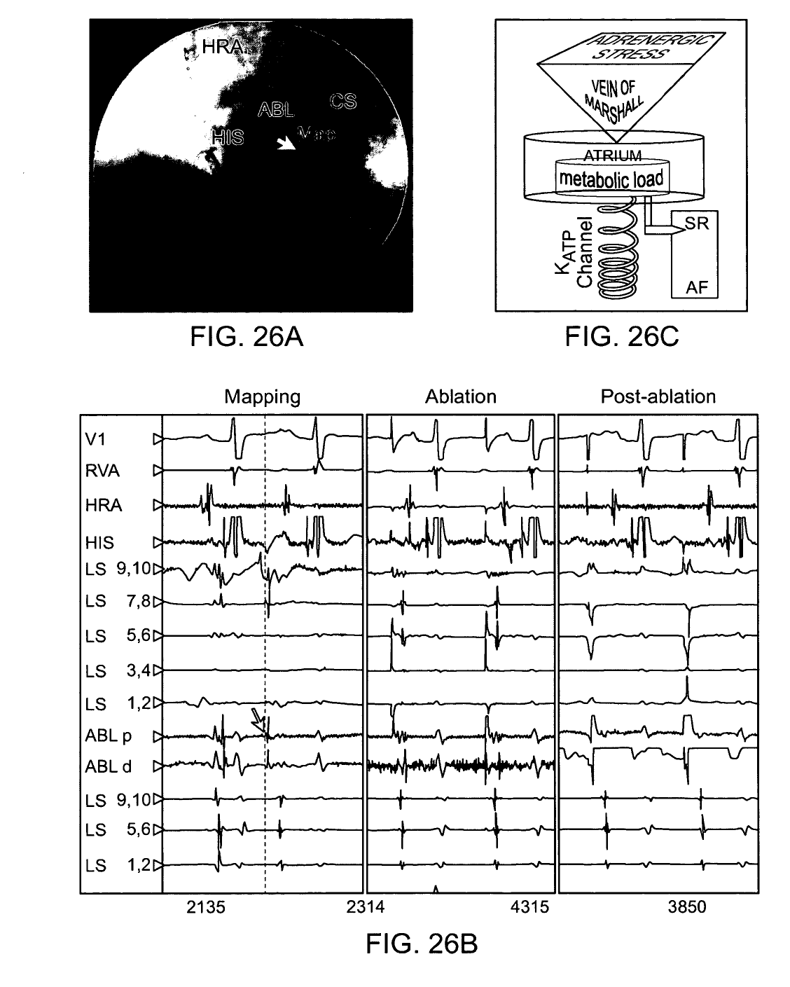

In the patient, refractoriness of atrial fibrillation to medical therapy was an indication for ablative intervention to restore sinus rhythm. Following mapping of the focus of catecholamine-inducible atrial ectopy, delivery of radiofrequency energy was targeted to the earliest activation site within the left atrium overlying the vein of Marshall (FIGS. 26A and 26B). Endocardial ablation eliminated atrial ectopy (FIG. 26B). Following ablation, atrial fibrillation could no longer be provoked by program electrical stimulation or by burst pacing with or without isoproterenol infusion (FIG. 26B). Post-ablation, the patient has remained in sinus rhythm and symptom-free over a 2.5 year follow-up period.

### VII. Polymorphism in Kir6.2 of the K_{ATP} Channel Confers Susceptibility to Increased Left Ventricular Size in the Population

A single nucleotide polymorphism (67A>G) in human Kir6.2 results in two forms of the Kir6.2 protein, which are distinguished by either a lysine or a glutamic acid at amino acid residue 23 (E23K). To test this hypothesis, genotype-phenotype analyses of a population-based cross-sectional cohort of 2,050 adults in Olmsted County, Minn., were performed. The cohort is comprised of patients whose cardiac phenotype has been characterized by echocardiography and from whom blood has been collected for DNA analyses. DNA sequence flanking the 67A>G variant was amplified by the polymerase chain reaction, and then digested with the BanII restriction enzyme. Resultant fragments that varied in size were resolved on agarose gels, which enabled the assignment of genotype: E/E=126 bp fragments, E/K=126 bp+154 bp fragments, and KK=154 bp fragments.

A statistically significant association between the more rare K allele and increased left ventricular dimensions was demonstrated in the subsets of patients with hypertension and metabolic syndrome. Hypertension and metabolic syndrome are conditions that place increased stress load on the heart. These findings demonstrate a broader significance of the K_{ATP} channel in cardiac health and disease in the population at large.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying an individual at risk for cardiac disease, wherein said method comprises:
determining whether or not said individual contains a mutation present in a nucleic acid encoding a SUR2A polypeptide or regulating the expression of said SUR2A polypeptide, wherein the presence of said mutation indicates that said individual is at risk for cardiac disease.

2. The method of claim 1, wherein said cardiac disease is heart failure, ventricular arrhythmias, or atrial arrhythmias.

3. The method of claim 1, wherein said individual is a human.

4. The method of claim 1, wherein said mutation present in said nucleic acid encoding said SUR2A polypeptide results in Fs1524, Ala1513Thr, or Thr15471Ile.

5. The method of claim 1, wherein said mutation is detected by sequencing, electrophoretic mobility, nucleic acid hybridization, fluorescent in situ hybridization, polymerase chain reaction, reverse transcription-polymerase chain reaction, denaturing high-performance liquid chromatography, or a combination thereof.

6. A method for identifying an individual who will not respond to, or who is not responding to, conventional treatment of sepsis or hypertension, wherein said method comprises:
determining whether or not said individual contains a mutation present in a nucleic acid encoding a SUR2A polypeptide or regulating the expression of said SUR2A polypeptide, wherein the presence of said mutation indicates that said individual is identified as one who will not respond to or who is not responding to conventional treatment of sepsis or hypertension.

7. The method of claim 6, further comprising:
modifying the treatment of said individual for sepsis or hypertension based on said identification.

8. A method for screening an individual for a deficiency in cardiac K_{ATP} channeling, wherein said individual has experienced a poor outcome with catecholamine induction, wherein said method comprises:
determining whether or not said individual contains a mutation present in a nucleic acid encoding a SUR2A polypeptide or regulating the expression of a SUR2A polypeptide, wherein the presence of said mutation indicates that said individual is identified as having a deficiency in cardiac K_{ATP} channeling.

9. A method of treating an individual having a deficiency in cardiac K_{ATP} channeling, wherein said method comprises:
identifying an individual as having said deficiency; and
administering an effective dose of a potassium channel opener (PCO) to said individual, wherein said PCO improves cardiac KATP channeling, thereby treating the individual for the deficiency in cardiac K_{ATP} channeling.

10. The method of claim 9, further comprising:
monitoring said individual for cardiac K_{ATP} channeling.

11. The method of claim 10, further comprising:
administering a calcium channel blocker if the individual does not respond to the PCO.

12. An isolated nucleic acid comprising a sequence encoding a mammalian SUR2A polypeptide, wherein said sequence comprises a mutation that disrupts the function of an assembled K_{ATP} channel.

13. The isolated nucleic acid of claim 12, wherein said mutation results in a frameshift at the codon encoding Leucine 1524 (Fs1524) in the reference sequence set forth in SEQ ID NO:11.

14. The isolated nucleic acid of claim 13, wherein said frameshift is due to a 3 basepair deletion and a 4 basepair insertion at position 4570-4572 (4570-4572delTTAinsAAAT) in the reference sequence set forth in SEQ ID NO:10.

15. The isolated nucleic acid of claim 14, said nucleic acid comprising the nucleotide sequence set forth in SEQ ID NO:12.

16. The isolated nucleic acid of claim 12, wherein said mutation results in an amino acid substitution of Thr to Ala at position 1513 (Ala1513Thr) in the reference sequence set forth in SEQ ID NO:11.

17. The isolated nucleic acid of claim 16, wherein said substitution is caused by a G to A missense mutation at position 4537 in the reference sequence set forth in SEQ ID NO:12.

18. The isolated nucleic acid of claim 17, said nucleic acid comprising the nucleotide sequence set forth in SEQ ID NO:14.

19. A substantially pure mammalian SUR2A polypeptide, wherein said polypeptide comprises a mutation that disrupts the function of an assembled K_{ATP} channel.

20. The polypeptide of claim 19, wherein said mutation reflects a frameshift in the nucleic acid encoding said SUR2A polypeptide at the codon encoding Leu1524.

21. The polypeptide of claim 20, wherein said polypeptide comprises the amino acid sequence set forth in SEQ ID NO:13.

22. The polypeptide of claim 19 wherein said mutation is an Ala1513Thr substitution.

23. The polypeptide of claim 22, wherein said polypeptide comprises the amino acid sequence set forth in SEQ ID NO:15.

24. A nucleic acid construct comprising the nucleic acid of claim 12.

25. A host cell comprising the nucleic acid construct of claim 24.

26. An antibody that binds to the polypeptide of claim 19 and does not bind to a polypeptide having an aimino acid sequence as set forth in SEQ ID NO:11.

27. The antibody of claim 26, wherein said antibody is selected from the group consisting of a monoclonal antibody, a humanised antibody, a chimaeric antibody, an antibody fragment, a single chain antibody, and a single domain antibody.

28. The antibody of claim 27, wherein said antibody fragment is a Fab fragment, a (Fab′)₂ fragment, or a Fv fragment.


SEQUENCE LISTING (26 sequences)

SEQ ID NO: 1 - DNA, 25 bp, Artificial Sequence
  Oligonucleotide
agatacccacaacacaccacgcgcc

SEQ ID NO: 2 - DNA, 25 bp, Artificial Sequence
  Oligonucleotide
atccttcagagagtcgcatgcgctt

SEQ ID NO: 3 - protein, 45 aa, Homo sapiens
HRVSSIMDAGLVLVFSEGILVECDTVPNLLAHKNGLFSTLVMTNK

SEQ ID NO: 4 - protein, 23 aa, Homo sapiens
HRVSSIMDAGLVLVFSEGIKCGV

SEQ ID NO: 5 - protein, 45 aa, Homo sapiens
HRVSSIMDTGLVLVFSEGILVECDTVPNLLAHKNGLFSTLVMTNK

SEQ ID NO: 6 - protein, 45 aa, Rattus norvegicus
HRVSSIMDAGLVLVFSEGILVECDTGPNLLQHKNGLFSTLVMTNK

SEQ ID NO: 7 - protein, 45 aa, Mus musculus
HRVSSIVDAGLVLVFSEGILVECDTGPNLLQHKNGLFSTLVMTNK

SEQ ID NO: 8 - protein, 45 aa, Oryctolagus cuniculus
HRVSSIVDADLVLVFSEGILVECDTGPNLLTHKNGLFSTLVMTNK

SEQ ID NO: 9 - protein, 45 aa, Cavia porcellus
HRVSSITDADLVLVFSEGILVECDTGPNLLTYRNGLFSTLVMTHK

SEQ ID NO: 10 - DNA, 4650 bp, Homo sapiens
atgagcctttcattttgtggtaacaacatttcttcatataatatcaacgatggtgtactacaaaattcctgctttgtggatgccctcaacctggtccctcatgtctttctgttgtttatcacttttccaatattgtttattgggtgggggagccaaagctcaaaagtacaaattcaccacaacacatggcttcattttccgggacataacctgagatggattcttacattcgctctcctgtttgtgcatgtctgtgaaatagcagaaggcattgtttcagactcgcggcgggaatcaaggcacctccacctctttatgccagccgtgatgggattcgttgccactacaacatcgatagtgtattatcataatatcgaaacatcaaattttcctaaattacttttagccctgttcctgtattgggtaatggcctttattacaaaaacaataaaattggttaagtactgtcagtctggcttggacatatcaaacctgcgtttctgcatcacaggcatgatggtcatcttgaatgggctcttgatggctgtggagatcaatgtcattcgagtcaggagatatgtatttttcatgaatcctcagaaagtaaagcctcctgaagacctccaggatctgggagtgagatttcttcaaccatttgtgaatttgctgtcaaaagcaacatactggtggatgaacacacttattatatctgctcacaaaaagcctattgatctgaaggcaattggaaaattgccaatagcaatgagagcagtaacaaattatgtttgcctgaaagatgcatatgaagaacaaaagaaaaaagttgcagatcatccaaatcggactccatctatatggcttgcaatgtacagagcttttgggcgaccaattctacttagtagcacattccgctatctggctgatttactgggttttgctggacctctttgtatttctggaatagttcagcgtgtgaatgaaacccagaatgggacaaataacacaactggaatttcagaaaccctctcatcaaaggaatttcttgaaaacgcttacgttctagcagttcttctcttcttggctcttattctgcaaaggacatttttgcaggcttcctactatgtaaccatagagactggcattaacctccgtggagctctgctggccatgatttataataaaatccttaggctctctacgtctaacttatccatgggggagatgactctggggcagatcaacaacttagtcgccattgaaactaatcaactcatgtggtttttgttcctgtgtcccaatctatgggctatgcctgttcagatcataatgggcgtgattctgctctataatttacttggatcaagtgcattggtcggtgcagctgtcattgtgctccttgcgccaattcagtactttattgctacaaagttggcagaggctcagaaaagtacacttgattattccactgagagactcaagaaaacaaatgaaatattgaaaggcatcaaacttctaaaattgtatgcctgggaacacattttctgcaaaagtgtggaggaaacaagaatgaaagaactatctagtctcaaaacctttgcactatatacatcactctccatcttcatgaatgcagcaattcccatagcagctgttcttgctacatttgtgacccatgcgtatgccagtggaaacaatctgaaacctgcagaggcctttgcttcactgtctctcttccatatcctggtcacaccactgtccctgctcttcacggtggtcagatttgcagtcaaagccatcataagtgttcaaaagctgaatgagtttctcttgagtgatgagattggtgacgacagttggcgaactggtgaaagttcgcttccttttgagtcctgtaagaagcacactggagttcagccaaaaactataaacaggaaacagcctggaagatatcacctggacagctatgagcaatcaacacggcgtctacgtcccgcagaaacagaggacattgcaataaaggtcacaaatggatacttttcatggggcagtggtttagctacattatccaatatagatattcgaattccaacaggtcagttaaccatgattgtgggccaagtaggatgtgggaagtcctctcttctccttgccatcctcggtgagatgcagacattggaaggaaaagttcactggagcaatgtaaatgaatctgagccttcttttgaagcaaccagaagtaggaacaggtactctgtggcatatgcagctcaaaagccttggctattaaatgctacagtagaagaaaatattacttttggaagtccttttaacaaacagaggtacaaagctgtcacagatgcctgttctcttcagccagatattgacttattaccatttggagatcaaactgaaattggagagaggggcatcaacctgagtgggggacagaggcagagaatctgtgtggcacgagcgctgtatcaaaacaccaacattgtctttttggatgatccattctcagccctggacattcacttgagtgatcatttaatgcaggaggggattttgaaattcctgcaagatgacaaaaggacactcgttcttgtgactcacaaattacagtatctgacgcatgctgactggatcatagccatgaaagatggaagtgtcctaagagaaggaactttgaaggacattcaaaccaaagatgttgagctttatgaacactggaaaacacttatgaatcggcaagatcaagaattagaaaaggatatggaagctgaccaaactactttagagaggaaaactctccgacgggccatgtattcaagagaagccaaagcccagatggaggacgaagacgaagaggaagaagaggaggaagatgaggatgataacatgtccactgtaatgaggctcaggactaaaatgccatggaaaacctgctggcgctacctgacatctggaggattcttcctgctcatcctgatgattttctctaagcttttgaagcattcggtcattgtagctatagactattggctggccacatggacatcggagtacagtataaacaatactggaaaagctgatcagacctactatgtggctggctttagcatactctgtggagcaggcattttcctttgccttgttacatccctcactgtagaatggatgggtctcacagctgccaaaaatcttcaccacaaccttctcaataagataatccttggaccaataaggttttttgataccacacccctgggactgattctcaatcgcttttcagctgatactaatatcattgatcagcacatccctccaaccttggaatctctaactcgctcaacactgctctgcctgtctgccattgggatgatttcttatgctactcctgtgttcctggttgctctcctgccccttggtgttgccttttattttatccagaaatactttcgggttgcctctaaggacctccaggaactcgacgatagtacccagctccctctgctctgtcacttctcagaaacagcagaaggactcaccaccattcgggcctttaggcatgaaaccagatttaaacaacgtatgctggaactgacggatacaaacaacattgcctacttatttctctcagctgccaacagatggctggaggtcaggacggattatctgggagcttgcattgtcctcactgcatctatagcatccattagtgggtcttccaattctggattggtaggcttgggtcttctgtatgcacttacgataaccaattatttgaattgggttgtgaggaacttggctgacctggaggtccagatgggtgcagtgaagaaggtgaacagtttcctgactatggagtcagagaactatgaaggcacaatggatccttctcaagttccagaacattggccacaagaaggggagatcaagatacatgatctgtgtgtcagatatgaaaataatctgaaacctgttcttaagcacgtcaaggcttacatcaaacctggacaaaaggtgggcatatgtggtcgcactggcagtgggaaatcatcgttatctctggctttcttcagaatggttgatatatttgatggaaaaattgtcattgatgggatagacatttccaaattaccactgcacacactacgttctagactttcaatcattctgcaggatccaatactattcagtggttccattagatttaatttagatccagagtgcaaatgcacagatgacagactctgggaagccttagaaattgctcagctgaagaatatggtcaaatctctacctggaggtctagatgcggttgtcactgaaggtggggagaattttagcgtgggacagagacagctattttgccttgccagggcctttgtccgcaaaagcagcattcttattatggatgaggcaacagcttccattgacatggccacagagaatattttgcaaaaagtagtaatgacagcctttgcagaccggaccgtggtgacaatggctcaccgtgtctcttctattatggatgcaggccttgttttagtcttttctgagggtattttagtggagtgtgatactgtcccaaatttgttcgcccacaagaatggccccttttccactttggtgatgaccaacaagtag

SEQ ID NO: 11 - protein, 1549 aa, Homo sapiens
MSLSFCGNNISSYNINDGVLQNSCFVDALNLVPHVFLLFITFPILFIGWGSQSSKVQIHHNTWLHFPGHNLRWILTFALLFVHVCEIAEGIVSDSRRESRHLHLFMPAVMGFVATTTSIVYYHNIETSNFPKLLLALFLYWVMAFITKTIKLVKYCQSGLDISNLRFCITGMMVILNGLLMAVEINVIRVRRYVFFMNPQKVKPPEDLQDLGVRFLQPFVNLLSKATYWWMNTLIISAHKKPIDLKAIGKLPIAMRAVTNYVCLKDAYEEQKKKVADHPNRTPSIWLAMYRAFGRPILLSSTFRYLADLLGFAGPLCISGIVQRVNETQNGTNNTTGISETLSSKEFLENAYVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSIFMNAAIPIAAVLATFVTHAYASGNNLKPAEAFASLSLFHILVTPLSLLFTVVRFAVKAIISVQKLNEFLLSDEIGDDSWRTGESSLPFESCKKHTGVQPKTINRKQPGRYHLDSYEQSTRRLRPAETEDIAIKVTNGYFSWGSGLATLSNIDIRIPTGQLTMIVGQVGCGKSSLLLAILGEMQTLEGKVHWSNVNESEPSFEATRSRNRYSVAYAAQKPWLLNATVEENITFGSPFNKQRYKAVTDACSLQPDIDLLPFGDQTEIGERGINLSGGQRQRICVARALYQNTNIVFLDDPFSALDIHLSDHLMQEGILKFLQDDKRTLVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWRYLTSGGFFLLILMIFSKLLKHSVIVAIDYWLATWTSEYSINNTGKADQTYYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLVALLPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDAVVTEGGENFSVGQRQLFCLARAFVRKSSILIMDEATASIDMATENILQKVVMTAFADRTVVTMAHRVSSIMDAGLVLVFSEGILVECDTVPNLFAHKNGPFSTLVMTNK

SEQ ID NO: 12 - DNA, 4584 bp, Homo sapiens
atgagcctttcattttgtggtaacaacatttcttcatataatatcaacgatggtgtactacaaaattcctgctttgtggatgccctcaacctggtccctcatgtctttctgttgtttatcacttttccaatattgtttattgggtgggggagccaaagctcaaaagtacaaattcaccacaacacatggcttcattttccgggacataacctgagatggattcttacattcgctctcctgtttgtgcatgtctgtgaaatagcagaaggcattgtttcagactcgcggcgggaatcaaggcacctccacctctttatgccagccgtgatgggattcgttgccactacaacatcgatagtgtattatcataatatcgaaacatcaaattttcctaaattacttttagccctgttcctgtattgggtaatggcctttattacaaaaacaataaaattggttaagtactgtcagtctggcttggacatatcaaacctgcgtttctgcatcacaggcatgatggtcatcttgaatgggctcttgatggctgtggagatcaatgtcattcgagtcaggagatatgtatttttcatgaatcctcagaaagtaaagcctcctgaagacctccaggatctgggagtgagatttcttcaaccatttgtgaatttgctgtcaaaagcaacatactggtggatgaacacacttattatatctgctcacaaaaagcctattgatctgaaggcaattggaaaattgccaatagcaatgagagcagtaacaaattatgtttgcctgaaagatgcatatgaagaacaaaagaaaaaagttgcagatcatccaaatcggactccatctatatggcttgcaatgtacagagcttttgggcgaccaattctacttagtagcacattccgctatctggctgatttactgggttttgctggacctctttgtatttctggaatagttcagcgtgtgaatgaaacccagaatgggacaaataacacaactggaatttcagaaaccctctcatcaaaggaatttcttgaaaacgcttacgttctagcagttcttctcttcttggctcttattctgcaaaggacatttttgcaggcttcctactatgtaaccatagagactggcattaacctccgtggagctctgctggccatgatttataataaaatccttaggctctctacgtctaacttatccatgggggagatgactctggggcagatcaacaacttagtcgccattgaaactaatcaactcatgtggtttttgttcctgtgtcccaatctatgggctatgcctgttcagatcataatgggcgtgattctgctctataatttacttggatcaagtgcattggtcggtgcagctgtcattgtgctccttgcgccaattcagtactttattgctacaaagttggcagaggctcagaaaagtacacttgattattccactgagagactcaagaaaacaaatgaaatattgaaaggcatcaaacttctaaaattgtatgcctgggaacacattttctgcaaaagtgtggaggaaacaagaatgaaagaactatctagtctcaaaacctttgcactatatacatcactctccatcttcatgaatgcagcaattcccatagcagctgttcttgctacatttgtgacccatgcgtatgccagtggaaacaatctgaaacctgcagaggcctttgcttcactgtctctcttccatatcctggtcacaccactgtccctgctcttcacggtggtcagatttgcagtcaaagccatcataagtgttcaaaagctgaatgagtttctcttgagtgatgagattggtgacgacagttggcgaactggtgaaagttcgcttccttttgagtcctgtaagaagcacactggagttcagccaaaaactataaacaggaaacagcctggaagatatcacctggacagctatgagcaatcaacacggcgtctacgtcccgcagaaacagaggacattgcaataaaggtcacaaatggatacttttcatggggcagtggtttagctacattatccaatatagatattcgaattccaacaggtcagttaaccatgattgtgggccaagtaggatgtgggaagtcctctcttctccttgccatcctcggtgagatgcagacattggaaggaaaagttcactggagcaatgtaaatgaatctgagccttcttttgaagcaaccagaagtaggaacaggtactctgtggcatatgcagctcaaaagccttggctattaaatgctacagtagaagaaaatattacttttggaagtccttttaacaaacagaggtacaaagctgtcacagatgcctgttctcttcagccagatattgacttattaccatttggagatcaaactgaaattggagagaggggcatcaacctgagtgggggacagaggcagagaatctgtgtggcacgagcgctgtatcaaaacaccaacattgtctttttggatgatccattctcagccctggacattcacttgagtgatcatttaatgcaggaggggattttgaaattcctgcaagatgacaaaaggacactcgttcttgtgactcacaaattacagtatctgacgcatgctgactggatcatagccatgaaagatggaagtgtcctaagagaaggaactttgaaggacattcaaaccaaagatgttgagctttatgaacactggaaaacacttatgaatcggcaagatcaagaattagaaaaggatatggaagctgaccaaactactttagagaggaaaactctccgacgggccatgtattcaagagaagccaaagcccagatggaggacgaagacgaagaggaagaagaggaggaagatgaggatgataacatgtccactgtaatgaggctcaggactaaaatgccatggaaaacctgctggcgctacctgacatctggaggattcttcctgctcatcctgatgattttctctaagcttttgaagcattcggtcattgtagctatagactattggctggccacatggacatcggagtacagtataaacaatactggaaaagctgatcagacctactatgtggctggctttagcatactctgtggagcaggcattttcctttgccttgttacatccctcactgtagaatggatgggtctcacagctgccaaaaatcttcaccacaaccttctcaataagataatccttggaccaataaggttttttgataccacacccctgggactgattctcaatcgcttttcagctgatactaatatcattgatcagcacatccctccaaccttggaatctctaactcgctcaacactgctctgcctgtctgccattgggatgatttcttatgctactcctgtgttcctggttgctctcctgccccttggtgttgccttttattttatccagaaatactttcgggttgcctctaaggacctccaggaactcgacgatagtacccagctccctctgctctgtcacttctcagaaacagcagaaggactcaccaccattcgggcctttaggcatgaaaccagatttaaacaacgtatgctggaactgacggatacaaacaacattgcctacttatttctctcagctgccaacagatggctggaggtcaggacggattatctgggagcttgcattgtcctcactgcatctatagcatccattagtgggtcttccaattctggattggtaggcttgggtcttctgtatgcacttacgataaccaattatttgaattgggttgtgaggaacttggctgacctggaggtccagatgggtgcagtgaagaaggtgaacagtttcctgactatggagtcagagaactatgaaggcacaatggatccttctcaagttccagaacattggccacaagaaggggagatcaagatacatgatctgtgtgtcagatatgaaaataatctgaaacctgttcttaagcacgtcaaggcttacatcaaacctggacaaaaggtgggcatatgtggtcgcactggcagtgggaaatcatcgttatctctggctttcttcagaatggttgatatatttgatggaaaaattgtcattgatgggatagacatttccaaattaccactgcacacactacgttctagactttcaatcattctgcaggatccaatactattcagtggttccattagatttaatttagatccagagtgcaaatgcacagatgacagactctgggaagccttagaaattgctcagctgaagaatatggtcaaatctctacctggaggtctagatgcggttgtcactgaaggtggggagaattttagcgtgggacagagacagctattttgccttgccagggcctttgtccgcaaaagcagcattcttattatggatgaggcaacagcttccattgacatggccacagagaatattttgcaaaaagtagtaatgacagcctttgcagaccggaccgtggtgacaatggctcaccgtgtctcttctattatggatgcaggccttgttttagtcttttctgagggtattaaatgtggagtgtga

SEQ ID NO: 13 - protein, 1527 aa, Homo sapiens
MSLSFCGNNISSYNINDGVLQNSCFVDALNLVPHVFLLFITFPILFIGWGSQSSKVQIHHNTWLHFPGHNLRWILTFALLFVHVCEIAEGIVSDSRRESRHLHLFMPAVMGFVATTTSIVYYHNIETSNFPKLLLALFLYWVMAFITKTIKLVKYCQSGLDISNLRFCITGMMVILNGLLMAVEINVIRVRRYVFFMNPQKVKPPEDLQDLGVRFLQPFVNLLSKATYWWMNTLIISAHKKPIDLKAIGKLPIAMRAVTNYVCLKDAYEEQKKKVADHPNRTPSIWLAMYRAFGRPILLSSTFRYLADLLGFAGPLCISGIVQRVNETQNGTNNTTGISETLSSKEFLENAYVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSIFMNAAIPIAAVLATFVTHAYASGNNLKPAEAFASLSLFHILVTPLSLLFTVVRFAVKAIISVQKLNEFLLSDEIGDDSWRTGESSLPFESCKKHTGVQPKTINRKQPGRYHLDSYEQSTRRLRPAETEDIAIKVTNGYFSWGSGLATLSNIDIRIPTGQLTMIVGQVGCGKSSLLLAILGEMQTLEGKVHWSNVNESEPSFEATRSRNRYSVAYAAQKPWLLNATVEENITFGSPFNKQRYKAVTDACSLQPDIDLLPFGDQTEIGERGINLSGGQRQRICVARALYQNTNIVFLDDPFSALDIHLSDHLMQEGILKFLQDDKRTLVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWRYLTSGGFFLLILMIFSKLLKHSVIVAIDYWLATWTSEYSINNTGKADQTYYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLVALLPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDAVVTEGGENFSVGQRQLFCLARAFVRKSSILIMDEATASIDMATENILQKVVMTAFADRTVVTMAHRVSSIMDAGLVLVFSEGIKCGV

SEQ ID NO: 14 - DNA, 4650 bp, Homo sapiens
atgagcctttcattttgtggtaacaacatttcttcatataatatcaacgatggtgtactacaaaattcctgctttgtggatgccctcaacctggtccctcatgtctttctgttgtttatcacttttccaatattgtttattgggtgggggagccaaagctcaaaagtacaaattcaccacaacacatggcttcattttccgggacataacctgagatggattcttacattcgctctcctgtttgtgcatgtctgtgaaatagcagaaggcattgtttcagactcgcggcgggaatcaaggcacctccacctctttatgccagccgtgatgggattcgttgccactacaacatcgatagtgtattatcataatatcgaaacatcaaattttcctaaattacttttagccctgttcctgtattgggtaatggcctttattacaaaaacaataaaattggttaagtactgtcagtctggcttggacatatcaaacctgcgtttctgcatcacaggcatgatggtcatcttgaatgggctcttgatggctgtggagatcaatgtcattcgagtcaggagatatgtatttttcatgaatcctcagaaagtaaagcctcctgaagacctccaggatctgggagtgagatttcttcaaccatttgtgaatttgctgtcaaaagcaacatactggtggatgaacacacttattatatctgctcacaaaaagcctattgatctgaaggcaattggaaaattgccaatagcaatgagagcagtaacaaattatgtttgcctgaaagatgcatatgaagaacaaaagaaaaaagttgcagatcatccaaatcggactccatctatatggcttgcaatgtacagagcttttgggcgaccaattctacttagtagcacattccgctatctggctgatttactgggttttgctggacctctttgtatttctggaatagttcagcgtgtgaatgaaacccagaatgggacaaataacacaactggaatttcagaaaccctctcatcaaaggaatttcttgaaaacgcttacgttctagcagttcttctcttcttggctcttattctgcaaaggacatttttgcaggcttcctactatgtaaccatagagactggcattaacctccgtggagctctgctggccatgatttataataaaatccttaggctctctacgtctaacttatccatgggggagatgactctggggcagatcaacaacttagtcgccattgaaactaatcaactcatgtggtttttgttcctgtgtcccaatctatgggctatgcctgttcagatcataatgggcgtgattctgctctataatttacttggatcaagtgcattggtcggtgcagctgtcattgtgctccttgcgccaattcagtactttattgctacaaagttggcagaggctcagaaaagtacacttgattattccactgagagactcaagaaaacaaatgaaatattgaaaggcatcaaacttctaaaattgtatgcctgggaacacattttctgcaaaagtgtggaggaaacaagaatgaaagaactatctagtctcaaaacctttgcactatatacatcactctccatcttcatgaatgcagcaattcccatagcagctgttcttgctacatttgtgacccatgcgtatgccagtggaaacaatctgaaacctgcagaggcctttgcttcactgtctctcttccatatcctggtcacaccactgtccctgctcttcacggtggtcagatttgcagtcaaagccatcataagtgttcaaaagctgaatgagtttctcttgagtgatgagattggtgacgacagttggcgaactggtgaaagttcgcttccttttgagtcctgtaagaagcacactggagttcagccaaaaactataaacaggaaacagcctggaagatatcacctggacagctatgagcaatcaacacggcgtctacgtcccgcagaaacagaggacattgcaataaaggtcacaaatggatacttttcatggggcagtggtttagctacattatccaatatagatattcgaattccaacaggtcagttaaccatgattgtgggccaagtaggatgtgggaagtcctctcttctccttgccatcctcggtgagatgcagacattggaaggaaaagttcactggagcaatgtaaatgaatctgagccttcttttgaagcaaccagaagtaggaacaggtactctgtggcatatgcagctcaaaagccttggctattaaatgctacagtagaagaaaatattacttttggaagtccttttaacaaacagaggtacaaagctgtcacagatgcctgttctcttcagccagatattgacttattaccatttggagatcaaactgaaattggagagaggggcatcaacctgagtgggggacagaggcagagaatctgtgtggcacgagcgctgtatcaaaacaccaacattgtctttttggatgatccattctcagccctggacattcacttgagtgatcatttaatgcaggaggggattttgaaattcctgcaagatgacaaaaggacactcgttcttgtgactcacaaattacagtatctgacgcatgctgactggatcatagccatgaaagatggaagtgtcctaagagaaggaactttgaaggacattcaaaccaaagatgttgagctttatgaacactggaaaacacttatgaatcggcaagatcaagaattagaaaaggatatggaagctgaccaaactactttagagaggaaaactctccgacgggccatgtattcaagagaagccaaagcccagatggaggacgaagacgaagaggaagaagaggaggaagatgaggatgataacatgtccactgtaatgaggctcaggactaaaatgccatggaaaacctgctggcgctacctgacatctggaggattcttcctgctcatcctgatgattttctctaagcttttgaagcattcggtcattgtagctatagactattggctggccacatggacatcggagtacagtataaacaatactggaaaagctgatcagacctactatgtggctggctttagcatactctgtggagcaggcattttcctttgccttgttacatccctcactgtagaatggatgggtctcacagctgccaaaaatcttcaccacaaccttctcaataagataatccttggaccaataaggttttttgataccacacccctgggactgattctcaatcgcttttcagctgatactaatatcattgatcagcacatccctccaaccttggaatctctaactcgctcaacactgctctgcctgtctgccattgggatgatttcttatgctactcctgtgttcctggttgctctcctgccccttggtgttgccttttattttatccagaaatactttcgggttgcctctaaggacctccaggaactcgacgatagtacccagctccctctgctctgtcacttctcagaaacagcagaaggactcaccaccattcgggcctttaggcatgaaaccagatttaaacaacgtatgctggaactgacggatacaaacaacattgcctacttatttctctcagctgccaacagatggctggaggtcaggacggattatctgggagcttgcattgtcctcactgcatctatagcatccattagtgggtcttccaattctggattggtaggcttgggtcttctgtatgcacttacgataaccaattatttgaattgggttgtgaggaacttggctgacctggaggtccagatgggtgcagtgaagaaggtgaacagtttcctgactatggagtcagagaactatgaaggcacaatggatccttctcaagttccagaacattggccacaagaaggggagatcaagatacatgatctgtgtgtcagatatgaaaataatctgaaacctgttcttaagcacgtcaaggcttacatcaaacctggacaaaaggtgggcatatgtggtcgcactggcagtgggaaatcatcgttatctctggctttcttcagaatggttgatatatttgatggaaaaattgtcattgatgggatagacatttccaaattaccactgcacacactacgttctagactttcaatcattctgcaggatccaatactattcagtggttccattagatttaatttagatccagagtgcaaatgcacagatgacagactctgggaagccttagaaattgctcagctgaagaatatggtcaaatctctacctggaggtctagatgcggttgtcactgaaggtggggagaattttagcgtgggacagagacagctattttgccttgccagggcctttgtccgcaaaagcagcattcttattatggatgaggcaacagcttccattgacatggccacagagaatattttgcaaaaagtagtaatgacagcctttgcagaccggaccgtggtgacaatggctcaccgtgtctcttctattatggatacaggccttgttttagtcttttctgagggtattttagtggagtgtgatactgtcccaaatttgttcgcccacaagaatggccccttttccactttggtgatgaccaacaagtag

SEQ ID NO: 15 - protein, 1549 aa, Homo sapiens
MSLSFCGNNISSYNINDGVLQNSCFVDALNLVPHVFLLFITFPILFIGWGSQSSKVQIHHNTWLHFPGHNLRWILTFALLFVHVCEIAEGIVSDSRRESRHLHLFMPAVMGFVATTTSIVYYHNIETSNFPKLLLALFLYWVMAFITKTIKLVKYCQSGLDISNLRFCITGMMVILNGLLMAVEINVIRVRRYVFFMNPQKVKPPEDLQDLGVRFLQPFVNLLSKATYWWMNTLIISAHKKPIDLKAIGKLPIAMRAVTNYVCLKDAYEEQKKKVADHPNRTPSIWLAMYRAFGRPILLSSTFRYLADLLGFAGPLCISGIVQRVNETQNGTNNTTGISETLSSKEFLENAYVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSIFMNAAIPIAAVLATFVTHAYASGNNLKPAEAFASLSLFHILVTPLSLLFTVVRFAVKAIISVQKLNEFLLSDEIGDDSWRTGESSLPFESCKKHTGVQPKTINRKQPGRYHLDSYEQSTRRLRPAETEDIAIKVTNGYFSWGSGLATLSNIDIRIPTGQLTMIVGQVGCGKSSLLLAILGEMQTLEGKVHWSNVNESEPSFEATRSRNRYSVAYAAQKPWLLNATVEENITFGSPFNKQRYKAVTDACSLQPDIDLLPFGDQTEIGERGINLSGGQRQRICVARALYQNTNIVFLDDPFSALDIHLSDHLMQEGILKFLQDDKRTLVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWRYLTSGGFFLLILMIFSKLLKHSVIVAIDYWLATWTSEYSINNTGKADQTYYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLVALLPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDAVVTEGGENFSVGQRQLFCLARAFVRKSSILIMDEATASIDMATENILQKVVMTAFADRTVVTMAHRVSSIMDTGLVLVFSEGILVECDTVPNLFAHKNGPFSTLVMTNK

SEQ ID NO: 16 - DNA, 4650 bp, Homo sapiens
atgagcctttcattttgtggtaacaacatttcttcatataatatcaacgatggtgtactacaaaattcctgctttgtggatgccctcaacctggtccctcatgtctttctgttgtttatcacttttccaatattgtttattgggtgggggagccaaagctcaaaagtacaaattcaccacaacacatggcttcattttccgggacataacctgagatggattcttacattcgctctcctgtttgtgcatgtctgtgaaatagcagaaggcattgtttcagactcgcggcgggaatcaaggcacctccacctctttatgccagccgtgatgggattcgttgccactacaacatcgatagtgtattatcataatatcgaaacatcaaattttcctaaattacttttagccctgttcctgtattgggtaatggcctttattacaaaaacaataaaattggttaagtactgtcagtctggcttggacatatcaaacctgcgtttctgcatcacaggcatgatggtcatcttgaatgggctcttgatggctgtggagatcaatgtcattcgagtcaggagatatgtatttttcatgaatcctcagaaagtaaagcctcctgaagacctccaggatctgggagtgagatttcttcaaccatttgtgaatttgctgtcaaaagcaacatactggtggatgaacacacttattatatctgctcacaaaaagcctattgatctgaaggcaattggaaaattgccaatagcaatgagagcagtaacaaattatgtttgcctgaaagatgcatatgaagaacaaaagaaaaaagttgcagatcatccaaatcggactccatctatatggcttgcaatgtacagagcttttgggcgaccaattctacttagtagcacattccgctatctggctgatttactgggttttgctggacctctttgtatttctggaatagttcagcgtgtgaatgaaacccagaatgggacaaataacacaactggaatttcagaaaccctctcatcaaaggaatttcttgaaaacgcttacgttctagcagttcttctcttcttggctcttattctgcaaaggacatttttgcaggcttcctactatgtaaccatagagactggcattaacctccgtggagctctgctggccatgatttataataaaatccttaggctctctacgtctaacttatccatgggggagatgactctggggcagatcaacaacttagtcgccattgaaactaatcaactcatgtggtttttgttcctgtgtcccaatctatgggctatgcctgttcagatcataatgggcgtgattctgctctataatttacttggatcaagtgcattggtcggtgcagctgtcattgtgctccttgcgccaattcagtactttattgctacaaagttggcagaggctcagaaaagtacacttgattattccactgagagactcaagaaaacaaatgaaatattgaaaggcatcaaacttctaaaattgtatgcctgggaacacattttctgcaaaagtgtggaggaaacaagaatgaaagaactatctagtctcaaaacctttgcactatatacatcactctccatcttcatgaatgcagcaattcccatagcagctgttcttgctacatttgtgacccatgcgtatgccagtggaaacaatctgaaacctgcagaggcctttgcttcactgtctctcttccatatcctggtcacaccactgtccctgctcttcacggtggtcagatttgcagtcaaagccatcataagtgttcaaaagctgaatgagtttctcttgagtgatgagattggtgacgacagttggcgaactggtgaaagttcgcttccttttgagtcctgtaagaagcacactggagttcagccaaaaactataaacaggaaacagcctggaagatatcacctggacagctatgagcaatcaacacggcgtctacgtcccgcagaaacagaggacattgcaataaaggtcacaaatggatacttttcatggggcagtggtttagctacattatccaatatagatattcgaattccaacaggtcagttaaccatgattgtgggccaagtaggatgtgggaagtcctctcttctccttgccatcctcggtgagatgcagacattggaaggaaaagttcactggagcaatgtaaatgaatctgagccttcttttgaagcaaccagaagtaggaacaggtactctgtggcatatgcagctcaaaagccttggctattaaatgctacagtagaagaaaatattacttttggaagtccttttaacaaacagaggtacaaagctgtcacagatgcctgttctcttcagccagatattgacttattaccatttggagatcaaactgaaattggagagaggggcatcaacctgagtgggggacagaggcagagaatctgtgtggcacgagcgctgtatcaaaacaccaacattgtctttttggatgatccattctcagccctggacattcacttgagtgatcatttaatgcaggaggggattttgaaattcctgcaagatgacaaaaggacactcgttcttgtgactcacaaattacagtatctgacgcatgctgactggatcatagccatgaaagatggaagtgtcctaagagaaggaactttgaaggacattcaaaccaaagatgttgagctttatgaacactggaaaacacttatgaatcggcaagatcaagaattagaaaaggatatggaagctgaccaaactactttagagaggaaaactctccgacgggccatgtattcaagagaagccaaagcccagatggaggacgaagacgaagaggaagaagaggaggaagatgaggatgataacatgtccactgtaatgaggctcaggactaaaatgccatggaaaacctgctggcgctacctgacatctggaggattcttcctgctcatcctgatgattttctctaagcttttgaagcattcggtcattgtagctatagactattggctggccacatggacatcggagtacagtataaacaatactggaaaagctgatcagacctactatgtggctggctttagcatactctgtggagcaggcattttcctttgccttgttacatccctcactgtagaatggatgggtctcacagctgccaaaaatcttcaccacaaccttctcaataagataatccttggaccaataaggttttttgataccacacccctgggactgattctcaatcgcttttcagctgatactaatatcattgatcagcacatccctccaaccttggaatctctaactcgctcaacactgctctgcctgtctgccattgggatgatttcttatgctactcctgtgttcctggttgctctcctgccccttggtgttgccttttattttatccagaaatactttcgggttgcctctaaggacctccaggaactcgacgatagtacccagctccctctgctctgtcacttctcagaaacagcagaaggactcaccaccattcgggcctttaggcatgaaaccagatttaaacaacgtatgctggaactgacggatacaaacaacattgcctacttatttctctcagctgccaacagatggctggaggtcaggacggattatctgggagcttgcattgtcctcactgcatctatagcatccattagtgggtcttccaattctggattggtaggcttgggtcttctgtatgcacttacgataaccaattatttgaattgggttgtgaggaacttggctgacctggaggtccagatgggtgcagtgaagaaggtgaacagtttcctgactatggagtcagagaactatgaaggcacaatggatccttctcaagttccagaacattggccacaagaaggggagatcaagatacatgatctgtgtgtcagatatgaaaataatctgaaacctgttcttaagcacgtcaaggcttacatcaaacctggacaaaaggtgggcatatgtggtcgcactggcagtgggaaatcatcgttatctctggctttcttcagaatggttgatatatttgatggaaaaattgtcattgatgggatagacatttccaaattaccactgcacacactacgttctagactttcaatcattctgcaggatccaatactattcagtggttccattagatttaatttagatccagagtgcaaatgcacagatgacagactctgggaagccttagaaattgctcagctgaagaatatggtcaaatctctacctggaggtctagatgcggttgtcactgaaggtggggagaattttagcgtgggacagagacagctattttgccttgccagggcctttgtccgcaaaagcagcattcttattatggatgaggcaacagcttccattgacatggccacagagaatattttgcaaaaagtagtaatgacagcctttgcagaccggaccgtggtgacaatggctcaccgtgtctcttctattatggatgcaggccttgttttagtcttttctgagggtattttagtggagtgtgatactgtcccaaatttgttcgcccacaagaatggccccttttccactttggtgatgatcaacaagtag

SEQ ID NO: 17 - protein, 1549 aa, Homo sapiens
MSLSFCGNNISSYNINDGVLQNSCFVDALNLVPHVFLLFITFPILFIGWGSQSSKVQIHHNTWLHFPGHNLRWILTFALLFVHVCEIAEGIVSDSRRESRHLHLFMPAVMGFVATTTSIVYYHNIETSNFPKLLLALFLYWVMAFITKTIKLVKYCQSGLDISNLRFCITGMMVILNGLLMAVEINVIRVRRYVFFMNPQKVKPPEDLQDLGVRFLQPFVNLLSKATYWWMNTLIISAHKKPIDLKAIGKLPIAMRAVTNYVCLKDAYEEQKKKVADHPNRTPSIWLAMYRAFGRPILLSSTFRYLADLLGFAGPLCISGIVQRVNETQNGTNNTTGISETLSSKEFLENAYVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSIFMNAAIPIAAVLATFVTHAYASGNNLKPAEAFASLSLFHILVTPLSLLFTVVRFAVKAIISVQKLNEFLLSDEIGDDSWRTGESSLPFESCKKHTGVQPKTINRKQPGRYHLDSYEQSTRRLRPAETEDIAIKVTNGYFSWGSGLATLSNIDIRIPTGQLTMIVGQVGCGKSSLLLAILGEMQTLEGKVHWSNVNESEPSFEATRSRNRYSVAYAAQKPWLLNATVEENITFGSPFNKQRYKAVTDACSLQPDIDLLPFGDQTEIGERGINLSGGQRQRICVARALYQNTNIVFLDDPFSALDIHLSDHLMQEGILKFLQDDKRTLVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWRYLTSGGFFLLILMIFSKLLKHSVIVAIDYWLATWTSEYSINNTGKADQTYYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLVALLPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDAVVTEGGENFSVGQRQLFCLARAFVRKSSILIMDEATASIDMATENILQKVVMTAFADRTVVTMAHRVSSIMDAGLVLVFSEGILVECDTVPNLFAHKNGPFSTLVMINK

SEQ ID NO: 18 - protein, 45 aa, Homo sapiens; identical to SEQ ID NO: 3

SEQ ID NO: 19 - protein, 45 aa, Homo sapiens
HRVSSIMDAGLVLVFSEGILVECDTVPNLLAHKNGLFSTLVMINK

SEQ ID NO: 20 - protein, 45 aa, Mus musculus; identical to SEQ ID NO: 7

SEQ ID NO: 21 - protein, 45 aa, Rattus norvegicus; identical to SEQ ID NO: 6

SEQ ID NO: 22 - protein, 45 aa, Oryctolagus cuniculus; identical to SEQ ID NO: 8

SEQ ID NO: 23 - protein, 45 aa, Cavia porcellus; identical to SEQ ID NO: 9

SEQ ID NO: 24 - protein, 45 aa, Homo sapiens
HRVSSIMDAGLVLVFSEGILVECDTVPNLFAHKNGPFSTLVMTNK

SEQ ID NO: 25 - protein, 45 aa, Homo sapiens
HRVHTILTADLVIVMKRGNILEYDTPESLLAQENGVFASFVRADM

SEQ ID NO: 26 - protein, 45 aa, Homo sapiens
HRVHTILSADLVIVLKRGAILEFDKPEKLLSRKDSVFASFVRADK